(19) United States
(12) Patent Application Publication
(10) Pub. No.: US 2026/0286407 A1
(43) Pub. Date: Sep. 24, 2026

(54) IN VIVO METHOD FOR THE PRODUCTION OF TULIPALIN A VIA ITACONIC ACID

(71) Applicants: BASE SE, Ludwigshafen am Rhein (DE); Max-Planck-Gesellschaft zur Férderung der Wissenschaften e.V., München (DE)

(72) Inventors: Srividhya SUNDARAM, Ludwigshafen am Rhein (DE); Barbara NAVÉ, Ludwigshafen am Rhein (DE); Tobias ERB, Marburg (DE); Joeline XIBERRAS, Ludwigshafen am Rhein (DE); Birgit HOFF, Ludwigshafen am Rhein (DE); Oskar ZELDER, Ludwigshafen am Rhein (DE); Patrick KAISER, Ludwigshafen am Rhein (DE); Ingeborg HEUSCHKEL, Ludwigshafen am Rhein (DE); Robin MACK, Ludwigshafen am Rhein (DE); Philipp WICHMANN, Ludwigshafen am Rhein (DE); Patrick GERLINGER, München (DE)

(21) Appl. No.: 19/472,190

(22) PCT Filed: Apr. 3, 2024
(86) PCT No.: PCT/EP2024/059075

§ 371 Date: Oct. 3, 2025

(30) Foreign Application Priority Data
Apr. 4, 2023 (EP) 23166573.8

(51) Int. Cl. C12P 17/04; C12N 9/00; C12N 9/02; C12N 9/04; C12N 9/10; C12N 9/88

(52) U.S. Cl. CPC C12P 17/04; C12N 9/0006; C12N 9/0008; C12N 9/1025; C12N 9/88; C12N 9/93; C12Y 101/01061; C12Y 102/01076; C12Y 401/01006; C12Y 602/01005

(57) ABSTRACT

The present invention is concerned with the in vivo production of tulipalin A (α-methylene-γ-butyrolactone). Provided are methods for the in vivo production of tulipalin A, recombinant cells or organisms for in vivo production of tulipalin A, enzymes needed for the in vivo production of tulipalin A, and nucleic acids for expression of those enzymes.

### FIELD OF THE INVENTION

The present invention relates to the field of biochemical synthesis. Provided are methods for the in vivo production of tulipalin A, recombinant cells or organisms for the in vivo production of tulipalin A, enzymes for the in vivo production of tulipalin A, and nucleic acids for expression of those enzymes.

### BACKGROUND OF THE INVENTION

Tulipalin A (α-methylene-γ-butyrolactone) is a naturally occurring vinyl monomer found in the tulip Tulipa gesneriana, as well as in the genera Tulipa, Erythronium, Gagea, Alstroemeria, Bomarea and Spiraea. Tulipalins function as defensive chemicals in plants and can elicit allergic reactions in humans.

Tulipalin's exo-methylene double bond allows for chain growth polymerization of the monomers to form the polymeric compound poly(tulipalin A). Tulipalin A polymerizes in a manner similar to methyl methacrylate (MMA), a polymer used in the production of polymethyl methacrylate acrylic plastics (PMMA), also known as acrylic glass, Perspex or Plexiglas, and methacrylate-butadiene-styrene (MBS). Hence, tulipalin A is considered a cyclic analog of methyl methacrylate and has the potential to replace oil-based MMA monomers as a sustainable alternative. As a naturally occurring vinyl, tulipalin A lends biocompatibility, biodegradability, eco-friendly, and renewable characteristics to the resulting polymers. Tulipalin A readily copolymerizes with copolymerizing agents such as styrene, methacrylate monomers, or acrylonitrile. In polymer producing industries, tulipalin is used in the production of materials such as thermoplastics, coatings and aliphatic polyesters, a technologically important class of biodegradable polymers. Compositions comprising tulipalin copolymers or copolyesters are used for example in cast glass and molding materials, automotive coats and finishes, thermoplastic resins and implantable medical devices.

In plants, tulipalins are derived from tuliposides, which are sugar esters composed of D-Glucose and 4′-hydroxy-2′-methylenebutanoyl and/or 3′,4′-dihydroxy-2′-methylenebutanoyl side chains. 6-Tuliposide A and B can spontaneously form their lactonized aglycons, tulipalin A and B. Tulipalin A and B show antimicrobial and insecticidal activity and serve as a chemical defense mechanism in plants. Tuliposides are stored in all parts of the plants, and only seem to be converted to tulipalins upon infection or wounding of the plant, when a tuliposide-converting enzyme (TCE) catalyzes the conversion of tuliposides to tulipalins. Therefore, tulipalin levels in plants are typically low or barely detectable, and extraction of tulipalin A is not an economically viable option of tulipalin A production.

Due to its potential as a sustainable alternative to methyl methacrylate, tulipalin A is an important industrial polymer. Hence, there is a need for an improved method for producing tulipalin A on an industrial scale, including materials needed for such a production process such as enzymes, recombinant cells or organisms, and nucleic acids for expression of enzymes used in production methods. The invention described herein provides methods for the in vivo production of tulipalin A, recombinant cells or organisms for the in vivo production of tulipalin A, enzymes used in these methods or by these cells or organisms and nucleic acids encoding these enzymes. The methods, cells or organisms, as well as enzymes and nucleic acids of the present invention have surprisingly found to allow for an improved, economic one-pot biosynthesis, which may rely in fermented raw materials.

### SUMMARY OF THE INVENTION

The invention relates to an in vivo method for producing tulipalin A from itaconic acid. The in vivo pathway to derive tulipalin A from itaconic acid involves three enzymatically catalyzed reaction steps, hence, the invention comprises a first, second and third enzyme. Optionally, a fourth enzyme can be used.

The first reaction involves the formation of the intermediate itaconyl-CoA from itaconic acid and a source of CoA. After synthesis of itaconyl-CoA from itaconic acid, the itaconyl-CoA is reacted further to form the intermediate itaconate semialdehyde. Once itaconate semialdehyde is present, it is reacted using a third enzyme catalyzing the formation of 2-methylene-4-ol-butyric acid, 2-Methylene-4-ol-butyric acid is able to form tulipalin A spontaneously via internal lactonization.

Hence, in a first aspect, the present invention relates to an in vivo method for producing tulipalin A (α-methylene-γ-butyrolactone) from itaconic acid, the method comprising culturing at least one recombinant cell or organism selected from bacteria and fungi in a fermentation medium, said recombinant cell or organism comprising
one or more recombinant nucleic acid molecules encoding for succinate-CoA ligase as a first enzyme;
one or more recombinant nucleic acid molecules encoding for succinyl-CoA reductase as a second enzyme; and
one or more recombinant nucleic acid molecules encoding for 3-sulfolactaldehyde reductase or one or more recombinant nucleic acid molecules encoding for succinate semialdehyde reductase as a third enzyme.

In various embodiments, the recombinant cell or organism is a natural producer of itaconic acid. Alternatively or additionally, in various embodiments, the recombinant cell or organism comprises one or more recombinant nucleic acid molecules encoding for aconitate decarboxylase cadA and/or one or more recombinant nucleic acid molecules encoding for citrate synthase gltA.

In various embodiments, the fermentation medium comprises CaCO₃, preferably at least 3 g/L CaCO₃, more preferably at least 5 g/L CaCO₃, even more preferably at least 10 g/L CaCO₃. Alternatively or additionally, in some embodiments, the fermentation medium comprises at least 0.1 g/L phosphate source, preferably at least 0.2 g/L phosphate source, more preferably at least 0.4 g/L phosphate source, wherein the phosphate source is preferably selected from KH₂PO₄ and K₂HPO₄, and mixtures thereof, preferably the phosphate source is an equivalent mixture thereof.

In some embodiments, the method of the present invention further comprises the step of isolating tulipalin A. In various embodiments, the method of the present invention comprises the step of extracting tulipalin A from the fermentation medium by means of at least one organic solvent. In some such embodiments, the extracting is continuous.

In some such embodiments, the organic solvent is not harmful to the recombinant cell or organism. Alternatively or additionally, the organic solvent is selected from the group consisting of 2-tert-butylphenol, ethyl acetate, hexyl acetate, butyl acetate, 4-methyl-2-pentanon, cyclohexanol, dodecane, isopropyl myristate, n-hexane, n-heptane, oleyl alcohol, bis(2-ethylhexyl) phthalate (DEHP), ethyl oleate, tridecan, n-octanol, 1-hexanol, methyl tert-butyl ether, and mixtures thereof, preferably is 2-tert-butylphenol, ethyl acetate, hexyl acetate, butyl acetate, 4-methyl-2-pentanon or cyclohexanol.

In various embodiments, the extracting is performed at a pH in the range of about 4 to about 9, preferably in the range of about 5 to about 8.

In a second aspect, the present invention relates to a recombinant cell or organism, said recombinant cell or organism being selected from bacteria and fungi and comprising
one or more recombinant nucleic acid molecules encoding for succinate-CoA ligase as a first enzyme;
one or more recombinant nucleic acid molecules encoding for succinyl-CoA reductase as a second enzyme; and
one or more recombinant nucleic acid molecules encoding for 3-sulfolactaldehyde reductase or one or more recombinant nucleic acid molecules encoding for succinate semialdehyde reductase as a third enzyme,
wherein the recombinant cell or organism
(a) is a natural producer of itaconic acid and/or
(b) comprises one or more recombinant nucleic acid molecules encoding aconitate decarboxylase, preferably aconitate decarboxylase cadA, and/or one or more recombinant nucleic acid molecules encoding for citrate synthase gltA, preferably citrate synthase gltA.

In various embodiments of the first aspect of the present invention, i.e., the in vivo method for producing tulipalin A (α-methylene-γ-butyrolactone) from itaconic acid of the present invention, and/or of the second aspect of the invention, the in vivo method for producing tulipalin A (α-methylene-γ-butyrolactone) from itaconic acid, i.e., the recombinant cell or organism, said recombinant cell or organism of the present invention, the recombinant cell or organism is a glucose-fermenting cell or organism. In various embodiments of the first aspect of the present invention and/or the second aspect of the present invention, the recombinant or cell organism is selected from the group consisting of Escherichia coli wild type, Gluconobacter oxydans, Streptomyces coelicolor, Streptococcus thermophiles, Pseudomonas putida, Bacillus licheniformis, Bacillus subtilis, Corynebacterium glutamicum, Pseudozyma tsukubaensis, Ustilago maydis, Aspergillus niger, Aspergillus terreus, Trichoderma reesei, Pichia pastoris, Saccharomyces cerevisiae, Saccharomyces pombe and Yarrowia (candida) lipolytica, preferably, the recombinant cell or organism is Escherichia coli wild type, Escherichia coli strain Ita23, Escherichia coli Ita36A, Pseudozyma tsukubaensis wild type (H488), Pseudozyma tsukubaensis strain HR12, or Pseudozyma tsukubaensis strain M15, most preferably Pseudozyma tsukubaensis strain HR12, Escherichia coli with a knock-out of glutamate-cysteine ligase (gshA), Escherichia coli Ita36A or Escherichia coli Ita36A AI.

In some embodiments of the first aspect of the present invention and/or the second aspect of the present invention, the recombinant cell or organism produces at least 14 μM tulipalin A in 40 h, preferably at least 25 UM tulipalin A in 50 h.

In some embodiments of the first aspect of the present invention and/or the second aspect of the present invention, the succinate-CoA ligase as a first enzyme is SucCD, and/or the succinyl-CoA reductase as a second enzyme is from Clostridium kluyveri, and/or the 3-sulfolactaldehyde reductase as a third enzyme is YihU or the succinate semialdehyde reductase as a third enzyme is AKR7A2. For instance, in some embodiments, the succinate-CoA ligase as a first enzyme is SucCD and the succinyl-CoA reductase as a second enzyme is from Clostridium kluyveri. In some other embodiments, the succinate-CoA ligase as a first enzyme is SucCD and the 3-sulfolactaldehyde reductase as a third enzyme is YihU. In some other embodiments, the succinate-CoA ligase as a first enzyme is SucCD, and the succinate semialdehyde reductase as a third enzyme is AKR7A2. In some other embodiments, the succinyl-CoA reductase as a second enzyme is from Clostridium kluyveri and the 3-sulfolactaldehyde reductase as a third enzyme is YihU. In some other embodiments, the succinyl-CoA reductase as a second enzyme is from Clostridium kluyveri, and the succinate semialdehyde reductase as a third enzyme is AKR7A2. In some other embodiments, the succinate-CoA ligase as a first enzyme is SucCD, the succinyl-CoA reductase as a second enzyme is from Clostridium kluyveri, and the 3-sulfolactaldehyde reductase as a third enzyme is YihU. In some embodiments, the succinate-CoA ligase as a first enzyme is SucCD, the succinyl-CoA reductase as a second enzyme is from Clostridium kluyveri, and the succinate semialdehyde reductase as a third enzyme is AKR7A2.

In some embodiments of the first aspect of the present invention and/or the second aspect of the present invention, the succinate-CoA ligase SucCD is from Escherichia coli. Alternatively or additionally, in some embodiments, the succinate-CoA ligase SucCD consists of two subunits SucC and SucD, wherein the SucC subunit comprises an amino acid sequence with at least 70% identity to an amino acid sequence according to SEQ ID NO: 2 and wherein the SucD subunit comprises an amino acid sequence with at least 70% identity to an amino acid sequence according to SEQ ID NO: 4.

In some embodiments of the first aspect of the present invention and/or the second aspect of the present invention, the succinyl-CoA reductase comprises an amino acid sequence with at least 70% identity to an amino acid sequence according to SEQ ID NO: 18.

In some embodiments of the first aspect of the present invention and/or the second aspect of the present invention, the third enzyme is a 3-sulfolactaldehyde reductase, preferably YihU from Escherichia coli strain K12. Alternatively or additionally, in some embodiments, 3-sulfolactaldehyde reductase YihU comprises an amino acid sequence with at least 70% identity to an amino acid sequence according to SEQ ID NO: 30.

In some embodiments of the first aspect of the present invention and/or the second aspect of the present invention, the third enzyme is a succinate semialdehyde reductase, preferably from Homo sapiens, preferably third enzyme is a succinate semialdehyde reductase AKR7A2. Alternatively or additionally, the succinate semialdehyde reductase comprises an amino acid sequence with at least 70% identity to an amino acid sequence according to SEQ ID NO: 40.

In some embodiments of the first aspect of the present invention and/or the second aspect of the present invention, aconitate decarboxylase cadA is from Aspergillus terreus. Alternatively or additionally, in some embodiments, aconitate decarboxylase cadA comprises an amino acid sequence with at least 70% identity to an amino acid sequence according to SEQ ID NO: 46.

In some embodiments of the first aspect of the present invention and/or the second aspect of the present invention, citrate synthase gltA is from Corynebacterium glutamicum. Alternatively or additionally, in some embodiments, citrate synthase gltA comprises an amino acid sequence with at least 70% identity to an amino acid sequence according to SEQ ID NO: 48.

Finally, in a third aspect, the present invention relates to the use of a recombinant cell or organism according to the present invention for the in vivo production of tulipalin A (α-methylene-γ-butyrolactone).

### BRIEF DESCRIPTION OF THE FIGURES

FIG. 1: Pathway of tulipalin A production indicating the reactions catalyzed by the first, second, third and fourth enzyme of the invention.

FIG. 2: Pathway of tulipalin A production indicating the reactions catalyzed by SucCD (succinate-CoA ligase) as the first enzyme; Scr (succinyl-CoA reductase) as the second enzyme; and YihU (aldehyde reductase) as the third enzyme.

FIG. 3: Time-course formation of 2-methylene-4-hydroxybutyrate and tulipalin A using purified enzymes (SucCD, Scr (succinyl-CoA reductase) and AKR7A2) in vitro. Work-up of the enzymatic reaction mix with formic acid results in the formation of more tulipalin A (tulipalin A formic acid). This suggests that the spontaneous lactonization of 2-methylene-4-hydroxybutyrate to tulipalin A is further enhanced by acid-catalyzed lactonization.

FIG. 4: Metabolic scheme of the in vivo production of tulipalin A in E. coli strain ita36A AI harboring the plasmids pCadCS (encoding CadA and GltA) and pPW136 (encoding SucCD, Scr (succinyl-CoA reductase), and YihU).

FIG. 5: Growth pattern during in vivo tulipalin A production. Growth behavior of 3 biological replicates each of E. coli strain ita36A AI harboring the plasmid pCadCS (itaconate production pathway; closed circles) and of E. coli strain ita36A AI harboring both the plasmid pCadCS (itaconate production pathway) and the plasmid pPW136 (tulipalin production pathway; closed squares).

FIG. 6: Itaconate production in vivo. Production of itaconate in E. coli strain ita36A AI harboring the plasmid pCadCS (itaconate production pathway) and in E. coli strain ita36A AI harboring both the plasmid pCadCS (itaconate production pathway) and the plasmid pPW136 (tulipalin production pathway). Supernatant samples were taken and analysed via LC-MS/MS at the given timepoints. Shown are 3 biological replicates each.

FIG. 7: Tulipalin production in vivo. Production of Tulipalin A in E. coli strain ita36A AI harboring the plasmid pCadCS (itaconate production pathway) and in E. coli strain ita36A AI harboring both the plasmid pCadCS (itaconate production pathway) and the plasmid pPW136 (tulipalin production pathway). Supernatant samples were taken and analysed via LC-MS/MS at the given timepoints. Shown are 3 biological replicates each.

FIG. 8: Screening of organic solvents for tulipalin A extraction ex situ, “Extract” being the organic phase containing the solvent, “Raffinate” being the aqueous phase remaining after extraction.

FIG. 9: Screening of organic solvents for tulipalin A extraction in situ.

FIG. 10: Optimisation of P. tsukubaensis fermentation medium by means of increased concentrations of CaCO₃ (FIG. 10a: 0 g/l CaCO₃, FIG. 10b: 3 g/l CaCO₃, FIG. 10c: 6 g/l CaCO₃, FIG. 10d: 10 g/l CaCO₃, FIG. 10e: 20 g/l CaCO₃, FIG. 10f: 33 g/l CaCO₃) in the fermentation medium.

FIG. 11: Tulipalin production in vivo. Production of Tulipalin A in E. coli strain ita36A AI harboring the plasmid pCadCS (itaconate production pathway) and either the plasmid pPW136 or pPW157 (both tulipalin production pathway). Extracts were taken and analysed via HPLC.

FIG. 12: Tulipalin production with lysates from E. coli BL21AI ΔgshA::Kan harboring a plasmid encoding SucCD, Scr and AKR7A2 as well as a cis-aconitate decarboxylase from Aspergillus terreus and a citrate synthase from Corynebacterium glutamicum in comparison to tulipalin production in E. coli BL21 (DE3) harboring a first plasmid encoding SucCD, a second plasmid encoding Scr and a third plasmid encoding AKR7A2 and expressing glutamate-cysteine ligase (gsh).

FIG. 13: Pathway to produce Tulipalin A via Tuliposide A using SucCD, Scr, YihU or AKR7A2 and UDP-glycosyltransferase.

FIG. 14: Spectrophotometric characterization of MBP-UDP-glycosyltransferase

FIG. 15: LC-MS verification of Tuliposide A formation by UDP-glycosyltransferase. EIC for Tuliposide m/z in negative mode is shown.

### DETAILED DESCRIPTION OF THE INVENTION

### General Definitions

Before the invention is described in detail with respect to some of its preferred embodiments, the following general definitions are provided.

The present invention as illustratively described in the following may suitably be practiced in the absence of any element or elements, limitation or limitations, not specifically disclosed herein.

The present invention will be described with respect to particular embodiments and with reference to certain figures, but the invention is not limited thereto, the scope of protection being defined by the appended claims.

Where the term “comprising” is used in the present description and claims, it does not exclude other elements. For the purposes of the present invention, the term “consisting of” is considered to be a preferred embodiment of the term “comprising of”. If hereinafter a group is defined to comprise at least a certain number of embodiments, this is also to be understood to disclose a group which preferably consists only of these embodiments.

For the purposes of the present invention, the term “obtained” is considered to be a preferred embodiment of the term “obtainable”. If hereinafter e.g. a compound is defined to be obtainable from a specific source, this is also to be understood to disclose a compound which is obtained from this source.

Where an indefinite or definite article is used when referring to a singular noun, e.g., “a”, “an” or “the”, this includes a plural of that noun unless something else is specifically stated. The terms “about” or “approximately” in the context of the present invention denote an interval of accuracy that the person skilled in the art will understand to still ensure the technical effect of the feature in question. The term typically indicates deviation from the indicated numerical value of ±10%, and preferably of ±5%.

Technical terms are used by their common sense. If a specific meaning is conveyed to certain terms, definitions of terms will be given in the following in the context of which the terms are used.

The term “expression” or “gene expression” as used herein refers to the process of synthesis of a gene product, preferably a functional RNA or protein. Gene expression generally comprises DNA transcription, optionally RNA processing and in the case of protein-expressing genes, RNA translation.

For the purposes of the invention, “recombinant” (or transgenic) with regard to a cell or an organism means that the cell or organism contains a heterologous polynucleotide which is introduced by man by gene technology and with regard to a polynucleotide includes all those constructions brought about by man by gene technology/recombinant DNA techniques in which either
(a) the sequence of the polynucleotide or a part thereof, or
(b) one or more genetic control sequences which are operably linked with the polynucleotide, including but not limited to, a promoter, or
(c) both a) and b)
are not located in their wildtype genetic environment or have been modified.

The term “heterologous” (or exogenous or foreign or recombinant or non-native) polypeptide is defined herein as a polypeptide that is not native to the host cell, a polypeptide native to the host cell in which structural modifications, e.g., deletions, substitutions, and/or insertions, have been made by recombinant DNA techniques to alter the native polypeptide, or a polypeptide native to the host cell whose expression is quantitatively altered or whose expression is directed from a genomic location different from the native host cell as a result of manipulation of the DNA of the host cell by recombinant DNA techniques, or whose expression is quantitatively altered as a result of manipulation of the regulatory elements of the polynucleotide by recombinant DNA techniques e.g., a stronger promoter; or a polynucleotide native to the host cell, but integrated not within its natural genetic environment as a result of genetic manipulation by recombinant DNA techniques.

The terms “nucleic acid” or “nucleic acid molecule” or “nucleic acid sequence” or “nucleotide sequence” are used interchangeably herein to refer to a biomolecule composed of nucleotides. The nucleic acid molecule can be comprised within an eukaryotic or prokaryotic organism, a eukaryotic or prokaryotic cell, a cell nucleus or a cell organelle, as part of a genome or as an individual molecule; or it can be comprised within a plasmid, a vector, an artificial chromosome; a nucleic acid can also exist outside of a cell, in vesicles, viruses or freely circulating, it can be isolated in a suitable composition, in a fixed or frozen tissue or cell culture, or dried. The nucleic acid can be synthesized or naturally occurring, i.e. isolated from nature.

The terms “sequence Identity”, “% sequence identity”, “% identity”, “% identical” or “sequence alignment” are used interchangeably herein and refer to the comparison of a first nucleic acid sequence to a second nucleic acid sequence, or a comparison of a first amino acid sequence to a second amino acid sequence and is calculated as a percentage based on the comparison. The result of this calculation can be described as “percent identical” or “percent ID.” A sequence identity may be determined by a program, which produces an alignment, and calculates identity counting both mismatches at a single position and gaps at a single position as non-identical positions in final sequence identity calculation. The sequence identity is determined over the entire length of the first and second nucleic acid sequence.

According to this invention, a pairwise global alignment is produced, meaning that two sequences are aligned over their complete length, which is usually produced by using a mathematical approach, called alignment algorithm.

According to the invention, the alignment is generated by using the algorithm of Needleman and Wunsch (J. Mol. Biol. (1979) 48, p. 443-453). Preferably, the program “NEEDLE” (The European Molecular Biology Open Software Suite (EMBOSS)) is used for the purposes of the current invention, with using the programs default parameter (polynucleotides: gap open=10.0, gap extend=0.5 and matrix=EDNAFULL; polypeptides: gap open=10.0, gap extend=0.5 and matrix=EBLOSUM62). After aligning two sequences, in a second step, an identity value is determined from the alignment produced. For this purpose, the %-identity is calculated by dividing the number of identical residues by the length of the alignment region which is showing the respective sequence of the present invention over its complete length multiplied with 100: %-identity=(identical residues/length of the alignment region which is showing the respective sequence of the present invention over its complete length)*100.

For calculating the percent identity of two nucleic acid sequences the same applies as for the calculation of percent identity of two amino acid sequences with some specifications. For nucleic acid sequences encoding for a protein the pairwise alignment shall be made over the complete length of the coding region of the sequence of this invention from start to stop codon excluding introns. Introns present in the other sequence, to which the sequence of this invention is compared, shall also be removed for the pairwise alignment. After aligning two sequences, in a second step, an identity value is determined from the alignment produced. Percent identity is calculated by %-identity=(identical residues/length of the alignment region which is showing the sequence of the invention from start to stop codon excluding introns over its complete length)*100.

Moreover, the preferred alignment program for nucleic acid sequences implementing the Needleman and Wunsch algorithm (J. Mol. Biol. (1979) 48, p. 443-453) is “NEEDLE” (The European Molecular Biology Open Software Suite (EMBOSS)) with the programs default parameters (gapopen=10.0, gapextend=0.5 and matrix=EDNAFULL).

The term “encoded protein” or “encoded amino acid” refers a protein that consists of a chain of amino acids, which results from a sequence that is encoded by a nucleic acid molecule comprising three-nucleotide codons.

As used herein, the term “cellulose” refers to a polysaccharide consisting of a linear chain of β(1→4)-linked D-glucose units. Cellulose is a structural component of the primary cell wall of plants and is also found in algae, oomycetes of bacteria. In one embodiment, the cellulose used in the method of the invention is derived from raw plant material. The term “raw plant material” refers to a plant material that is minimally processed or unprocessed, a grass, stalk, fruit, seed, leaf, wood, petal, fiber or any other plant part, often a feedstock or raw biomass, a plant-derived biomaterial or a plant which has undergone the transformation required to prepare it for further processing or for transport, e.g. milling, pressing, shaping, flaking.

The term “fermenting” or “fermentation” refers to a process which converts sugars, such as glucose, into cellular energy under anaerobic conditions, producing ATP, fermentation product and CO₂. A “fermentation product” is one of the products of the fermentation process including organic acids or alcohols.

The term “culturing”, “cultivation”, or culture” refers to the cultivation of cells in a suitable culture medium and under suitable conditions such as a suitable temperature and suitable pH.

The term “culture medium” refers to a water-based solution containing one or more chemical compounds that can support the growth of cells.

The terms “producing” and “synthesizing” as used herein may be used interchangeably and refer to the chemical synthesis of a molecule. Chemical synthesis of a molecule can comprise one or more chemical reactions that can be catalyzed by one or more enzymes. Chemical synthesis of a molecule can take place within a cell or organism or within a cell-free environment.

The term “catalyzing” or “catalyze” as used herein when referring to an enzymatic reaction means to cause or accelerate the initiation or the progression of a chemical reaction. Enzymes may use cellular or thermal energy and/or proton or electron donors and acceptors while catalyzing reactions. Catalyzing means reducing the activation energy needed to start a reaction by weakening the chemical bonds, usually by temporarily bonding with the reacting molecules.

The “UniProt numbers” or “UniProt” or “UniProt Accession numbers” provided herein refer to the unique identifiers given to individual genes and proteins by the UniProt Consortium, which are available from their database at www.uniprot.org and commonly used as references in the field. UniProtKB (UniProt Knowledgebase) is a freely accessible database of protein sequence and functional information. The UniProt database includes manually annotated and reviewed entries (provided by the Swiss-Prot database) and automatically annotated and not manually reviewed entries (provided by TrEMBL database), many of which are derived from genome sequencing projects. TrEMBL includes translated coding sequences from the EMBL-Bank/GenBank/DDBJ nucleotide sequence database, and others.

The “EC numbers” as provided herein refer to the Enzyme Commission number, a numerical classification scheme for enzymes based on the chemical reactions they catalyze, including a system of enzyme nomenclature. If different enzymes catalyze the same reaction, they receive the same EC number, for example homologous enzymes from different organisms or non-homologous isofunctional enzymes. A database of EC numbers can be accessed for example at https://iubmb.qmul.ac.uk/enzyme/provided by the Nomenclature Committee of the International Union of Biochemistry and Molecular Biology.

### Methods of the Invention

The method of tulipalin A (α-methylene-γ-butyrolactone) production of the present invention is an in vivo method. In the context of the present invention, the method is performed within a recombinant cell or organism, more particularly within the cytosol of a recombinant cell or organism.

Thus, in a first aspect, the present invention relates to an in vivo method for producing tulipalin A (α-methylene-γ-butyrolactone) from itaconic acid, the method comprising culturing at least one recombinant cell or organism selected from bacteria and fungi in a fermentation medium, said recombinant cell or organism comprising
one or more recombinant nucleic acid molecules encoding for succinate-CoA ligase as a first enzyme;
one or more recombinant nucleic acid molecules encoding for succinyl-CoA reductase as a second enzyme; and
one or more recombinant nucleic acid molecules encoding for 3-sulfolactaldehyde reductase or one or more recombinant nucleic acid molecules encoding for succinate semialdehyde reductase as a third enzyme.

The enzymes will be defined and described herein below in more detail.

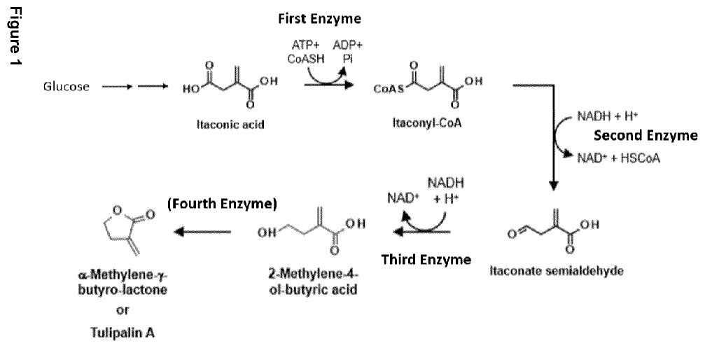

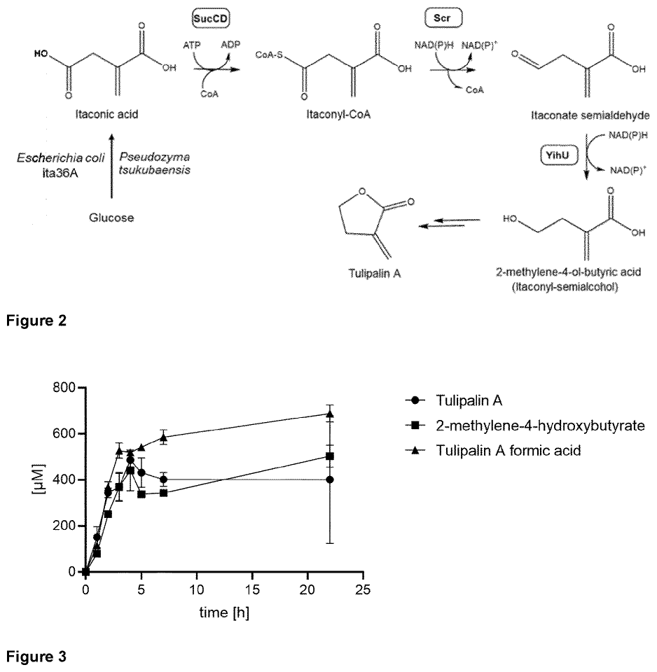

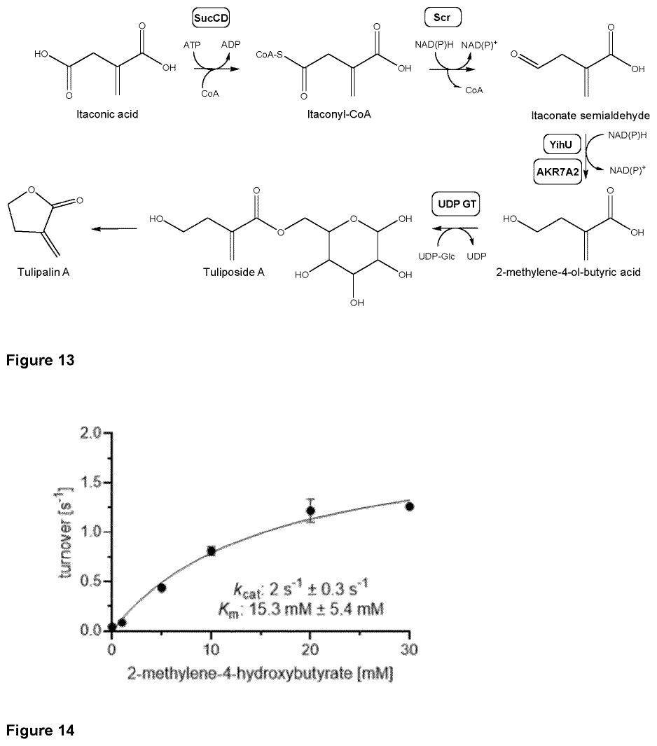

The first enzyme catalyzes the formation of itaconyl-CoA from itaconic acid, the second enzyme catalyzes the formation of itaconate semialdehyde from itaconyl-CoA, and the third enzyme catalyzes the formation of 2-methylene-4-ol-butyric acid from itaconate semialdehyde. In some embodiments, lactone formation of tulipalin A occurs spontaneously, i.e., without the catalyzing function of any enzyme. In optional embodiments, however, a fourth enzyme catalyzes lactone formation of 2-methylene-4-ol-butyric acid to produce tulipalin A. In an alternative optional embodiment, a fourth enzyme catalyzes the ester formation of 2-methylene-4-ol-butyric acid to produce 4-acetyloxy-2-methylene butanoic acid, which can be chemically converted to Tulipalin A. An overall reaction scheme in accordance with the methods of the present invention is detailed in FIG. 1. In an alternative optional embodiment, a fourth enzyme catalyzes the formation of Tuliposide A from 2-methylene-4-ol-butyric acid (see FIG. 13) and the Tuliposide A can be converted to Tulipalin A by a Tuliposide Converting Enzyme (TCE) or by basic hydrolysis followed by acidification to promote lactone formation.

In some embodiments, the starting compound for tulipalin A synthesis, itaconic acid, also called methylenesuccinic acid, is produced by fermentation. Starting material of fermentation may be raw plant material comprising cellulose, hemicellulose and/or starch. Raw materials may be cereal crops, grasses, grains, sugar beets, sugar cane, energy cane, sugar palm, potato, sweet potato or fruit. Cellulose, hemicellulose and starch are broken down into smaller carbohydrates including sucrose, glucose, lactose and fructose during liquefaction and saccharification of raw plant materials using amylolytic microorganisms or enzymes including α-amylases and glucoamylases. Glucose, or other starting materials such as sucrose, lactose, corn syrup, sugar beets, sugar cane, sugar palm or molasses, are then fermented by fermenting microorganisms that either naturally produce itaconic acid or have been engineered to produce itaconic acid from glucose. Methods for producing itaconic acid and organisms producing itaconic acid are known in the art (Regestein et al. Biotechnol. Biofuels 2018, Hossain et al. Fungal Biol. Biotechnol. 2019, Yang et al. JB&B 2019, Nemestothy et al. Waste Biomass Valorization 2020).

Instead of fermenting raw materials to produce glucose or other starting materials for itaconic acid production, the recombinant cell or organism in culture may be fed with glucose or molasses. In some embodiments, the recombinant cell or organism of the invention is cultured in a batch culture. Preferably, the recombinant cell or organism is cultured in a medium comprising glucose. In another embodiment, the recombinant cell or organism of the invention is cultured in a fed-batch culture. Preferably, the fed-batch culture is fed with a medium comprising glucose, glycerol or mixtures thereof.

In yet another embodiment, the recombinant cell or organism is cultured in a batch or fed-batch culture, preferably wherein the cultivation medium and the feeding medium comprise itaconic acid.

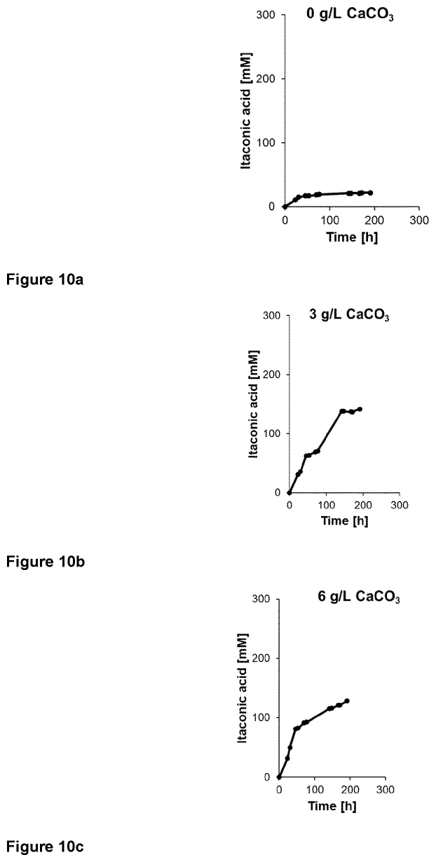

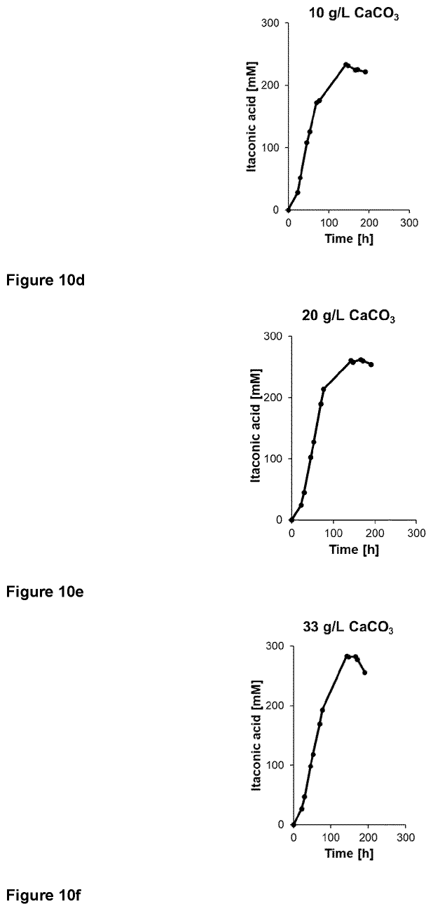

As surprisingly found by the present inventors, the production of itaconic acid and/or tulipalin A within the cell or organisms can be increased by increasing, when compared to conventional fermentation media, the amount of calcium and/or phosphate within the fermentation medium. In some such embodiments, the recombinant cell or organism is Pseudozyma tsukubaensis. Particularly, in various embodiments, the fermentation medium comprises CaCO₃, preferably at least 3 g/L CaCO₃, more preferably at least 5 g/L CaCO₃, even more preferably at least 10 g/L CaCO₃, for instance, but without limitation, 10, 11, 12, 13, 14, 15, 16, 17, 18, 19, 20, 21, 22, 23, 24, 25, 26, 27, 28, 29, 30, 31, 32, 33, 34, or 35 g/L CaCO₃ (FIG. 10). Alternatively or additionally, in some embodiments, the fermentation medium comprises at least 0.1 g/L phosphate source, preferably at least 0.2 g/L phosphate source, more preferably at least 0.4 g/L phosphate source, for instance, but without limitation, 0.4, 0.5, 0.6, 0.7, 0.8, 0.9, 1, 1.5, 2, 2.5, 3, 3.5, 4, 4.5, 5, 5.5, 6, 6.5, 7, 7.5, or 8 g/L phosphate source, preferably within the range of 0.4 to 2 g/L phosphate source, wherein the phosphate source is preferably selected from KH₂PO₄, K₂HPO₄, and mixtures thereof. Mixtures having a molar ratio of KH₂PO₄ and K₂HPO₄ in the range of 1:10 to 10:1, particularly in the range of 1:5 to 5:1, more particularly in the range if 1:3 to 3:1, such as in the range of 1:2 to 2:1, or 1:1, may preferably be used.

As further surprisingly found by the inventors, the production of itaconic acid and/or tulipalin A within the cell or organisms can be increased by using, as the nitrogen source, one or more of NaNO₃, NH₄Cl, and NH₄NO₃, preferably in a concentration in the range of 0.5 to 5 g/L, more preferably in the range of 1 to 3 g/L, for instance 1.5 to 2.5 g/L, for instance 2 g/L. In some preferred embodiments, the nitrogen source is NH₄NO₃, preferably in a concentration in the range of 0.5 to 5 g/L, more preferably in the range of 1 to 3 g/L, for instance 1.5 to 2.5 g/L, for instance 2 g/L. In some such embodiments, the recombinant cell or organism is Pseudozyma tsukubaensis.

Preferred fermentation media, particularly Pseudozyma tsukubaensis fermentation media, comprise, as the nitrogen source, NH₄NO₃, preferably in a concentration in the range of 0.5 to 5 g/L, more preferably in the range of 1 to 3 g/L, for instance 1.5 to 2.5 g/L, for instance 2 g/L; CaCO₃, preferably at least 3 g/L; CaCO₃, more preferably at least 5 g/L CaCO₃, even more preferably at least 10 g/L CaCO₃; and at least 0.1 g/L phosphate source, preferably at least 0.2 g/L phosphate source, more preferably at least 0.4 g/L phosphate source, wherein the phosphate source is preferably selected from KH₂PO₄, K₂HPO₄, and mixtures thereof.

In an alternative embodiment, the recombinant cell or organism may express a transporter protein facilitating uptake of itaconic acid into the cell. The recombinant cell or organism may either endogenously express the transporter protein or be transformed with a recombinant nucleic acid molecule encoding for the transporter protein. Recombinant cells or organisms endogenously expressing a transporter protein facilitating uptake of itaconic acid into the cell include, but are not limited to, Pseudomonas aeruginosa and Pseudomonas fluorescens. Alternatively, the recombinant cell or organism may be transformed with a nucleic acid molecule encoding for the transporter protein. Recombinant cells or organisms transformed with a nucleic acid molecule encoding for the transporter protein include, but are not limited to, E. coli or Pseudozyma tsukubaensis. In one embodiment, the transporter protein facilitating uptake of itaconic acid into the cell is an ABC transporter or a TRAP transporter. In one embodiment, the transporter protein facilitating uptake of itaconic acid into the cell is encoded by the nucleic acid sequences according to SEQ ID NO: 53, SEQ ID NO: 54 and SEQ ID NO: 55 and is composed of the proteins having the amino acid sequences according to SEQ ID NO: 56, SEQ ID NO: 57 and SEQ ID NO: 58. In one embodiment, the transporter protein facilitating uptake of itaconic acid into the cell has the amino acid sequence according to SEQ ID NO: 59.

After completion of the production process, the tulipalin A produced by the method of the present invention may be isolated by known methods. Therefore, in some embodiments, the method of the present invention further comprises the step of isolating tulipalin A. In some embodiments, the isolating of tulipalin A encompasses the isolating of tulipalin A from the fermentation medium, for instance by means of extraction. For instance, but without limitation, tulipalin A may be isolated by using organic solvents.

Therefore, in various embodiments, the method of the present invention further comprises the step of extracting tulipalin A from the fermentation medium by means of at least one organic solvent. Preferably, the extracting is continuous. The term “continuous”, in this context, means that the extracting of tulipalin A is performed in parallel to the culturing of the recombinant cell or organism, in other words, the culturing of the tulipalin A producing cell or organism is not terminated but continued at that point in time when tulipalin A is extracted from the fermentation medium, e.g., the extracting of tulipalin A neither requires nor results in the terminating of culturing of the recombinant cell or organisms and/or the in vivo producing of tulipalin A within the recombinant cell or organism.

In some such embodiments, the organic solvent used for extracting tulipalin A is not harmful to the recombinant cell or organism. In this context, the term “not harmful to the recombinant cell or organism” means that the growth of the recombinant cell or organism contacted with the respective organic solvent is decreased when compared to the growth of the recombinant cell or organism not contacted with the respective organic solvent by not more than 10%, preferably not more than 5%, more preferably not more than 1%; and/or that the glucose uptake of the recombinant cell or organism contacted with the respective organic solvent is decreased when compared to the glucose uptake of the recombinant cell or organism not contacted with the respective organic solvent by not more than 10%, preferably not more than 5%, more preferably not more than 1%; and/or that itaconic acid titres yielded with the recombinant cell or organism contacted with the respective organic solvent is decreased when compared to itaconic acid titres yielded with the recombinant cell or organism not contacted with the respective organic solvent by not more than 10%, preferably not more than 5%, more preferably not more than 1%.

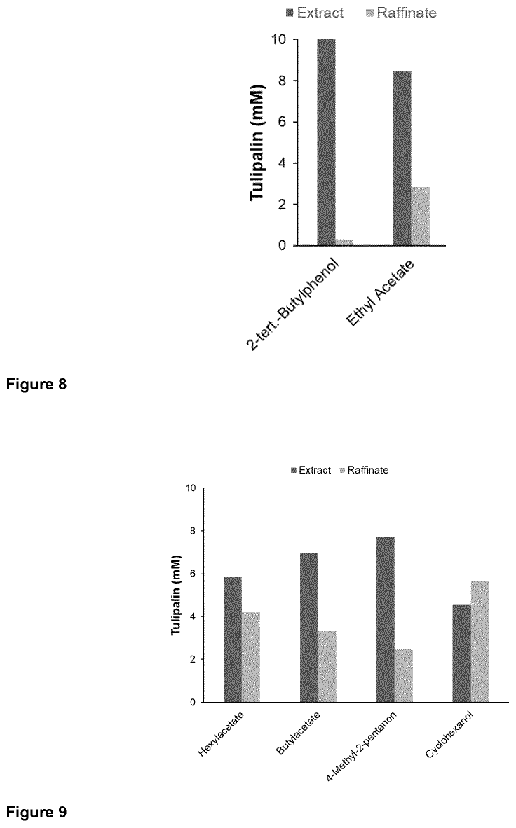

Non-limiting examples of organic solvents that may be used for the extracting of tulipalin A include 2-tert-butylphenol, ethyl acetate, dodecane, isopropyl myristate, n-hexane, n-heptane, oleyl alcohol, bis(2-ethylhexyl) phthalate (DEHP), ethyl oleate, 4-methyl-2-pentanon, butyl acetate, tridecan, n-octanol, cyclohexanol, 1-hexanol, methyl tert-butyl ether, hexyl acetate, and mixtures thereof, preferably 2-tert-butylphenol, ethyl acetate, cyclohexanol, and mixtures thereof (FIGS. 8 and 9).

In various embodiments, the extracting is continuous and, preferably, the solvent is selected from the group consisting of 4-methyl-2-pentanon, butyl acetate, tridecan, n-octanol, cyclohexanol, 1-hexanol, methyl tert-butyl ether, hexyl acetate, and mixtures thereof, preferably from the group consisting of hexylacetate, 4-methyl-2-pentanon, butyl acetate, cyclohexanol, and mixtures thereof, more preferably hexylacetate, 4-methyl-2-pentanon, butyl acetate, and mixtures thereof.

In various embodiments, the extracting is performed at a pH in the range of about 4 to about 9, for instance, but without limitation, at a pH of 4, 5, 6, 7, 8, or 9, preferably in the pH range of about 5 to about 8, for instance in a pH range of 6 to 7.

### Recombinant Cell or Organism

Recombinant cells or organisms useful in the method of the invention are cells or organisms that produce itaconic acid, either naturally or through genetic engineering. In one embodiment, the recombinant cell or organism produces itaconic acid, i.e., the recombinant cell or organism is a natural producer of itaconic acid. Cells or organisms that naturally produce itaconic acid include Aspergillus terreus, Aspergillus niger, Ustilago maydis and Pseudozyma tsukubaensis.

In another embodiment, the recombinant cell or organism is genetically engineered to produce itaconic acid. Such organisms include, for instance but without limitation, Escherichia coli strain Ita23, Escherichia coli Ita36A, Escherichia coli Ita36A AI and Pseudozyma tsukubaensis (see WO 2019/233853).

Other recombinant cells or organisms can be engineered to produce itaconic acid, including bacteria such as Escherichia coli, Gluconobacter oxydans, Streptomyces coelicolor, Streptococcus thermophiles, Pseudomonas putida, Bacillus licheniformis, Bacillus subtilis, Corynebacterium glutamicum, fungi or yeast such as Ustilago maydis, Aspergillus niger, Aspergillus terreus, Trichoderma reesei, Pichia pastoris, Saccharomyces cerevisiae, Saccharomyces pombe, Yarrowia (candida) lipolytica, or mammalian cell lines such as Chinese Hamster Ovary (CHO) cells, Hela cells or human embryonic kidney (HEK) 293 cells.

In some embodiments, the recombinant cell or organism is selected from the group consisting of Escherichia coli wild type, Gluconobacter oxydans, Streptomyces coelicolor, Streptococcus thermophiles, Pseudomonas fluorescens, Pseudomonas aeruginosa, Pseudomonas putida, Bacillus licheniformis, Bacillus subtilis, Corynebacterium glutamicum, Pseudozyma tsukubaensis, Ustilago maydis, Aspergillus niger, Aspergillus terreus, Trichoderma reesei, Pichia pastoris, Saccharomyces cerevisiae, Saccharomyces pombe, Ashbya gossypii and Yarrowia (candida) lipolytica.

In some embodiments, the Escherichia coli cell is selected from the strains wild type, MG1655, BI21, 60E4, Ita23 and Ita36A. In a preferred embodiment, the Escherichia coli cell is Escherichia coli Ita23 or Ita36A. In some preferred embodiments, the recombinant cell or organism is Escherichia coli wild type, Escherichia coli with a knock-out of glutamate-cysteine ligase (gshA), Escherichia coli strain Ita23, Escherichia coli Ita36A or Escherichia coli Ita36A AI.

In some embodiments, Escherichia coli Ita36A is further modified by integration of T7-RNA polymerase into the araB locus, placing the T7 RNA polymerase under the control of an arabinose inducible promoter. In such embodiments, in accordance with methods known in the art, the respective organism may be fed with glycerol instead of glucose so as to avoid catabolite repression.

In some embodiments, the Escherichia coli cell is further modified to knock-out the gene encoding for a glutamate-cysteine ligase (gsh) which abolishes glutathione biosynthesis. The inventors surprisingly found that the use of a glutathione-deficient E. coli cell increases Tulipalin A biosynthesis.

In some embodiments, the Escherichia coli cell is further modified to express a transporter facilitating uptake of itaconic acid into the cell, preferably the Escherichia coli cell is modified to express the transporter composed of the proteins having the amino acid sequences according to SEQ ID NO: 56, SEQ ID NO: 57 and SEQ ID NO: 58 or to express the transporter having the amino acid sequence according to SEQ ID NO: 59.

In some embodiments, the recombinant cell or organism is Pseudozyma tsukubaensis, preferably Pseudozyma tsukubaensis wild type (H488), Pseudozyma tsukubaensis strain HR12, or Pseudozyma tsukubaensis strain M15, most preferably Pseudozyma tsukubaensis strain HR12.

In some embodiments, the recombinant cell or organism is Pseudomonas sp., preferably the recombinant cell or organism is Pseudomonas fluorescens or Pseudomonas aeruginosa. These organisms naturally express a transporter for itaconic acid facilitating uptake of itaconic acid into the cell.

The present invention is also directed to a method for producing tulipalin A (α-methylene-γ-butyrolactone) from itaconic acid, the method comprising
(a) culturing at least one recombinant cell or organism selected from bacteria and fungi in a fermentation medium, said recombinant cell or organism comprising:
   one or more recombinant nucleic acid molecules encoding for succinate-CoA ligase as a first enzyme;
   one or more recombinant nucleic acid molecules encoding for succinyl-CoA reductase as a second enzyme; and
   one or more recombinant nucleic acid molecules encoding for 3-sulfolactaldehyde reductase or one or more recombinant nucleic acid molecules encoding for succinate semialdehyde reductase as a third enzyme;
(b) harvesting the recombinant cell or organism;
(c) subjecting the recombinant cell or organism to a treatment facilitating the uptake of itaconic acid by the recombinant cell or organism;
(d) incubating the recombinant cell or organism with an itaconic acid solution;
(e) isolating the tulipalin A.

In some embodiments, the treatment facilitating the uptake of itaconic acid comprises resuspending the harvested recombinant cell or organism in a buffer at low pH. In some embodiments, the pH of the buffer in which the harvested cells are resuspended is pH 4.0 to 5.0, preferably is pH 4.4.

In some embodiments, the treatment facilitating the uptake of itaconic acid comprises treating the recombinant cell or organism with a detergent which partially dissolves the membrane of the recombinant cell or organism. In some embodiments, the recombinant cell or organism is treated with Triton X-100, preferably with 1% Triton X-100.

In some embodiments, the treatment facilitating the uptake of itaconic acid comprises (a) freezing and thawing the recombinant cell or organism, (b) spray-drying the recombinant cell or organism and/or (c) lyophilizing the recombinant cell or organism.

In some embodiments, the recombinant cell or organism produces at least 14 UM tulipalin A in 40 h, preferably at least 25 UM tulipalin A in 50 h.

In some embodiments, the recombinant cell or organism is a glucose-fermenting cell or organism. In the context of the present invention, the term “glucose-fermenting cell or organism” is intended to refer to cells or organisms, which are capable of metabolize glucose as a carbon source. This does not preclude the capability of said cells or organisms to metabolize other carbon sources, such as glycerol.

In some embodiments, the recombinant cell or organism comprises one or more recombinant nucleic acid molecules encoding for aconitate decarboxylase cadA and/or one or more recombinant nucleic acid molecules encoding for citrate synthase gltA.

The term “aconitate decarboxylase” refers to a group of enzymes of class EC 4.1.1.6 that is able to catalyze the conversion of cis-aconitate to itaconate and CO₂.

The term “citrate synthase” refers to a group of enzymes of class E.C. 2.3.3.1 (previously 4.1.3.7) that is able to catalyze the aldol addition of acetyl-CoA and oxaloacetate, followed by hydrolysis, to citrate and CoA-SH.

In some embodiments, the aconitate decarboxylase cadA is from Aspergillus terreus (Uniprot ID B3IUN8). Alternatively or additionally, in some embodiments, the aconitate decarboxylase cadA comprises an amino acid sequence with at least 70% identity to an amino acid sequence according to SEQ ID NO: 46. In some embodiments, the aconitate decarboxylase cadA comprises an amino acid sequence with at least 75%, at least 80%, at least 85%, at least 90%, at least 95%, at least 98%, at least 99% or 100% identity to an amino acid sequence according to SEQ ID NO: 46.

In some embodiments, the citrate synthase gltA is from Corynebacterium glutamicum (Uniprot ID P42457). Alternatively or additionally, in some embodiments, the citrate synthase gltA comprises an amino acid sequence with at least 70% identity to an amino acid sequence according to SEQ ID NO: 48. In some embodiments, the citrate synthase gltA comprises an amino acid sequence with at least 75%, at least 80%, at least 85%, at least 90%, at least 95%, at least 98%, at least 99% or 100% identity to an amino acid sequence according to SEQ ID NO: 48.

In order to ensure tulipalin A formation and to stabilize intermediates of the tulipalin A synthesis pathway, it may be useful to reduce expression of aldose/aldehyde reductase (EC 1.1.1.21) in the recombinant cell or organism. In one embodiment, the recombinant cell or organism expresses reduced levels of endogenous aldehyde reductases compared to wild-type endogenous levels. Methods of engineering a cell or organism with reduced or abolished endogenous aldehyde reductase expression are known in the art (Kunjapur et al. J Am Chem Soc. 2014). In one embodiment, the recombinant cell or organism with reduced aldehyde reductase expression is Escherichia coli strain K12 MG1655.

In one aspect the present invention also provides for a recombinant cell or organism capable of producing tulipalin A. The invention provides recombinant cells or organisms capable of producing tulipalin A from itaconic acid. In other words, in the context of the present invention, the present invention provides for a recombinant cell or organism, said recombinant cell or organism being selected from bacteria and fungi and comprising one or more recombinant nucleic acid molecules encoding for succinate-CoA ligase as a first enzyme; one or more recombinant nucleic acid molecules encoding for succinyl-CoA reductase as a second enzyme; and one or more recombinant nucleic acid molecules encoding for 3-sulfolactaldehyde reductase or one or more recombinant nucleic acid molecules encoding for succinate semialdehyde reductase as a third enzyme, wherein the recombinant cell or organism is a natural producer of itaconic acid and/or comprises one or more recombinant nucleic acid molecules encoding for aconitate decarboxylase, preferably aconitate decarboxylase cadA, and/or one or more recombinant nucleic acid molecules encoding for citrate synthase gltA, preferably citrate synthase gltA. Therefore, in other words, the invention also relates to a recombinant cell or organism capable of carrying out the method of the invention.

In a further aspect, the present invention relates to the use of a recombinant cell or organism, as herein described and defined, for the in vivo production of tulipalin A (α-methylene-γ-butyrolactone).

In particular, the recombinant cell or organism comprises recombinant polypeptides for the expression of enzymes. These heterologous polypeptides comprise nucleic acid molecules encoding enzymes allowing for the production of tulipalin A from itaconic acid. These enzymes will be defined in the following in more detail.

Plasmids and respective expression cassettes can be designed and obtained using methods generally known in the art.

### Enzymes Used for the Production of Tulipalin A

The terms “first enzyme”, “second enzyme”, “third enzyme” and “fourth enzyme” as used herein refer to the order in which reaction steps of the production of tulipalin A from itaconic acid are described, as a matter of convenience. When expressing enzymes in a cell, the terms “first enzyme”, “second enzyme”, “third enzyme” and “fourth enzyme” do not refer to a specific order or sequence in which the enzymes are expressed. The enzymes may be expressed in any order. The enzymes may also be expressed in the order of first, second, third and fourth enzyme.

Itaconyl-CoA Synthesis from Itaconic Acid

The first enzyme of the invention catalyzes the formation of itaconyl-CoA from itaconic acid in the presence of a source of CoA. According to the invention, this reaction is catalyzed by a succinate-CoA ligase (EC 6.2.1.4 or EC 6.2.1.5).

The terms “Coenzyme A”, “CoA”, “SHCoA” or “CoASH” as used herein are used interchangeably and refer to the thiol Coenzyme A, a coenzyme used as a substrate by cellular enzymes, for example for oxidation of acids, such as during fatty acid synthesis or in the citric acid cycle. It occurs in both prokaryotic and eukaryotic genomes. CoA can react with carboxylic acids to form thioesters, thus functioning as an acyl group carrier. A molecule of Coenzyme A carrying an acyl group is referred to as “acyl-CoA”, for example Succinyl-CoA, Itaconyl-CoA, or Malonyl-CoA.

The terms “ligase” and “synthetase” are used interchangeably and refer to an enzyme that can catalyze the joining (“ligation”) of two molecules by forming a new chemical bond, typically via hydrolysis. In the context of the present invention, the terms “succinate-CoA ligase”, “succinyl coenzyme A synthetase”, “succinyl-CoA synthetase”, and “succinate thiokinase” are used interchangeably.

In one embodiment, the Succinyl-CoA synthetase is formed of two subunits beta and alpha. In one embodiment, the Succinyl-CoA synthetase is of bacterial origin, preferably Succinyl-CoA synthetase is isolated from a bacterium of the genus Escherichia, Advenella, Alcanivorax or Thermobifida. Succinyl-CoA synthetases are known to accept itaconic acid as a substrate (Schurmann et al. J Bacteriol. 2011).

In some embodiments, the Succinyl-CoA synthetase is from Escherichia coli (SucCD, subunit beta: SucC UniProt POA836 (SEQ ID NO: 2) and subunit alpha: SucD POAGE9 (SEQ ID NO: 4), Nolte et al. Appl Environ Microbiol. 2014), Advenella mimigardefordensis (SucCD, subunit beta: SucC Uniprot WOPFR9 (SEQ ID NO: 6) and subunit alpha: SucD Uniprot WOPAN5 (SEQ ID NO: 8)), Alcanivorax borkumensis (SucCD, subunit beta: SucC Uniprot QOVPF7 (SEQ ID NO: 10) and subunit alpha: SucD UniProt QOVPF8 (SEQ ID NO: 12), Schwander et al. Science 2016) or Thermobifida fusca (subunit beta: Tfu_2577 Uniprot Q47LR2 (SEQ ID NO: 14) and subunit alpha: Tfu_2576 UniProt Q47LR3 (SEQ ID NO: 16), Yang et al. Biotechnol Lett. 2020).

In some embodiments, the succinate-CoA ligase as the first enzyme is SucCD (ADP-forming, EC 6.2.1.5). In some embodiments, the succinate-CoA ligase SucCD is from Escherichia coli.

In one embodiment, subunit SucC of the Succinyl-CoA synthetase SucCD is at least 70% identical with an amino acid sequence according to SEQ ID NO: 2 and subunit SucD of the Succinyl-CoA synthetase (SucCD) is at least 70% identical with an amino acid sequence according to SEQ ID NO: 4. In some embodiments, subunit SucC of the Succinyl-CoA synthetase SucCD is at least 75%, at least 80%, at least 85%, at least 90%, at least 95%, at least 98%, at least 99% or 100% identical with an amino acid sequence according to SEQ ID NO: 2 and subunit SucD of the Succinyl-CoA synthetase SucCD is at least 75%, at least 80%, at least 85%, at least 90%, at least 95%, at least 98%, at least 99% or 100% identical with an amino acid sequence according to SEQ ID NO: 4.

Itaconate Semialdehyde Synthesis from Itaconyl-CoA

The second enzyme of the invention catalyzes the formation of Itaconate semialdehyde from Itaconyl-CoA in the presence of NAD (P) H/H⁺ as a cofactor. According to the invention, this reaction is catalyzed by a succinyl-CoA reductase (EC 1.2.1.76).

The term “semialdehyde” refers to the monoaldehyde of a dicarboxylic acid, i.e., wherein one of the two carboxylic acid functional groups forms an aldehyde functional group.

In some embodiments, the succinyl-CoA reductase is from Clostridium kluyveri (Scr, UniProt P38947 (SEQ ID NO: 18), Schürmann et al. J Bacteriol. 2011).

In some embodiments, the succinyl-CoA reductase is at least 70% identical with an amino acid sequence according to SEQ ID NO: 18. In some embodiments, Succinyl-CoA reductase is at least 75%, at least 80%, at least 85%, at least 90%, at least 95%, at least 98%, at least 99% or 100% identical with an amino acid sequence according to SEQ ID NO: 18.

2-Methylene-4-ol-butyric Acid Synthesis from Itaconate Semialdehyde

The third enzyme of the invention catalyzes the formation of 2-methylene-4-ol-butyric acid from Itaconate semialdehyde in the presence of NADH/H⁺ or NADPH/H⁺ According to the present invention, this reaction is catalyzed by a 3-sulfolactaldehyde reductase or, alternatively, by a succinate semialdehyde reductase.

The term “3-sulfolactaldehyde reductase” refers to a group of enzymes of class EC 1.1.1.373 that is able to catalyze the reduction of an aldehyde to a primary alcohol.

The term “succinate semialdehyde reductase” refers to a group of enzymes of class EC 1.1.1.11 that is able to catalyze the NADPH-dependent reduction of succinic semialdehyde to gamma-hydroxybutyrate.

In some embodiments, the third enzyme is a 3-sulfolactaldehyde reductase, wherein the 3-sulfolactaldehyde reductase comprises an amino acid sequence with at least 70% identity to an amino acid sequence according to SEQ ID NO: 30. In one embodiment, the 3-sulfolactaldehyde reductase comprises an amino acid sequence with at least 75%, at least 80%, at least 85%, at least 90%, at least 95%, at least 98%, at least 99% or 100% identity to an amino acid sequence according to SEQ ID NO: 30. In one embodiment, the 3-sulfolactaldehyde reductase is YihU from Escherichia coli, preferably from Escherichia coli strain K₁₂. In some embodiments, the 3-sulfolactaldehyde reductase is from Escherichia coli (YihU, UniProt POA9V8).

In some embodiments, the third enzyme is a succinate semialdehyde reductase, wherein the succinate semialdehyde reductase comprises an amino acid sequence with at least 70% identity to an amino acid sequence according to SEQ ID NO: 40. In one embodiment, the succinate semialdehyde reductase comprises an amino acid sequence with at least 75%, at least 80%, at least 85%, at least 90%, at least 95%, at least 98%, at least 99% or 100% identity to an amino acid sequence according to SEQ ID NO: 40. In one embodiment, the succinate semialdehyde reductase is AKR7A2 from Homo sapiens. In some embodiments, the succinate semialdehyde reductase is from Homo sapiens (AKR7A2, UniProt O43488, SEQ ID NO: 40).

Formation of Tulipalin A (α-methylene-γ-butyro-lactone)

The final step of tulipalin A synthesis is cyclic esterification of 2-methylene-4-ol-butyric acid to form tulipalin A (α-methylene-γ-butyro-lactone). Lactones are formed by intramolecular esterification of hydroxycarboxylic acids, which takes place spontaneously if the ring that is formed is five- or six-membered. Alternatively, the reaction mixture may be treated with acid to enhance lactone formation.

Formation of Tulipalin A from 2-Methylene-4-ol-butyric acid

In some embodiments of the present invention, the formation of tulipalin A from 2-methylene-4-ol-butyric acid occurs spontaneously through intramolecular esterification.

However, this step can also be catalyzed enzymatically. In one embodiment, the intramolecular esterification of 2-methylene-4-ol-butyric acid is catalyzed by an enzyme selected from mevalonolactone lactonase of Staphylococcus aureus (Drp35, UniProt Q99QV3, SEQ ID NO: 20, Reichert et al. Front Microbiol. 2018), 6-deoxyerythronolide synthase thioesterase from Saccharopolyspora erythraea (DEBS-TE, UniProt Q03133, SEQ ID NO: 22), lactimidomycin thioesterase from Streptomyces amphibiosporus (LtmG-TE, UniProt D8UYP5, SEQ ID NO: 24) and reveromycin thioesterase from Streptomyces sp. SN-593 (RevD-TE, UniProt G1UDV4, SEQ ID NO: 26).

In one embodiment, the intramolecular esterification of 2-methylene-4-ol-butyric acid involves the formation of a 2-methylene-4-ol-butyryl-CoA intermediate.

### Formation of Tulipalin A Via 4-Acetyloxy-2-Methylene Butanoic Acid

In an alternative embodiment, the fourth enzyme used in the invention catalyzes the formation of 4-acetyloxy-2-methylene butanoic acid from 2-methylene-4-ol-butyric acid. Enzymes useful for this purpose are acyl transferases (family VIII carboxyesterases) of the class EC 3.1.1. which catalyze the acyl transfer from acyl donors like ethyl- or vinyl-acetate to the primary OH of 2-methylene-4-ol-butyric acid or the alcohol acetyl-CoA transferases of the class EC 2.3.1.84 which transfer an acyl group from acetyl-CoA to the primary OH of 2-methylene-4-ol-butyric acid.

Hence, in some embodiments of the invention, the fourth enzyme is an acyltransferase selected from the group consisting of acyltransferase, carboxyesterase, carnitine acetyltransferase, galactoside O-acetyltransferase and alcohol acetyltransferase.

The term “acyl transferase” refers to a group of enzymes of class EC 3.1.1. that catalyze the acyl transfer between alcohols and acyl donors like ethyl acetate or vinyl acetate. Some carboxyesterases catalyze the reverse reaction of acyl transfer over hydrolysis.

In one embodiment, the acyl transfer to 2-methylene-4-ol-butyric acid is catalyzed by an enzyme selected from acyltransferase MsACT from Mycolicibacterium smegmatis (UniProt: AOR5U7, SEQ ID NO: 38), alcohol acetyl transferase ATF1 from Saccharomyces cerevisiae (UniProt: P40353, SEQ ID NO: 32), alcohol acetyl transferase ATF2 from Saccharomyces cerevisiae (UniProt: P53296, SEQ ID NO: 34), alcohol acetyl transferase Eat1 from Saccharomyces cerevisiae (UniProt: P53208, SEQ ID NO: 36), carnitine acetyltransferase YAT2 from Saccharomyces cerevisiae (UniProt: P40017, SEQ ID NO: 42), galactoside O-acetyltransferase LacA from Escherichia coli (UniProt: P07464, SEQ ID NO: 44), and acetyl COA geraniol/citronellol acetyltransferase from Rosa hybrid cultivar (UniProt: Q516B5, SEQ ID NO: 50).

4-Acetyloxy-2-methylene butanoic acid can further be chemically converted, more specifically acidified, to tulipalin A.

### Formation of Tulipalin A via Tuliposide A

In an alternative embodiment, the fourth enzyme used in the invention catalyzes the formation of Tuliposide A from 2-methylene-4-ol-butyric acid. Enzymes useful for this purpose are glycosyltransferases, preferably UDP-glycosyltransferases which catalyze the reaction between 2-methylene-4-ol-butyric acid and UDP-glucose. More preferably the UDP-glycosyltransferase has the amino acid sequence according to SEQ ID NO: 60.

In one embodiment the UDP-glycosyltransferase is fused to a tag which enhances solubility of the protein in a bacterial cell. Suitable tags enhancing solubility are described in Esposito and Chatterjee (2006) Curr. Opin. Biotechnol. 17 (4): 353-358 and include maltose binding protein (MBP), Strep-tag, SUMO-Tag, Trx-Tag, NusA-Tag, GST-Tag, SET-Tag, DsbC-Tag, Skp-Tag, T7PK-Tag, GB1-Tag and ZZ-Tag. Preferably, the tag enhancing solubility is MBP, more preferably the MBP has the amino acid sequence according to SEQ ID NO: 61 In some embodiments, the fusion protein of the UDP-glycosyltransferase and MBP has the sequence according to SEQ ID NO: 62.

After formation of Tuliposide A it can be converted to Tulipalin A using Tuliposide Converting Enzyme (TCE) which converts Tuliposide A into Tulipalin A and glucose. Suitable Tuliposide Converting Enzyme (TCE) are described in JP2010207211A, JP2012125162A, JP2014014277A, Nomura et al. (2019) Appl. Biochem. Biotechnol. 188:12-28, Kato et al. (2019) Bioorganic & Medicinal Chemistry Letters 29 (4): 664-667 and Kato et al. (2009) Bioscience, Biotechnology, and Biochemistry 73 (8): 1895-1897.

Alternatively, the Tuliposide A may be hydrolyzed by adding a base to promote hydrolysis of the glucose ester and subsequently acidified to promote lactonization of 2-methylene-4-hydroxybutyrate.

### NON-LIMITING EXEMPLARY EMBODIMENTS OF THE INVENTION

In various embodiments, provided is an in vivo method for producing tulipalin A (α-methylene-γ-butyrolactone) from itaconic acid, the method comprising culturing at least one recombinant cell or organism selected from bacteria and fungi in a fermentation medium, said recombinant cell or organism comprising
one or more recombinant nucleic acid molecules encoding for succinate-CoA ligase, preferably SucCD, as a first enzyme;
one or more recombinant nucleic acid molecules encoding for succinyl-CoA reductase, preferably from Clostridium kluyveri, as a second enzyme; and
one or more recombinant nucleic acid molecules encoding for 3-sulfolactaldehyde reductase, preferably YihU as a third enzyme,
   wherein the recombinant cell or organism is Escherichia coli Ita36A,
   wherein preferably the recombinant cell or organism comprises one or more recombinant nucleic acid molecules encoding aconitate decarboxylase, preferably aconitate decarboxylase cadA, and one or more recombinant nucleic acid molecules encoding for citrate synthase gltA, preferably citrate synthase gltA.

In some such embodiments, the succinate-CoA ligase SucCD is from Escherichia coli and consists of two subunits SucC and SucD, wherein preferably the SucC subunit comprises an amino acid sequence with at least 70% identity to an amino acid sequence according to SEQ ID NO: 2 and wherein the SucD subunit comprises an amino acid sequence with at least 70% identity to an amino acid sequence according to SEQ ID NO: 4, and the succinyl-CoA reductase is from Clostridium kluyveri and preferably comprises an amino acid sequence with at least 70% identity to an amino acid sequence according to SEQ ID NO: 18; and the 3-sulfolactaldehyde reductase YihU is from Escherichia coli strain K12, and preferably comprises an amino acid sequence with at least 70% identity to an amino acid sequence according to SEQ ID NO: 30, and in some preferred such embodiments the aconitate decarboxylase cadA is from Aspergillus terreus and preferably comprises an amino acid sequence with at least 70% identity to an amino acid sequence according to SEQ ID NO: 46; and the citrate synthase gltA is from Corynebacterium glutamicum and preferably comprises an amino acid sequence with at least 70% identity to an amino acid sequence according to SEQ ID NO: 48.

In some such embodiments, the method comprises the step of isolating tulipalin A, said step encompassing the extracting of tulipalin A from the fermentation medium by means of at least one organic solvent, wherein preferably the extracting is continuous.

For instance, in some embodiments, provided is an in vivo method for producing tulipalin A (α-methylene-γ-butyrolactone) from itaconic acid, the method comprising culturing at least one recombinant cell or organism selected from bacteria and fungi in a fermentation medium, said recombinant cell or organism comprising
one or more recombinant nucleic acid molecules encoding for succinate-CoA ligase, preferably SucCD, as a first enzyme;
one or more recombinant nucleic acid molecules encoding for succinyl-CoA reductase, preferably from Clostridium kluyveri, as a second enzyme; and
one or more recombinant nucleic acid molecules encoding for 3-sulfolactaldehyde reductase, preferably YihU as a third enzyme,
   wherein the recombinant cell or organism is Escherichia coli Ita36A,
   wherein the recombinant cell or organism comprises one or more recombinant nucleic acid molecules encoding aconitate decarboxylase, preferably aconitate decarboxylase cadA, and one or more recombinant nucleic acid molecules encoding for citrate synthase gltA, preferably citrate synthase gltA,
   wherein the succinate-CoA ligase SucCD is from Escherichia coli and consists of two subunits SucC and SucD, wherein the SucC subunit comprises an amino acid sequence with at least 70% identity to an amino acid sequence according to SEQ ID NO: 2 and wherein the SucD subunit comprises an amino acid sequence with at least 70% identity to an amino acid sequence according to SEQ ID NO: 4;
   wherein the succinyl-CoA reductase is from Clostridium kluyveri and comprises an amino acid sequence with at least 70% identity to an amino acid sequence according to SEQ ID NO: 18;
   wherein the 3-sulfolactaldehyde reductase YihU is from Escherichia coli strain K12 and comprises an amino acid sequence with at least 70% identity to an amino acid sequence according to SEQ ID NO: 30;
   wherein the aconitate decarboxylase cadA is from Aspergillus terreus and comprises an amino acid sequence with at least 70% identity to an amino acid sequence according to SEQ ID NO: 46; and
   wherein the citrate synthase gltA is from Corynebacterium glutamicum and comprises an amino acid sequence with at least 70% identity to an amino acid sequence according to SEQ ID NO: 48.

In some such embodiments, the method comprises the step of isolating tulipalin A, said step encompassing the extracting of tulipalin A from the fermentation medium by means of at least one organic solvent, wherein preferably the extracting is continuous.

For instance, in some embodiments, provided is an in vivo method for producing tulipalin A (α-methylene-γ-butyrolactone) from itaconic acid, the method comprising culturing at least one recombinant cell or organism selected from bacteria and fungi in a fermentation medium, said recombinant cell or organism comprising
one or more recombinant nucleic acid molecules encoding for succinate-CoA ligase, preferably SucCD, as a first enzyme;
one or more recombinant nucleic acid molecules encoding for succinyl-CoA reductase, preferably from Clostridium kluyveri, as a second enzyme; and
one or more recombinant nucleic acid molecules encoding for 3-sulfolactaldehyde reductase, preferably YihU as a third enzyme,
   wherein the recombinant cell or organism is Escherichia coli Ita36A,
   wherein the recombinant cell or organism comprises one or more recombinant nucleic acid molecules encoding aconitate decarboxylase, preferably aconitate decarboxylase cadA, and one or more recombinant nucleic acid molecules encoding for citrate synthase gltA, preferably citrate synthase gltA,
   wherein the succinate-CoA ligase SucCD is from Escherichia coli and consists of two subunits SucC and SucD, wherein the SucC subunit comprises an amino acid sequence with at least 70% identity to an amino acid sequence according to SEQ ID NO: 2 and wherein the SucD subunit comprises an amino acid sequence with at least 70% identity to an amino acid sequence according to SEQ ID NO: 4;
   wherein the succinyl-CoA reductase is from Clostridium kluyveri and comprises an amino acid sequence with at least 70% identity to an amino acid sequence according to SEQ ID NO: 18;
   wherein the 3-sulfolactaldehyde reductase YihU is from Escherichia coli strain K12 and comprises an amino acid sequence with at least 70% identity to an amino acid sequence according to SEQ ID NO: 30;
   wherein the aconitate decarboxylase cadA is from Aspergillus terreus and comprises an amino acid sequence with at least 70% identity to an amino acid sequence according to SEQ ID NO: 46; and
   wherein the citrate synthase gltA is from Corynebacterium glutamicum and comprises an amino acid sequence with at least 70% identity to an amino acid sequence according to SEQ ID NO: 48; and further
   wherein the method comprises the step of isolating tulipalin A, said step encompassing the extracting of tulipalin A from the fermentation medium by means of at least one organic solvent, wherein preferably the extracting is continuous.

In some such embodiments, the organic solvent is selected from the group consisting of 2-tert-butylphenol, ethyl acetate, hexyl acetate, butyl acetate, 4-methyl-2-pentanon, cyclohexanol, dodecane, isopropyl myristate, n-hexane, n-heptane, oleyl alcohol, bis(2-ethylhexyl) phthalate (DEHP), ethyl oleate, tridecan, n-octanol, 1-hexanol, methyl tert-butyl ether, and mixtures thereof, preferably is selected from the group consisting of hexylacetate, 4-methyl-2-pentanon, butyl acetate, cyclohexanol, and mixtures thereof, more preferably from the group consisting of hexylacetate, 4-methyl-2-pentanon, butyl acetate, and mixtures thereof.

For instance, in some embodiments, provided is an in vivo method for producing tulipalin A (α-methylene-γ-butyrolactone) from itaconic acid, the method comprising culturing at least one recombinant cell or organism selected from bacteria and fungi in a fermentation medium, said recombinant cell or organism comprising
one or more recombinant nucleic acid molecules encoding for succinate-CoA ligase, preferably SucCD, as a first enzyme;
one or more recombinant nucleic acid molecules encoding for succinyl-CoA reductase, preferably from Clostridium kluyveri, as a second enzyme; and
one or more recombinant nucleic acid molecules encoding for 3-sulfolactaldehyde reductase, preferably YihU as a third enzyme,
   wherein the recombinant cell or organism is Escherichia coli Ita36A,
   wherein the recombinant cell or organism comprises one or more recombinant nucleic acid molecules encoding aconitate decarboxylase, preferably aconitate decarboxylase cadA, and one or more recombinant nucleic acid molecules encoding for citrate synthase gltA, preferably citrate synthase gltA,
   wherein the succinate-CoA ligase SucCD is from Escherichia coli and consists of two subunits SucC and SucD, wherein the SucC subunit comprises an amino acid sequence with at least 70% identity to an amino acid sequence according to SEQ ID NO: 2 and wherein the SucD subunit comprises an amino acid sequence with at least 70% identity to an amino acid sequence according to SEQ ID NO: 4;
   wherein the succinyl-CoA reductase is from Clostridium kluyveri and comprises an amino acid sequence with at least 70% identity to an amino acid sequence according to SEQ ID NO: 18;
   wherein the 3-sulfolactaldehyde reductase YihU is from Escherichia coli strain K12 and comprises an amino acid sequence with at least 70% identity to an amino acid sequence according to SEQ ID NO: 30;
   wherein the aconitate decarboxylase cadA is from Aspergillus terreus and comprises an amino acid sequence with at least 70% identity to an amino acid sequence according to SEQ ID NO: 46; and
   wherein the citrate synthase gltA is from Corynebacterium glutamicum and comprises an amino acid sequence with at least 70% identity to an amino acid sequence according to SEQ ID NO: 48; and further
   wherein the method comprises the step of isolating tulipalin A, said step encompassing the extracting of tulipalin A from the fermentation medium by means of at least one organic solvent,
   wherein preferably the extracting is continuous;
   wherein the organic solvent is selected from the group consisting of 2-tert-butylphenol, ethyl acetate, hexyl acetate, butyl acetate, 4-methyl-2-pentanon, cyclohexanol, dodecane, isopropyl myristate, n-hexane, n-heptane, oleyl alcohol, bis(2-ethylhexyl) phthalate (DEHP), ethyl oleate, tridecan, n-octanol, 1-hexanol, methyl tert-butyl ether, and mixtures thereof, preferably is selected from the group consisting of hexylacetate, 4-methyl-2-pentanon, butyl acetate, cyclohexanol, and mixtures thereof, more preferably from the group consisting of hexylacetate, 4-methyl-2-pentanon, butyl acetate, and mixtures thereof.

In various other embodiments, provided is an in vivo method for producing tulipalin A (α-methylene-γ-butyrolactone) from itaconic acid, the method comprising culturing at least one recombinant cell or organism selected from bacteria and fungi in a fermentation medium, said recombinant cell or organism comprising
one or more recombinant nucleic acid molecules encoding for succinate-CoA ligase, preferably SucCD, as a first enzyme;
one or more recombinant nucleic acid molecules encoding for succinyl-CoA reductase, preferably from Clostridium kluyveri, as a second enzyme; and
one or more recombinant nucleic acid molecules encoding for succinate semialdehyde reductase, preferably AKR7A2, as a third enzyme,
   wherein the recombinant cell or organism is Escherichia coli Ita36A,
   wherein preferably the recombinant cell or organism comprises one or more recombinant nucleic acid molecules encoding aconitate decarboxylase, preferably aconitate decarboxylase cadA, and/or one or more recombinant nucleic acid molecules encoding for citrate synthase gltA, preferably citrate synthase gltA.

In some such embodiments, the succinate-CoA ligase SucCD is from Escherichia coli and consists of two subunits SucC and SucD, wherein preferably the SucC subunit comprises an amino acid sequence with at least 70% identity to an amino acid sequence according to SEQ ID NO: 2 and wherein the SucD subunit comprises an amino acid sequence with at least 70% identity to an amino acid sequence according to SEQ ID NO: 4; and the succinyl-CoA reductase is from Clostridium kluyveri and preferably comprises an amino acid sequence with at least 70% identity to an amino acid sequence according to SEQ ID NO: 18; and the 3-sulfolactaldehyde reductase semialdehyde reductase AKR7A2 is from Homo sapiens, and preferably comprises an amino acid sequence with at least 70% identity to an amino acid sequence according to SEQ ID NO: 40, and in some preferred such embodiments the aconitate decarboxylase cadA is from Aspergillus terreus and preferably comprises an amino acid sequence with at least 70% identity to an amino acid sequence according to SEQ ID NO: 46; and the citrate synthase gltA is from Corynebacterium glutamicum and preferably comprises an amino acid sequence with at least 70% identity to an amino acid sequence according to SEQ ID NO: 48.

For instance, in some embodiments, provided is an in vivo method for producing tulipalin A (α-methylene-γ-butyrolactone) from itaconic acid, the method comprising culturing at least one recombinant cell or organism selected from bacteria and fungi in a fermentation medium, said recombinant cell or organism comprising
one or more recombinant nucleic acid molecules encoding for succinate-CoA ligase, preferably SucCD, as a first enzyme;
one or more recombinant nucleic acid molecules encoding for succinyl-CoA reductase, preferably from Clostridium kluyveri, as a second enzyme; and
one or more recombinant nucleic acid molecules encoding for succinate semialdehyde reductase, preferably AKR7A2, as a third enzyme,
   wherein the recombinant cell or organism is Escherichia coli Ita36A,
   wherein the recombinant cell or organism comprises one or more recombinant nucleic acid molecules encoding aconitate decarboxylase, preferably aconitate decarboxylase cadA, and/or one or more recombinant nucleic acid molecules encoding for citrate synthase gltA, preferably citrate synthase gltA;
   wherein the succinate-CoA ligase SucCD is from Escherichia coli and consists of two subunits SucC and SucD, wherein the SucC subunit comprises an amino acid sequence with at least 70% identity to an amino acid sequence according to SEQ ID NO: 2 and wherein the SucD subunit comprises an amino acid sequence with at least 70% identity to an amino acid sequence according to SEQ ID NO: 4;
   wherein the succinyl-CoA reductase is from Clostridium kluyveri and comprises an amino acid sequence with at least 70% identity to an amino acid sequence according to SEQ ID NO: 18;
   wherein the 3-sulfolactaldehyde reductase semialdehyde reductase AKR7A2 is from Homo sapiens and comprises an amino acid sequence with at least 70% identity to an amino acid sequence according to SEQ ID NO: 40;
   wherein the aconitate decarboxylase cadA is from Aspergillus terreus and comprises an amino acid sequence with at least 70% identity to an amino acid sequence according to SEQ ID NO: 46; and
   wherein the citrate synthase gltA is from Corynebacterium glutamicum and comprises an amino acid sequence with at least 70% identity to an amino acid sequence according to SEQ ID NO: 48.

In some such embodiments, the method comprises the step of isolating tulipalin A, said step encompassing the extracting of tulipalin A from the fermentation medium by means of at least one organic solvent, wherein preferably the extracting is continuous.

For instance, in some embodiments, provided is an in vivo method for producing tulipalin A (α-methylene-γ-butyrolactone) from itaconic acid, the method comprising culturing at least one recombinant cell or organism selected from bacteria and fungi in a fermentation medium, said recombinant cell or organism comprising
one or more recombinant nucleic acid molecules encoding for succinate-CoA ligase, preferably SucCD, as a first enzyme;
one or more recombinant nucleic acid molecules encoding for succinyl-CoA reductase, preferably from Clostridium kluyveri, as a second enzyme; and
one or more recombinant nucleic acid molecules encoding for succinate semialdehyde reductase, preferably AKR7A2, as a third enzyme,
   wherein the recombinant cell or organism is Escherichia coli Ita36A,
   wherein the recombinant cell or organism comprises one or more recombinant nucleic acid molecules encoding aconitate decarboxylase, preferably aconitate decarboxylase cadA, and/or one or more recombinant nucleic acid molecules encoding for citrate synthase gltA, preferably citrate synthase gltA;
   wherein the succinate-CoA ligase SucCD is from Escherichia coli and consists of two subunits SucC and SucD, wherein the SucC subunit comprises an amino acid sequence with at least 70% identity to an amino acid sequence according to SEQ ID NO: 2 and wherein the SucD subunit comprises an amino acid sequence with at least 70% identity to an amino acid sequence according to SEQ ID NO: 4;
   wherein the succinyl-CoA reductase is from Clostridium kluyveri and comprises an amino acid sequence with at least 70% identity to an amino acid sequence according to SEQ ID NO: 18;
   wherein the 3-sulfolactaldehyde reductase semialdehyde reductase AKR7A2 is from Homo sapiens and comprises an amino acid sequence with at least 70% identity to an amino acid sequence according to SEQ ID NO: 40;
   wherein the aconitate decarboxylase cadA is from Aspergillus terreus and comprises an amino acid sequence with at least 70% identity to an amino acid sequence according to SEQ ID NO: 46; and
   wherein the citrate synthase gltA is from Corynebacterium glutamicum and comprises an amino acid sequence with at least 70% identity to an amino acid sequence according to SEQ ID NO: 48; and further
   wherein the method comprises the step of isolating tulipalin A, said step encompassing the extracting of tulipalin A from the fermentation medium by means of at least one organic solvent, wherein preferably the extracting is continuous.

In some such embodiments, the organic solvent is selected from the group consisting of 2-tert-butylphenol, ethyl acetate, hexyl acetate, butyl acetate, 4-methyl-2-pentanon, cyclohexanol, dodecane, isopropyl myristate, n-hexane, n-heptane, oleyl alcohol, bis(2-ethylhexyl) phthalate (DEHP), ethyl oleate, tridecan, n-octanol, 1-hexanol, methyl tert-butyl ether, and mixtures thereof, preferably is selected from the group consisting of hexylacetate, 4-methyl-2-pentanon, butyl acetate, cyclohexanol, and mixtures thereof, more preferably from the group consisting of hexylacetate, 4-methyl-2-pentanon, butyl acetate, and mixtures thereof.

For instance, in some embodiments, provided is an in vivo method for producing tulipalin A (α-methylene-γ-butyrolactone) from itaconic acid, the method comprising culturing at least one recombinant cell or organism selected from bacteria and fungi in a fermentation medium, said recombinant cell or organism comprising
one or more recombinant nucleic acid molecules encoding for succinate-CoA ligase, preferably SucCD, as a first enzyme;
one or more recombinant nucleic acid molecules encoding for succinyl-CoA reductase, preferably from Clostridium kluyveri, as a second enzyme; and
one or more recombinant nucleic acid molecules encoding for succinate semialdehyde reductase, preferably AKR7A2, as a third enzyme,
   wherein the recombinant cell or organism is Escherichia coli Ita36A,
   wherein the recombinant cell or organism comprises one or more recombinant nucleic acid molecules encoding aconitate decarboxylase, preferably aconitate decarboxylase cadA, and/or
   one or more recombinant nucleic acid molecules encoding for citrate synthase gltA, preferably citrate synthase gltA;
   wherein the succinate-CoA ligase SucCD is from Escherichia coli and consists of two subunits SucC and SucD, wherein the SucC subunit comprises an amino acid sequence with at least 70% identity to an amino acid sequence according to SEQ ID NO: 2 and wherein the SucD subunit comprises an amino acid sequence with at least 70% identity to an amino acid sequence according to SEQ ID NO: 4;
   wherein the succinyl-CoA reductase is from Clostridium kluyveri and comprises an amino acid sequence with at least 70% identity to an amino acid sequence according to SEQ ID NO: 18;
   wherein the 3-sulfolactaldehyde reductase semialdehyde reductase AKR7A2 is from Homo sapiens and comprises an amino acid sequence with at least 70% identity to an amino acid sequence according to SEQ ID NO: 40;
   wherein the aconitate decarboxylase cadA is from Aspergillus terreus and comprises an amino acid sequence with at least 70% identity to an amino acid sequence according to SEQ ID NO: 46; and
   wherein the citrate synthase gltA is from Corynebacterium glutamicum and comprises an amino acid sequence with at least 70% identity to an amino acid sequence according to SEQ ID NO: 48; and further
   wherein the method comprises the step of isolating tulipalin A, said step encompassing the extracting of tulipalin A from the fermentation medium by means of at least one organic solvent, wherein preferably the extracting is continuous;
   wherein the organic solvent is selected from the group consisting of 2-tert-butylphenol, ethyl acetate, hexyl acetate, butyl acetate, 4-methyl-2-pentanon, cyclohexanol, dodecane, isopropyl myristate, n-hexane, n-heptane, oleyl alcohol, bis(2-ethylhexyl) phthalate (DEHP), ethyl oleate, tridecan, n-octanol, 1-hexanol, methyl tert-butyl ether, and mixtures thereof, preferably is selected from the group consisting of hexylacetate, 4-methyl-2-pentanon, butyl acetate, cyclohexanol, and mixtures thereof, more preferably from the group consisting of hexylacetate, 4-methyl-2-pentanon, butyl acetate, and mixtures thereof.

In various other embodiments, provided is an in vivo method for producing tulipalin A (α-methylene-γ-butyrolactone) from itaconic acid, the method comprising culturing at least one recombinant cell or organism selected from bacteria and fungi in a fermentation medium, said recombinant cell or organism comprising
one or more recombinant nucleic acid molecules encoding for succinate-CoA ligase, preferably SucCD, as a first enzyme;
one or more recombinant nucleic acid molecules encoding for succinyl-CoA reductase, preferably from Clostridium kluyveri, as a second enzyme; and
one or more recombinant nucleic acid molecules encoding for 3-sulfolactaldehyde reductase, preferably YihU as a third enzyme,
   wherein the recombinant cell or organism is Pseudozyma tsukubaensis strain HR12,
   wherein preferably the recombinant cell or organism comprises one or more recombinant nucleic acid molecules encoding aconitate decarboxylase, preferably aconitate decarboxylase cadA, and one or more recombinant nucleic acid molecules encoding for citrate synthase gltA, preferably citrate synthase gltA.

In some such embodiments, the succinate-CoA ligase SucCD is from Escherichia coli and consists of two subunits SucC and SucD, wherein preferably the SucC subunit comprises an amino acid sequence with at least 70% identity to an amino acid sequence according to SEQ ID NO: 2 and wherein the SucD subunit comprises an amino acid sequence with at least 70% identity to an amino acid sequence according to SEQ ID NO: 4, and the succinyl-CoA reductase is from Clostridium kluyveri and preferably comprises an amino acid sequence with at least 70% identity to an amino acid sequence according to SEQ ID NO: 18; and the 3-sulfolactaldehyde reductase YihU is from Escherichia coli strain K₁₂, and preferably comprises an amino acid sequence with at least 70% identity to an amino acid sequence according to SEQ ID NO: 30, and in some preferred such embodiments the aconitate decarboxylase cadA is from Aspergillus terreus and preferably comprises an amino acid sequence with at least 70% identity to an amino acid sequence according to SEQ ID NO: 46; and the citrate synthase gltA is from Corynebacterium glutamicum and preferably comprises an amino acid sequence with at least 70% identity to an amino acid sequence according to SEQ ID NO: 48.

In some such embodiments, the method comprises the step of isolating tulipalin A, said step encompassing the extracting of tulipalin A from the fermentation medium by means of at least one organic solvent, wherein preferably the extracting is continuous.

For instance, in some embodiments, provided is an in vivo method for producing tulipalin A (α-methylene-γ-butyrolactone) from itaconic acid, the method comprising culturing at least one recombinant cell or organism selected from bacteria and fungi in a fermentation medium, said recombinant cell or organism comprising
one or more recombinant nucleic acid molecules encoding for succinate-CoA ligase, preferably SucCD, as a first enzyme;
one or more recombinant nucleic acid molecules encoding for succinyl-CoA reductase, preferably from Clostridium kluyveri, as a second enzyme; and
one or more recombinant nucleic acid molecules encoding for 3-sulfolactaldehyde reductase, preferably YihU as a third enzyme,
   wherein the recombinant cell or organism is Pseudozyma tsukubaensis strain HR12,
   wherein the recombinant cell or organism comprises one or more recombinant nucleic acid molecules encoding aconitate decarboxylase, preferably aconitate decarboxylase cadA, and one or more recombinant nucleic acid molecules encoding for citrate synthase gltA, preferably citrate synthase gltA,
   wherein the succinate-CoA ligase SucCD is from Escherichia coli and consists of two subunits SucC and SucD, wherein the SucC subunit comprises an amino acid sequence with at least 70% identity to an amino acid sequence according to SEQ ID NO: 2 and wherein the SucD subunit comprises an amino acid sequence with at least 70% identity to an amino acid sequence according to SEQ ID NO: 4;
   wherein the succinyl-CoA reductase is from Clostridium kluyveri and comprises an amino acid sequence with at least 70% identity to an amino acid sequence according to SEQ ID NO: 18;
   wherein the 3-sulfolactaldehyde reductase YihU is from Escherichia coli strain K12 and comprises an amino acid sequence with at least 70% identity to an amino acid sequence according to SEQ ID NO: 30;
   wherein the aconitate decarboxylase cadA is from Aspergillus terreus and comprises an amino acid sequence with at least 70% identity to an amino acid sequence according to SEQ ID NO: 46; and
   wherein the citrate synthase gltA is from Corynebacterium glutamicum and comprises an amino acid sequence with at least 70% identity to an amino acid sequence according to SEQ ID NO: 48.

In some such embodiments, the method comprises the step of isolating tulipalin A, said step encompassing the extracting of tulipalin A from the fermentation medium by means of at least one organic solvent, wherein preferably the extracting is continuous.

For instance, in some embodiments, provided is an in vivo method for producing tulipalin A (α-methylene-γ-butyrolactone) from itaconic acid, the method comprising culturing at least one recombinant cell or organism selected from bacteria and fungi in a fermentation medium, said recombinant cell or organism comprising
one or more recombinant nucleic acid molecules encoding for succinate-CoA ligase, preferably SucCD, as a first enzyme;
one or more recombinant nucleic acid molecules encoding for succinyl-CoA reductase, preferably from Clostridium kluyveri, as a second enzyme; and
one or more recombinant nucleic acid molecules encoding for 3-sulfolactaldehyde reductase, preferably YihU as a third enzyme,
   wherein the recombinant cell or organism is Pseudozyma tsukubaensis strain HR12,
   wherein the recombinant cell or organism comprises one or more recombinant nucleic acid molecules encoding aconitate decarboxylase, preferably aconitate decarboxylase cadA, and one or more recombinant nucleic acid molecules encoding for citrate synthase gltA, preferably citrate synthase gltA,
   wherein the succinate-CoA ligase SucCD is from Escherichia coli and consists of two subunits SucC and SucD, wherein the SucC subunit comprises an amino acid sequence with at least 70% identity to an amino acid sequence according to SEQ ID NO: 2 and wherein the SucD subunit comprises an amino acid sequence with at least 70% identity to an amino acid sequence according to SEQ ID NO: 4;
   wherein the succinyl-CoA reductase is from Clostridium kluyveri and comprises an amino acid sequence with at least 70% identity to an amino acid sequence according to SEQ ID NO: 18;
   wherein the 3-sulfolactaldehyde reductase YihU is from Escherichia coli strain K₁₂ and comprises an amino acid sequence with at least 70% identity to an amino acid sequence according to SEQ ID NO: 30;
   wherein the aconitate decarboxylase cadA is from Aspergillus terreus and comprises an amino acid sequence with at least 70% identity to an amino acid sequence according to SEQ ID NO: 46; and
   wherein the citrate synthase gltA is from Corynebacterium glutamicum and comprises an amino acid sequence with at least 70% identity to an amino acid sequence according to SEQ ID NO: 48; and further
   wherein the method comprises the step of isolating tulipalin A, said step encompassing the extracting of tulipalin A from the fermentation medium by means of at least one organic solvent,
   wherein preferably the extracting is continuous.

In some such embodiments, the organic solvent is selected from the group consisting of 2-tert-butylphenol, ethyl acetate, hexyl acetate, butyl acetate, 4-methyl-2-pentanon, cyclohexanol, dodecane, isopropyl myristate, n-hexane, n-heptane, oleyl alcohol, bis(2-ethylhexyl) phthalate (DEHP), ethyl oleate, tridecan, n-octanol, 1-hexanol, methyl tert-butyl ether, and mixtures thereof, preferably is selected from the group consisting of hexylacetate, 4-methyl-2-pentanon, butyl acetate, cyclohexanol, and mixtures thereof, more preferably from the group consisting of hexylacetate, 4-methyl-2-pentanon, butyl acetate, and mixtures thereof.

In some such embodiments, the fermentation medium comprises, as the nitrogen source, NH₄NO₃, preferably in a concentration in the range of 0.5 to 5 g/L, more preferably in the range of 1 to 3 g/L, for instance 1.5 to 2.5 g/L, for instance 2 g/L; and/or CaCO₃, preferably at least 3 g/L; CaCO₃, preferably at least 3 g/L CaCO₃, more preferably at least 5 g/L CaCO₃, even more preferably at least 10 g/L CaCO₃; and/or comprises at least 0.1 g/L phosphate source, preferably at least 0.2 g/L phosphate source, more preferably at least 0.4 g/L phosphate source, wherein the phosphate source is preferably selected from KH₂PO₄ and K₂HPO₄, and mixtures thereof, preferably the phosphate source is an equivalent mixture thereof.

For instance, in some embodiments, provided is an in vivo method for producing tulipalin A (α-methylene-γ-butyrolactone) from itaconic acid, the method comprising culturing at least one recombinant cell or organism selected from bacteria and fungi in a fermentation medium, said recombinant cell or organism comprising
one or more recombinant nucleic acid molecules encoding for succinate-CoA ligase, preferably SucCD, as a first enzyme;
one or more recombinant nucleic acid molecules encoding for succinyl-CoA reductase, preferably from Clostridium kluyveri, as a second enzyme; and
one or more recombinant nucleic acid molecules encoding for 3-sulfolactaldehyde reductase, preferably YihU as a third enzyme,
   wherein the recombinant cell or organism is Pseudozyma tsukubaensis strain HR12,
   wherein the recombinant cell or organism comprises one or more recombinant nucleic acid molecules encoding aconitate decarboxylase, preferably aconitate decarboxylase cadA, and one or more recombinant nucleic acid molecules encoding for citrate synthase gltA, preferably citrate synthase gltA,
   wherein the succinate-CoA ligase SucCD is from Escherichia coli and consists of two subunits SucC and SucD, wherein the SucC subunit comprises an amino acid sequence with at least 70% identity to an amino acid sequence according to SEQ ID NO: 2 and wherein the SucD subunit comprises an amino acid sequence with at least 70% identity to an amino acid sequence according to SEQ ID NO: 4;
   wherein the succinyl-CoA reductase is from Clostridium kluyveri and comprises an amino acid sequence with at least 70% identity to an amino acid sequence according to SEQ ID NO: 18;
   wherein the 3-sulfolactaldehyde reductase YihU is from Escherichia coli strain K₁₂ and comprises an amino acid sequence with at least 70% identity to an amino acid sequence according to SEQ ID NO: 30;
   wherein the aconitate decarboxylase cadA is from Aspergillus terreus and comprises an amino acid sequence with at least 70% identity to an amino acid sequence according to SEQ ID NO: 46; and
   wherein the citrate synthase gltA is from Corynebacterium glutamicum and comprises an amino acid sequence with at least 70% identity to an amino acid sequence according to SEQ ID NO: 48; and further
   wherein the method comprises the step of isolating tulipalin A, said step encompassing the extracting of tulipalin A from the fermentation medium by means of at least one organic solvent,
   wherein preferably the extracting is continuous;
   wherein the organic solvent is selected from the group consisting of 2-tert-butylphenol, ethyl acetate, hexyl acetate, butyl acetate, 4-methyl-2-pentanon, cyclohexanol, dodecane, isopropyl myristate, n-hexane, n-heptane, oleyl alcohol, bis(2-ethylhexyl) phthalate (DEHP), ethyl oleate, tridecan, n-octanol, 1-hexanol, methyl tert-butyl ether, and mixtures thereof, preferably is selected from the group consisting of hexylacetate, 4-methyl-2-pentanon, butyl acetate, cyclohexanol, and mixtures thereof, more preferably from the group consisting of hexylacetate, 4-methyl-2-pentanon, butyl acetate, and mixtures thereof.

In some such embodiments, the fermentation medium comprises, as the nitrogen source, NH₄NO₃, preferably in a concentration in the range of 0.5 to 5 g/L, more preferably in the range of 1 to 3 g/L, for instance 1.5 to 2.5 g/L, for instance 2 g/L; and/or CaCO₃, preferably at least 3 g/L; CaCO₃, preferably at least 3 g/L CaCO₃, more preferably at least 5 g/L CaCO₃, even more preferably at least 10 g/L CaCO₃, and/or comprises at least 0.1 g/L phosphate source, preferably at least 0.2 g/L phosphate source, more preferably at least 0.4 g/L phosphate source, wherein the phosphate source is preferably selected from KH₂PO₄ and K₂HPO₄, and mixtures thereof, preferably the phosphate source is an equivalent mixture thereof.

In various other embodiments, provided is an in vivo method for producing tulipalin A (α-methylene-γ-butyrolactone) from itaconic acid, the method comprising culturing at least one recombinant cell or organism selected from bacteria and fungi in a fermentation medium, said recombinant cell or organism comprising
one or more recombinant nucleic acid molecules encoding for succinate-CoA ligase, preferably SucCD, as a first enzyme;
one or more recombinant nucleic acid molecules encoding for succinyl-CoA reductase, preferably from Clostridium kluyveri, as a second enzyme; and
one or more recombinant nucleic acid molecules encoding for succinate semialdehyde reductase, preferably AKR7A2, as a third enzyme,
   wherein the recombinant cell or organism is Pseudozyma tsukubaensis strain HR12,
   wherein preferably the recombinant cell or organism comprises one or more recombinant nucleic acid molecules encoding aconitate decarboxylase, preferably aconitate decarboxylase cadA, and/or one or more recombinant nucleic acid molecules encoding for citrate synthase gltA, preferably citrate synthase gltA.

In some such embodiments, the succinate-CoA ligase SucCD is from Escherichia coli and consists of two subunits SucC and SucD, wherein preferably the SucC subunit comprises an amino acid sequence with at least 70% identity to an amino acid sequence according to SEQ ID NO: 2 and wherein the SucD subunit comprises an amino acid sequence with at least 70% identity to an amino acid sequence according to SEQ ID NO: 4; and the succinyl-CoA reductase is from Clostridium kluyveri and preferably comprises an amino acid sequence with at least 70% identity to an amino acid sequence according to SEQ ID NO: 18; and the 3-sulfolactaldehyde reductase semialdehyde reductase AKR7A2 is from Homo sapiens, and preferably comprises an amino acid sequence with at least 70% identity to an amino acid sequence according to SEQ ID NO: 40, and in some preferred such embodiments the aconitate decarboxylase cadA is from Aspergillus terreus and preferably comprises an amino acid sequence with at least 70% identity to an amino acid sequence according to SEQ ID NO: 46; and the citrate synthase gltA is from Corynebacterium glutamicum and preferably comprises an amino acid sequence with at least 70% identity to an amino acid sequence according to SEQ ID NO: 48.

For instance, in some embodiments, provided is an in vivo method for producing tulipalin A (α-methylene-γ-butyrolactone) from itaconic acid, the method comprising culturing at least one recombinant cell or organism selected from bacteria and fungi in a fermentation medium, said recombinant cell or organism comprising
one or more recombinant nucleic acid molecules encoding for succinate-CoA ligase, preferably SucCD, as a first enzyme;
one or more recombinant nucleic acid molecules encoding for succinyl-CoA reductase, preferably from Clostridium kluyveri, as a second enzyme; and
one or more recombinant nucleic acid molecules encoding for succinate semialdehyde reductase, preferably AKR7A2, as a third enzyme,
   wherein the recombinant cell or organism is Pseudozyma tsukubaensis strain HR12,
   wherein the recombinant cell or organism comprises one or more recombinant nucleic acid molecules encoding aconitate decarboxylase, preferably aconitate decarboxylase cadA, and/or
   one or more recombinant nucleic acid molecules encoding for citrate synthase gltA, preferably citrate synthase gltA;
   wherein the succinate-CoA ligase SucCD is from Escherichia coli and consists of two subunits SucC and SucD, wherein the SucC subunit comprises an amino acid sequence with at least 70% identity to an amino acid sequence according to SEQ ID NO: 2 and wherein the SucD subunit comprises an amino acid sequence with at least 70% identity to an amino acid sequence according to SEQ ID NO: 4;
   wherein the succinyl-CoA reductase is from Clostridium kluyveri and comprises an amino acid sequence with at least 70% identity to an amino acid sequence according to SEQ ID NO: 18;
   wherein the 3-sulfolactaldehyde reductase semialdehyde reductase AKR7A2 is from Homo sapiens and comprises an amino acid sequence with at least 70% identity to an amino acid sequence according to SEQ ID NO: 40;
   wherein the aconitate decarboxylase cadA is from Aspergillus terreus and comprises an amino acid sequence with at least 70% identity to an amino acid sequence according to SEQ ID NO: 46; and
   wherein the citrate synthase gltA is from Corynebacterium glutamicum and comprises an amino acid sequence with at least 70% identity to an amino acid sequence according to SEQ ID NO: 48.

In some such embodiments, the method comprises the step of isolating tulipalin A, said step encompassing the extracting of tulipalin A from the fermentation medium by means of at least one organic solvent, wherein preferably the extracting is continuous.

For instance, in some embodiments, provided is an in vivo method for producing tulipalin A (α-methylene-γ-butyrolactone) from itaconic acid, the method comprising culturing at least one recombinant cell or organism selected from bacteria and fungi in a fermentation medium, said recombinant cell or organism comprising
one or more recombinant nucleic acid molecules encoding for succinate-CoA ligase, preferably SucCD, as a first enzyme;
one or more recombinant nucleic acid molecules encoding for succinyl-CoA reductase, preferably from Clostridium kluyveri, as a second enzyme; and
one or more recombinant nucleic acid molecules encoding for succinate semialdehyde reductase, preferably AKR7A2, as a third enzyme,
   wherein the recombinant cell or organism is Pseudozyma tsukubaensis strain HR12,
   wherein the recombinant cell or organism comprises one or more recombinant nucleic acid molecules encoding aconitate decarboxylase, preferably aconitate decarboxylase cadA, and/or one or more recombinant nucleic acid molecules encoding for citrate synthase gltA, preferably citrate synthase gltA;
   wherein the succinate-CoA ligase SucCD is from Escherichia coli and consists of two subunits SucC and SucD, wherein the SucC subunit comprises an amino acid sequence with at least 70% identity to an amino acid sequence according to SEQ ID NO: 2 and wherein the SucD subunit comprises an amino acid sequence with at least 70% identity to an amino acid sequence according to SEQ ID NO: 4;
   wherein the succinyl-CoA reductase is from Clostridium kluyveri and comprises an amino acid sequence with at least 70% identity to an amino acid sequence according to SEQ ID NO: 18;
   wherein the 3-sulfolactaldehyde reductase semialdehyde reductase AKR7A2 is from Homo sapiens and comprises an amino acid sequence with at least 70% identity to an amino acid sequence according to SEQ ID NO: 40;
   wherein the aconitate decarboxylase cadA is from Aspergillus terreus and comprises an amino acid sequence with at least 70% identity to an amino acid sequence according to SEQ ID NO: 46; and
   wherein the citrate synthase gltA is from Corynebacterium glutamicum and comprises an amino acid sequence with at least 70% identity to an amino acid sequence according to SEQ ID NO: 48; and further
   wherein the method comprises the step of isolating tulipalin A, said step encompassing the extracting of tulipalin A from the fermentation medium by means of at least one organic solvent,
   wherein preferably the extracting is continuous.

In some such embodiments, the organic solvent is selected from the group consisting of 2-tert-butylphenol, ethyl acetate, hexyl acetate, butyl acetate, 4-methyl-2-pentanon, cyclohexanol, dodecane, isopropyl myristate, n-hexane, n-heptane, oleyl alcohol, bis(2-ethylhexyl) phthalate (DEHP), ethyl oleate, tridecan, n-octanol, 1-hexanol, methyl tert-butyl ether, and mixtures thereof, preferably is selected from the group consisting of hexylacetate, 4-methyl-2-pentanon, butyl acetate, cyclohexanol, and mixtures thereof, more preferably from the group consisting of hexylacetate, 4-methyl-2-pentanon, butyl acetate, and mixtures thereof.

In some such embodiments, the fermentation medium comprises, as the nitrogen source, NH₄NO₃, preferably in a concentration in the range of 0.5 to 5 g/L, more preferably in the range of 1 to 3 g/L, for instance 1.5 to 2.5 g/L, for instance 2 g/L; and/or CaCO₃, preferably at least 3 g/L; CaCO₃, preferably at least 3 g/L CaCO₃, more preferably at least 5 g/L CaCO₃, even more preferably at least 10 g/L CaCO₃; and/or comprises at least 0.1 g/L phosphate source, preferably at least 0.2 g/L phosphate source, more preferably at least 0.4 g/L phosphate source, wherein the phosphate source is preferably selected from KH₂PO₄ and K₂HPO₄, and mixtures thereof, preferably the phosphate source is an equivalent mixture thereof.

For instance, in some embodiments, provided is an in vivo method for producing tulipalin A (α-methylene-γ-butyrolactone) from itaconic acid, the method comprising culturing at least one recombinant cell or organism selected from bacteria and fungi in a fermentation medium, said recombinant cell or organism comprising
one or more recombinant nucleic acid molecules encoding for succinate-CoA ligase, preferably SucCD, as a first enzyme;
one or more recombinant nucleic acid molecules encoding for succinyl-CoA reductase, preferably from Clostridium kluyveri, as a second enzyme; and
one or more recombinant nucleic acid molecules encoding for succinate semialdehyde reductase, preferably AKR7A2, as a third enzyme,
   wherein the recombinant cell or organism is Pseudozyma tsukubaensis strain HR12,
   wherein the recombinant cell or organism comprises one or more recombinant nucleic acid molecules encoding aconitate decarboxylase, preferably aconitate decarboxylase cadA, and/or
   one or more recombinant nucleic acid molecules encoding for citrate synthase gltA, preferably citrate synthase gltA;
   wherein the succinate-CoA ligase SucCD is from Escherichia coli and consists of two subunits SucC and SucD, wherein the SucC subunit comprises an amino acid sequence with at least 70% identity to an amino acid sequence according to SEQ ID NO: 2 and wherein the SucD subunit comprises an amino acid sequence with at least 70% identity to an amino acid sequence according to SEQ ID NO: 4;
   wherein the succinyl-CoA reductase is from Clostridium kluyveri and comprises an amino acid sequence with at least 70% identity to an amino acid sequence according to SEQ ID NO: 18;
   wherein the 3-sulfolactaldehyde reductase semialdehyde reductase AKR7A2 is from Homo sapiens and comprises an amino acid sequence with at least 70% identity to an amino acid sequence according to SEQ ID NO: 40;
   wherein the aconitate decarboxylase cadA is from Aspergillus terreus and comprises an amino acid sequence with at least 70% identity to an amino acid sequence according to SEQ ID NO: 46; and
   wherein the citrate synthase gltA is from Corynebacterium glutamicum and comprises an amino acid sequence with at least 70% identity to an amino acid sequence according to SEQ ID NO: 48; and further
   wherein the method comprises the step of isolating tulipalin A, said step encompassing the extracting of tulipalin A from the fermentation medium by means of at least one organic solvent,
   wherein preferably the extracting is continuous;
   wherein the organic solvent is selected from the group consisting of 2-tert-butylphenol, ethyl acetate, hexyl acetate, butyl acetate, 4-methyl-2-pentanon, cyclohexanol, dodecane, isopropyl myristate, n-hexane, n-heptane, oleyl alcohol, bis(2-ethylhexyl) phthalate (DEHP), ethyl oleate, tridecan, n-octanol, 1-hexanol, methyl tert-butyl ether, and mixtures thereof, preferably is selected from the group consisting of hexylacetate, 4-methyl-2-pentanon, butyl acetate, cyclohexanol, and mixtures thereof, more preferably from the group consisting of hexylacetate, 4-methyl-2-pentanon, butyl acetate, and mixtures thereof.

In some such embodiments, the fermentation medium comprises, as the nitrogen source, NH₄NO₃, preferably in a concentration in the range of 0.5 to 5 g/L, more preferably in the range of 1 to 3 g/L, for instance 1.5 to 2.5 g/L, for instance 2 g/L; and/or CaCO₃, preferably at least 3 g/L; CaCO₃, preferably at least 3 g/L CaCO₃, more preferably at least 5 g/L CaCO₃, even more preferably at least 10 g/L CaCO₃; and/or comprises at least 0.1 g/L phosphate source, preferably at least 0.2 g/L phosphate source, more preferably at least 0.4 g/L phosphate source, wherein the phosphate source is preferably selected from KH₂PO₄ and K₂HPO₄, and mixtures thereof, preferably the phosphate source is an equivalent mixture thereof.

### The Invention is Also Described by the Following Items:

1. An in vivo method for producing tulipalin A (α-methylene-γ-butyrolactone) from itaconic acid, the method comprising culturing at least one recombinant cell or organism selected from bacteria and fungi in a fermentation medium, said recombinant cell or organism comprising
   one or more recombinant nucleic acid molecules encoding for succinate-CoA ligase as a first enzyme;
   one or more recombinant nucleic acid molecules encoding for succinyl-CoA reductase as a second enzyme; and
   one or more recombinant nucleic acid molecules encoding for 3-sulfolactaldehyde reductase or one or more recombinant nucleic acid molecules encoding for succinate semialdehyde reductase as a third enzyme.
2. Method according to item 1, wherein the recombinant cell or organism is a natural producer of itaconic acid.
3. Method according to item 1 or item 2, wherein the recombinant cell or organism comprises one or more recombinant nucleic acid molecules encoding for aconitate decarboxylase cadA and/or one or more recombinant nucleic acid molecules encoding for citrate synthase gltA.
4. Method according to item 3, wherein the recombinant cell or organism comprises one or more recombinant nucleic acid molecules encoding for aconitate decarboxylase cadA and one or more recombinant nucleic acid molecules encoding for citrate synthase gltA.
5. Method according to item 3 or item 4, wherein the aconitate decarboxylase cadA is from Aspergillus terreus.
6. Method according to any one of items 3-5, wherein the aconitate decarboxylase cadA comprises an amino acid sequence with at least 70% identity to an amino acid sequence according to SEQ ID NO: 46.
7. Method according to any one of items 3-6, wherein the aconitate decarboxylase cadA comprises an amino acid sequence with at least 75%, at least 80%, at least 85%, at least 90%, at least 95%, at least 98%, at least 99% or 100% identity to an amino acid sequence according to SEQ ID NO: 46.
8. Method according to any one of items 3-7, wherein the citrate synthase gltA is from Corynebacterium glutamicum.
9. Method according to any one of items 3-8, wherein the citrate synthase gltA comprises an amino acid sequence with at least 70% identity to an amino acid sequence according to SEQ ID NO: 48.
10. Method according to any one of items 3-9, wherein the citrate synthase gltA comprises an amino acid sequence with at least 75%, at least 80%, at least 85%, at least 90%, at least 95%, at least 98%, at least 99% or 100% identity to an amino acid sequence according to SEQ ID NO: 48.
11. Method according to any one of the preceding items, wherein the at least one recombinant cell or organism expresses a transporter protein facilitating uptake of itaconic acid into the cell.
12. Method according to item 11, wherein the transporter protein is an ABC transporter or a TRAP transporter.
13. Method according to item 11 or 12, wherein the transporter is composed of proteins having the amino acid sequence according to SEQ ID NO: 56, 57 and 58 or has the amino acid sequence according to SEQ ID NO: 59.
14. Method according to any one or items 1-13, wherein the recombinant cell or organism is a glucose-fermenting cell or organism.
15. Method according to any one of items 1-14, wherein the recombinant or cell organism is selected from the group consisting of Escherichia coli wild type, Gluconobacter oxydans, Streptomyces coelicolor, Streptococcus thermophiles, Pseudomonas fluorescens, Pseudomonas aeruginosa, Pseudomonas putida, Bacillus licheniformis, Bacillus subtilis, Corynebacterium glutamicum, Pseudozyma tsukubaensis, Ustilago maydis, Aspergillus niger, Aspergillus terreus, Trichoderma reesei, Pichia pastoris, Saccharomyces cerevisiae, Saccharomyces pombe and Yarrowia (candida) lipolytica, preferably, the recombinant cell or organism is Escherichia coli wild type, Escherichia coli strain Ita23, Escherichia coli Ita36A, Escherichia coli with a knock-out of glutamate-cysteine ligase (gshA), Pseudozyma tsukubaensis wild type (H488), Pseudozyma tsukubaensis strain HR12, or Pseudozyma tsukubaensis strain M15, most preferably Pseudozyma tsukubaensis strain HR12, Escherichia coli with a knock-out of glutamate-cysteine ligase (gshA), Escherichia coli Ita36A or Escherichia coli Ita36A AI.
16. Method according to any one of items 1-15, wherein the succinate-CoA ligase as a first enzyme is SucCD, and/or the succinyl-CoA reductase as a second enzyme is from Clostridium kluyveri, and/or the 3-sulfolactaldehyde reductase as a third enzyme is YihU or the succinate semialdehyde reductase as a third enzyme is AKR7A2.
17. Method according to any one of items 1-16, wherein the succinate-CoA ligase as a first enzyme is SucCD.
18. Method according to any one of items 1-16, wherein the succinyl-CoA reductase as a second enzyme is Scr.
19. Method according to any one of items 1-16, wherein the 3-sulfolactaldehyde reductase as a third enzyme is YihU.
20. Method according to any one of items 1-16, wherein the succinate semialdehyde reductase as a third enzyme is AKR7A2.
21. Method according to item 17, wherein the succinyl-CoA reductase as a second enzyme is Scr.
22. Method according to item 17 or item 21, wherein the 3-sulfolactaldehyde reductase as a third enzyme is YihU.
23. Method according to item 17 or item 21, wherein the succinate semialdehyde reductase as a third enzyme is AKR7A2.
24. Method according to any one of items 1-16, wherein the succinate-CoA ligase as the first enzyme is SucCD, and the succinyl-CoA reductase as the second enzyme is from Clostridium kluyveri, and the 3-sulfolactaldehyde reductase as the third enzyme is YihU.
25. Method according to any one of items 1-16, wherein the succinate-CoA ligase as the first enzyme is SucCD, and the succinyl-CoA reductase as the second enzyme is from Clostridium kluyveri, and the succinate semialdehyde reductase as the third enzyme is AKR7A2.
26. Method according to any one of items 1-25, wherein the Succinyl-CoA synthetase is from Escherichia coli (SucCD, subunit beta: SucC UniProt POA836 (SEQ ID NO: 2) and subunit alpha: SucD POAGE9 (SEQ ID NO: 4)), Advenella mimigardefordensis (SucCD, subunit beta: SucC Uniprot WOPFR9 (SEQ ID NO: 6) and subunit alpha: SucD Uniprot WOPAN5 (SEQ ID NO: 8)), Alcanivorax borkumensis (SucCD, subunit beta: SucC Uniprot QOVPF7 (SEQ ID NO: 10) and subunit alpha: SucD UniProt QOVPF8 (SEQ ID NO: 12)) or Thermobifida fusca (subunit beta: Tfu_2577 Uniprot Q47LR2 (SEQ ID NO: 14) and subunit alpha: Tfu_2576 UniProt Q47LR3 (SEQ ID NO: 16)).
27. Method according to any one of items 1-26, wherein the Succinyl-CoA synthetase is from Escherichia coli.
28. Method according to any one of items 16-27, wherein the succinate-CoA ligase SucCD consists of two subunits SucC and SucD, wherein the SucC subunit comprises an amino acid sequence with at least 70% identity to an amino acid sequence according to SEQ ID NO: 2 and wherein the SucD subunit comprises an amino acid sequence with at least 70% identity to an amino acid sequence according to SEQ ID NO: 4.
29. Method according to any one of items 16-28, wherein the succinate-CoA ligase SucCD consists of two subunits SucC and SucD, wherein subunit SucC of the Succinyl-CoA synthetase SucCD is at least 75%, at least 80%, at least 85%, at least 90%, at least 95%, at least 98%, at least 99% or 100% identical with an amino acid sequence according to SEQ ID NO: 2 and subunit SucD of the Succinyl-CoA synthetase SucCD is at least 75%, at least 80%, at least 85%, at least 90%, at least 95%, at least 98%, at least 99% or 100% identical with an amino acid sequence according to SEQ ID NO: 4.
30. Method according to any one of items 16 to 29, wherein the succinyl-CoA reductase is from Clostridium kluyveri.
31. Method according to any one of items 16 to 30, wherein the succinyl-CoA reductase comprises an amino acid sequence with at least 70% identity to an amino acid sequence according to SEQ ID NO: 18.
32. Method according to any one of items 16 to 31, wherein the succinyl-CoA reductase is at least 75%, at least 80%, at least 85%, at least 90%, at least 95%, at least 98%, at least 99% or 100% identical with an amino acid sequence according to SEQ ID NO: 18.
33. Method according to any one of items 1-22, 24, and 26-32, wherein the third enzyme is 3-sulfolactaldehyde reductase YihU from Escherichia coli strain K12.
34. Method according to any one of items 1-22, 24, and 26-33, wherein the third enzyme is 3-sulfolactaldehyde reductase YihU and comprises an amino acid sequence with at least 70% identity to an amino acid sequence according to SEQ ID NO: 30.
35. Method according to any one of items 1-22, 24, and 26-34, wherein the third enzyme is 3-sulfolactaldehyde reductase YihU and comprises an amino acid sequence with at least 75%, at least 80%, at least 85%, at least 90%, at least 95%, at least 98%, at least 99% or 100% identity to an amino acid sequence according to SEQ ID NO: 30.
36. Method according to any one of items 1-21, 23, and 25-32, wherein the third enzyme is succinate semialdehyde reductase and is from Homo sapiens.
37. Method according to any one of items 1-21, 23, 25-32, and 36, wherein the third enzyme is succinate semialdehyde reductase and comprises an amino acid sequence with at least 70% identity to an amino acid sequence according to SEQ ID NO: 40.
38. Method according to any one of items 1-21, 23, 25-32, and 36-37, wherein the third enzyme is succinate semialdehyde reductase and comprises an amino acid sequence with at least 75%, at least 80%, at least 85%, at least 90%, at least 95%, at least 98%, at least 99% or 100% identity to an amino acid sequence according to SEQ ID NO: 40.
39. Method according to any one of items 1-21, 23, 25-32, and 36-38, wherein the third enzyme is succinate semialdehyde reductase and is AKR7A2.
40. Method according to any one of items 1-22, 24, and 26-35, wherein
   the succinate-CoA ligase as the first enzyme is SucCD and consists of two subunits SucC and SucD, wherein the SucC subunit comprises an amino acid sequence with at least 70% identity to an amino acid sequence according to SEQ ID NO: 2 and wherein the SucD subunit comprises an amino acid sequence with at least 70% identity to an amino acid sequence according to SEQ ID NO: 4; and
   the succinyl-CoA reductase as the second enzyme is from Clostridium kluyveri and comprises an amino acid sequence with at least 70% identity to an amino acid sequence according to SEQ ID NO: 18, and
   the 3-sulfolactaldehyde reductase as the third enzyme is YihU and comprises an amino acid sequence with at least 70% identity to an amino acid sequence according to SEQ ID NO: 30.
41. Method according to any one of items 1-21, 23, 25-32, and 36-39, wherein
   the succinate-CoA ligase as the first enzyme is SucCD and consists of two subunits SucC and SucD, wherein the SucC subunit comprises an amino acid sequence with at least 70% identity to an amino acid sequence according to SEQ ID NO: 2 and wherein the SucD subunit comprises an amino acid sequence with at least 70% identity to an amino acid sequence according to SEQ ID NO: 4; and
   the succinyl-CoA reductase as the second enzyme is from Clostridium kluyveri and comprises an amino acid sequence with at least 70% identity to an amino acid sequence according to SEQ ID NO: 18, and
   the succinate semialdehyde reductase as the third enzyme comprises an amino acid sequence with at least 70% identity to an amino acid sequence according to SEQ ID NO: 40.
42. Method according to any one of items 1-41, wherein said recombinant cell or organism comprises one or more recombinant nucleic acid molecules encoding for an acyl transferase as a fourth enzyme.
43. Method according to item 42, wherein the acyl transferase has an amino acid sequence selected from the group consisting of SEQ ID NO: 32, SEQ ID NO: 34, SEQ ID NO: 36, SEQ ID NO: 38, SEQ ID NO: 42, SEQ ID NO: 44 and SEQ ID NO: 50.
44. Method according to any one of items 1-41, wherein said recombinant cell or organism comprises one or more recombinant nucleic acid molecules encoding for a glycosyltransferase as a fourth enzyme.
45. Method according to item 44, wherein the glycosyltransferase is a UDP-glycosyltransferase.
46. Method according to item 45, wherein the UDP-glycosyltransferase has the amino acid sequence according to SEQ ID NO: 60.
47. Method according to any one of items 44 to 46, wherein the glycosyltransferase comprises a tag which enhances solubility of the glycosyltransferase in the recombinant cell or organism.
48. Method according to item 47, wherein the tag which enhances solubility of the glycosyltransferase in the recombinant cell or organism is selected from the group consisting of: Maltose binding protein, Strep-Tag, SUMO-Tag, Trx-Tag, NusA-Tag, GST-Tag, SET-Tag, DsbC-Tag, Skp-Tag, T7PK-Tag, GB1-Tag and ZZ-Tag.
49. Method according to any one of items 1-48, wherein the fermentation medium comprises CaCO₃, preferably at least 3 g/L CaCO₃, more preferably at least 5 g/L CaCO₃, even more preferably at least 10 g/L CaCO₃.
50. Method according to any one of items 1-49, wherein the fermentation medium comprises at least 0.1 g/L phosphate source, preferably at least 0.2 g/L phosphate source, more preferably at least 0.4 g/L phosphate source, wherein the phosphate source is preferably selected from KH₂PO₄ and K₂HPO₄, and mixtures thereof, preferably an equivalent mixture thereof.
51. Method according to any one of items 1-50, wherein the fermentation medium comprises NH₄NO₃ as the nitrogen source.
52. Method according to any one of items 1-51, wherein the method further comprises the step of isolating tulipalin A.
53. Method according to item 52, wherein isolating of tulipalin A encompasses the step of extracting tulipalin A from the fermentation medium by means of at least one organic solvent.
54. Method according to item 53, wherein the extracting is continuous.
55. Method according to any one of items 53-54, wherein the organic solvent is not harmful to the recombinant cell or organism.
56. Method according to any one of items 53-55, wherein the organic solvent is selected from the group consisting of 2-tert-butylphenol, ethyl acetate, hexyl acetate, butyl acetate, 4-methyl-2-pentanon, cyclohexanol, dodecane, isopropyl myristate, n-hexane, n-heptane, oleyl alcohol, bis(2-ethylhexyl) phthalate (DEHP), ethyl oleate, tridecan, n-octanol, 1-hexanol, methyl tert-butyl ether, and mixtures thereof, preferably is 2-tert-butylphenol, ethyl acetate, hexyl acetate, butyl acetate, 4-methyl-2-pentanon or cyclohexanol.
57. Method according to any one of items 53-56, wherein the extracting is performed at a pH in the range of 4 to 9, preferably in the range of 5 to 8.
58. A recombinant cell or organism, said recombinant cell or organism being selected from bacteria and fungi and comprising
   one or more recombinant nucleic acid molecules encoding for succinate-CoA ligase as a first enzyme;
   one or more recombinant nucleic acid molecules encoding for succinyl-CoA reductase as a second enzyme; and
   one or more recombinant nucleic acid molecules encoding for 3-sulfolactaldehyde reductase or one or more recombinant nucleic acid molecules encoding for succinate semialdehyde reductase as a third enzyme,
   wherein the recombinant cell or organism is a natural producer of itaconic acid and/or comprises one or more recombinant nucleic acid molecules encoding aconitate decarboxylase, preferably aconitate decarboxylase cadA, and/or one or more recombinant nucleic acid molecules encoding for citrate synthase gltA, preferably citrate synthase gltA.
59. Recombinant cell or organisms according to item 58, wherein the recombinant cell or organism is a natural producer of itaconic acid.
60. Recombinant cell or organism according to item 58 or item 59, wherein the recombinant cell or organism comprises one or more recombinant nucleic acid molecules encoding for aconitate decarboxylase cadA and one or more recombinant nucleic acid molecules encoding for citrate synthase gltA.
61. Recombinant cell or organism according to any one of items 58-60, wherein the aconitate decarboxylase cadA is from Aspergillus terreus.
62. Recombinant cell or organism according to any one of items 58-61, wherein the aconitate decarboxylase cadA comprises an amino acid sequence with at least 70% identity to an amino acid sequence according to SEQ ID NO: 46.
63. Recombinant cell or organism according to any one of items 58-62, wherein the aconitate decarboxylase cadA comprises an amino acid sequence with at least 75%, at least 80%, at least 85%, at least 90%, at least 95%, at least 98%, at least 99% or 100% identity to an amino acid sequence according to SEQ ID NO: 46.
64. Recombinant cell or organism according to any one of items 58-63, wherein the citrate synthase gltA is from Corynebacterium glutamicum.
65. Recombinant cell or organism according to any one of items 58-64, wherein the citrate synthase gltA comprises an amino acid sequence with at least 70% identity to an amino acid sequence according to SEQ ID NO: 48.
66. Recombinant cell or organism according to any one of items 58-65, wherein the citrate synthase gltA comprises an amino acid sequence with at least 75%, at least 80%, at least 85%, at least 90%, at least 95%, at least 98%, at least 99% or 100% identity to an amino acid sequence according to SEQ ID NO: 48.
67. Recombinant cell or organism according to any one of items 58-66, wherein the recombinant cell or organism is a glucose-fermenting cell or organism.
68. Recombinant cell or organism according to any one of items 58-67, wherein the recombinant or cell organism is selected from the group consisting of Escherichia coli wild type, Gluconobacter oxydans, Streptomyces coelicolor, Streptococcus thermophiles, Pseudomonas putida, Bacillus licheniformis, Bacillus subtilis, Corynebacterium glutamicum, Pseudomonas fluorescens, Pseudomonas aeruginosa, Pseudozyma tsukubaensis, Ustilago maydis, Aspergillus niger, Aspergillus terreus, Trichoderma reesei, Pichia pastoris, Saccharomyces cerevisiae, Saccharomyces pombe and Yarrowia (candida) lipolytica, preferably, the recombinant cell or organism is Escherichia coli wild type, Escherichia coli strain Ita23, Escherichia coli Ita36A, Escherichia coli with a knock-out of glutamate-cysteine ligase (gsh), Pseudozyma tsukubaensis wild type (H488), Pseudozyma tsukubaensis strain HR12, or Pseudozyma tsukubaensis strain M15, most preferably Pseudozyma tsukubaensis strain HR12, Escherichia coli with a knock-out of glutamate-cysteine ligase (gshA), Escherichia coli Ita36A or Escherichia coli Ita36A AI.
69. Recombinant cell or organism according to any one of items 58-68, wherein the succinate-CoA ligase as a first enzyme is SucCD, and/or the succinyl-CoA reductase as a second enzyme is from Clostridium kluyveri, and/or the 3-sulfolactaldehyde reductase as a third enzyme is YihU or the succinate semialdehyde reductase as a third enzyme is AKR7A2.
70. Recombinant cell or organism according to any one of items 58-69, wherein the succinate-CoA ligase as a first enzyme is SucCD.
71. Recombinant cell or organism according to any one of items 58-69, wherein the succinyl-CoA reductase as a second enzyme is from Clostridium kluyveri.
72. Recombinant cell or organism according to any one of items 58-69, wherein the 3-sulfolactaldehyde reductase as a third enzyme is YihU.
73. Recombinant cell or organism according to any one of items 58-69, wherein the succinate semialdehyde reductase as a third enzyme is AKR7A2
74. Recombinant cell or organism according to any one of items 58-69, wherein the succinate-CoA ligase as the first enzyme is SucCD, and the succinyl-CoA reductase as the second enzyme is from Clostridium kluyveri, and the 3-sulfolactaldehyde reductase as the third enzyme is YihU.
75. Recombinant cell or organism according to any one of items 58-69, wherein the succinate-CoA ligase as the first enzyme is SucCD, and the succinyl-CoA reductase as the second enzyme is from Clostridium kluyveri, and the succinate semialdehyde reductase as the third enzyme is AKR7A2.
76. Recombinant cell or organism according to any one of items 58-75, wherein the Succinyl-CoA ligase is from Escherichia coli (SucCD, subunit beta: SucC UniProt POA836 (SEQ ID NO: 2) and subunit alpha: SucD POAGE9 (SEQ ID NO: 4)), Advenella mimigardefordensis (SucCD, subunit beta: SucC Uniprot WOPFR9 (SEQ ID NO: 6) and subunit alpha: SucD Uniprot WOPAN5 (SEQ ID NO: 8)), Alcanivorax borkumensis (SucCD, subunit beta: SucC Uniprot QOVPF7 (SEQ ID NO: 10) and subunit alpha: SucD UniProt QOVPF8 (SEQ ID NO: 12)) or Thermobifida fusca (subunit beta: Tfu_2577 Uniprot Q47LR2 (SEQ ID NO: 14) and subunit alpha: Tfu_2576 UniProt Q47LR3 (SEQ ID NO: 16)).
77. Recombinant cell or organism according to any one of items 58-76, wherein the Succinyl-CoA synthetase is from Escherichia coli.
78. Recombinant cell or organism according to any one of items 69-77, wherein the succinate-CoA ligase SucCD consists of two subunits SucC and SucD, wherein the SucC subunit comprises an amino acid sequence with at least 70% identity to an amino acid sequence according to SEQ ID NO: 2 and wherein the SucD subunit comprises an amino acid sequence with at least 70% identity to an amino acid sequence according to SEQ ID NO: 4.
79. Recombinant cell or organism according to any one of items 69-78, the succinate-CoA ligase SucCD consists of two subunits SucC and SucD, wherein subunit SucC of the Succinyl-CoA synthetase SucCD is at least 75%, at least 80%, at least 85%, at least 90%, at least 95%, at least 98%, at least 99% or 100% identical with an amino acid sequence according to SEQ ID NO: 2 and subunit SucD of the Succinyl-CoA synthetase SucCD is at least 75%, at least 80%, at least 85%, at least 90%, at least 95%, at least 98%, at least 99% or 100% identical with an amino acid sequence according to SEQ ID NO: 4.
80. Recombinant cell or organism according to any one of items 69-79, wherein the succinyl-CoA reductase Scr is from Clostridium kluyveri.
81. Recombinant cell or organism according to any one of items 69-80, wherein the succinyl-CoA reductase comprises an amino acid sequence with at least 70% identity to an amino acid sequence according to SEQ ID NO: 18.
82. Recombinant cell or organism according to any one of items 69-81, wherein the succinyl-CoA reductase is at least 75%, at least 80%, at least 85%, at least 90%, at least 95%, at least 98%, at least 99% or 100% identical with an amino acid sequence according to SEQ ID NO: 18.
83. Recombinant cell or organism according to any one of items 58-72, 74, and 76-82, wherein the third enzyme is 3-sulfolactaldehyde reductase YihU from Escherichia coli strain K12.
84. Recombinant cell or organism according to any one of items 58-72, 74, and 76-83, wherein the third enzyme is 3-sulfolactaldehyde reductase YihU and comprises an amino acid sequence with at least 70% identity to an amino acid sequence according to SEQ ID NO: 30.
85. Recombinant cell or organism according to any one of items 58-72, 74, and 76-84, wherein the third enzyme is 3-sulfolactaldehyde reductase YihU and comprises an amino acid sequence with at least 75%, at least 80%, at least 85%, at least 90%, at least 95%, at least 98%, at least 99% or 100% identity to an amino acid sequence according to SEQ ID NO: 30.
86. Recombinant cell or organism according to any one of items 58-71, 73, and 75-82, wherein the third enzyme is succinate semialdehyde reductase and is from Homo sapiens.
87. Recombinant cell or organism according to any one of items 58-71, 73, and 75-82, and 86, wherein the third enzyme is succinate semialdehyde reductase and comprises an amino acid sequence with at least 70% identity to an amino acid sequence according to SEQ ID NO: 40.
88. Recombinant cell or organism according to any one of items 58-71, 73, and 75-82, and 86-87, wherein the third enzyme is succinate semialdehyde reductase and comprises an amino acid sequence with at least 75%, at least 80%, at least 85%, at least 90%, at least 95%, at least 98%, at least 99% or 100% identity to an amino acid sequence according to SEQ ID NO: 40.
89. Recombinant cell or organism according to any one of items 58-71, 73, and 75-82, and 86-88, wherein the third enzyme is succinate semialdehyde reductase and is AKR7A2.
90. Recombinant cell or organism according to any one of items 58-72, 74, and 76-85, wherein
   the succinate-CoA ligase as the first enzyme is SucCD and consists of two subunits SucC and SucD, wherein the SucC subunit comprises an amino acid sequence with at least 70% identity to an amino acid sequence according to SEQ ID NO: 2 and wherein the SucD subunit comprises an amino acid sequence with at least 70% identity to an amino acid sequence according to SEQ ID NO: 4; and
   the succinyl-CoA reductase as the second enzyme is from Clostridium kluyveri and comprises an amino acid sequence with at least 70% identity to an amino acid sequence according to SEQ ID NO: 18, and
   the 3-sulfolactaldehyde reductase as the third enzyme is YihU and comprises an amino acid sequence with at least 70% identity to an amino acid sequence according to SEQ ID NO: 30.
91. Recombinant cell or organism according to any one of items 58-71, 73, and 75-82, and 86-89, wherein
   the succinate-CoA ligase as the first enzyme is SucCD and consists of two subunits SucC and SucD, wherein the SucC subunit comprises an amino acid sequence with at least 70% identity to an amino acid sequence according to SEQ ID NO: 2 and wherein the SucD subunit comprises an amino acid sequence with at least 70% identity to an amino acid sequence according to SEQ ID NO: 4; and
   the succinyl-CoA reductase as the second enzyme is from Clostridium kluyveri and comprises an amino acid sequence with at least 70% identity to an amino acid sequence according to SEQ ID NO: 18; and
   the succinate semialdehyde reductase as the third enzyme comprises an amino acid sequence with at least 70% identity to an amino acid sequence according to SEQ ID NO: 40.
92. Recombinant cell or organism according to any one of items 58-91, wherein said recombinant cell or organism comprises one or more recombinant nucleic acid molecules encoding for an acyl transferase as a fourth enzyme.
93. Recombinant cell or organism according to item 92, wherein the acyl transferase has an amino acid sequence selected from the group consisting of SEQ ID NO: 32, SEQ ID NO: 34, SEQ ID NO: 36, SEQ ID NO: 38, SEQ ID NO: 42, SEQ ID NO: 44 and SEQ ID NO: 50.
94. Recombinant cell or organism according to any one of items 58-91, wherein said recombinant cell or organism comprises one or more recombinant nucleic acid molecules encoding for a glycosyltransferase as a fourth enzyme.
95. Recombinant cell or organism according to item 94, wherein the glycosyltransferase is a UDP-glycosyltransferase.
96. Recombinant cell or organism according to item 95, wherein the UDP-glycosyltransferase has the amino acid sequence according to SEQ ID NO: 60.
97. Recombinant cell or organism according to any one of items 94 to 96, wherein the glycosyltransferase comprises a tag which enhances solubility of the glycosyltransferase in the recombinant cell or organism.
98. Recombinant cell or organism according to item 97, wherein the tag which enhances solubility of the glycosyltransferase in the recombinant cell or organism is selected from the group consisting of: Maltose binding protein, Strep-Tag, SUMO-Tag, Trx-Tag, NusA-Tag, GST-Tag, SET-Tag, DsbC-Tag, Skp-Tag, T7PK-Tag, GB1-Tag and ZZ-Tag.
99. Recombinant cell or organism according to any one of items 58-98, wherein the recombinant cell or organism produces at least 14 UM tulipalin A in 40 h, preferably at least 25 μM tulipalin A in 50 h.
100. Recombinant cell or organism according to any one of items 58-99, wherein the recombinant cell or organism expresses reduced levels of endogenous aldehyde reductases compared to wild-type endogenous levels.
101. Recombinant cell or organism according to item 100, wherein the recombinant cell or organism with reduced aldehyde reductase expression is Escherichia coli strain K12 MG1655.
102. Use of a recombinant cell or organism according to any one of items 58-101 for the in vivo production of tulipalin A (α-methylene-γ-butyrolactone).
103. A method for producing tulipalin A (α-methylene-γ-butyrolactone) from itaconic acid, the method comprising the steps of:
   (a) culturing at least one recombinant cell or organism selected from bacteria and fungi in a fermentation medium, said recombinant cell or organism comprising
      one or more recombinant nucleic acid molecules encoding for succinate-CoA ligase as a first enzyme;
      one or more recombinant nucleic acid molecules encoding for succinyl-CoA reductase as a second enzyme; and
      one or more recombinant nucleic acid molecules encoding for 3-sulfolactaldehyde reductase or one or more recombinant nucleic acid molecules encoding for succinate semialdehyde reductase as a third enzyme;
   (b) harvesting the at least one recombinant cell or organism;
   (c) lysing the at least one recombinant cell or organism to prepare a lysate;
   (d) incubating the lysate with itaconic acid under suitable conditions, preferably at a temperature of 25° C.-40° C. for six to 48 hours, more preferably at a temperature of 30° C. for six hours; and
   (e) isolating the tulipalin A.
104. A method for producing tulipalin A (α-methylene-γ-butyrolactone) from itaconic acid, the method comprising the steps of:
   (a) culturing in a fermentation medium:
      a first recombinant cell or organism selected from bacteria and fungi, said recombinant cell or organism comprising one or more recombinant nucleic acid molecules encoding for succinate-CoA ligase as a first enzyme;
      a second recombinant cell or organism selected from bacteria and fungi, said recombinant cell or organism comprising one or more recombinant nucleic acid molecules encoding for succinyl-CoA reductase as a second enzyme; and
      a third recombinant cell or organism selected from bacteria and fungi, said recombinant cell or organism comprising one or more recombinant nucleic acid molecules encoding for 3-sulfolactaldehyde reductase or one or more recombinant nucleic acid molecules encoding for succinate semialdehyde reductase as a third enzyme;
   (b) harvesting the first, second and third recombinant cell or organism;
   (c) lysing the the first, second and third recombinant cell or organism to prepare a first, second and third lysate;
   (d) combining the first, second and third lysate in a suitable ratio, preferably in the ratio 1:1:1, thereby producing a lysate mixture;
   (d) incubating the lysate mixture with itaconic acid under suitable conditions, preferably at a temperature of 25° C.-40° C. for six to 48 hours, more preferably at a temperature of 30° C. for six hours; and
   (e) isolating the tulipalin A.
105. Method according to any one of items 103 and 104, wherein the recombinant cell or organism is a glucose-fermenting cell or organism.
106. Method according to any one of items 103-105, wherein the recombinant or cell organism is selected from the group consisting of Escherichia coli wild type, Gluconobacter oxydans, Streptomyces coelicolor, Streptococcus thermophiles, Pseudomonas fluorescens, Pseudomonas aeruginosa, Pseudomonas putida, Bacillus licheniformis, Bacillus subtilis, Corynebacterium glutamicum, Pseudozyma tsukubaensis, Ustilago maydis, Aspergillus niger, Aspergillus terreus, Trichoderma reesei, Pichia pastoris, Saccharomyces cerevisiae, Saccharomyces pombe Ashbya gossypii and Yarrowia (candida) lipolytica, preferably, the recombinant cell or organism is Escherichia coli wild type, Escherichia coli strain Ita23, Escherichia coli Ita36A and related strains such as Ita36A AI, Escherichia coli with a knock-out of glutamate-cysteine ligase (gshA), Pseudozyma tsukubaensis wild type (H488), Pseudozyma tsukubaensis strain HR12, or Pseudozyma tsukubaensis strain M15, most preferably Pseudozyma tsukubaensis strain HR12, Escherichia coli with a knock-out of glutamate-cysteine ligase (gshA), Escherichia coli Ita36A or Escherichia coli Ita36A AI.
107. Method according to any one of items 103-106, wherein the succinate-CoA ligase as a first enzyme is SucCD, and/or the succinyl-CoA reductase as a second enzyme is from Clostridium kluyveri, and/or the 3-sulfolactaldehyde reductase as a third enzyme is YihU or the succinate semialdehyde reductase as a third enzyme is AKR7A2.
108. Method according to any one of items 103-107, wherein the succinate-CoA ligase as a first enzyme is SucCD.
109. Method according to any one of items 103-107, wherein the succinyl-CoA reductase as a second enzyme is Scr.
110. Method according to any one of items 103-107, wherein the 3-sulfolactaldehyde reductase as a third enzyme is YihU.
111. Method according to any one of items 103-107, wherein the succinate semialdehyde reductase as a third enzyme is AKR7A2.
112. Method according to item 108, wherein the succinyl-CoA reductase as a second enzyme is Scr.
113. Method according to item 108 or item 112, wherein the 3-sulfolactaldehyde reductase as a third enzyme is YihU.
114. Method according to item 108 or item 112, wherein the succinate semialdehyde reductase as a third enzyme is AKR7A2.
115. Method according to any one of items 103-107, wherein the succinate-CoA ligase as the first enzyme is SucCD, and the succinyl-CoA reductase as the second enzyme is from Clostridium kluyveri, and the 3-sulfolactaldehyde reductase as the third enzyme is YihU.
116. Method according to any one of items 103-107, wherein the succinate-CoA ligase as the first enzyme is SucCD, and the succinyl-CoA reductase as the second enzyme is from Clostridium kluyveri, and the succinate semialdehyde reductase as the third enzyme is AKR7A2.
117. Method according to any one of items 103-116, wherein the Succinyl-CoA synthetase is from Escherichia coli (SucCD, subunit beta: SucC UniProt POA836 (SEQ ID NO: 2) and subunit alpha: SucD POAGE9 (SEQ ID NO: 4)), Advenella mimigardefordensis (SucCD, subunit beta: SucC Uniprot WOPFR9 (SEQ ID NO: 6) and subunit alpha: SucD Uniprot WOPAN5 (SEQ ID NO: 8)), Alcanivorax borkumensis (SucCD, subunit beta: SucC Uniprot QOVPF7 (SEQ ID NO: 10) and subunit alpha: SucD UniProt QOVPF8 (SEQ ID NO: 12)) or Thermobifida fusca (subunit beta: Tfu_2577 Uniprot Q47LR2 (SEQ ID NO: 14) and subunit alpha: Tfu_2576 UniProt Q47LR3 (SEQ ID NO: 16)).
118. Method according to any one of items 103-117, wherein the Succinyl-CoA synthetase is from Escherichia coli.
119. Method according to any one of items 107-118, wherein the succinate-CoA ligase SucCD consists of two subunits SucC and SucD, wherein the SucC subunit comprises an amino acid sequence with at least 70% identity to an amino acid sequence according to SEQ ID NO: 2 and wherein the SucD subunit comprises an amino acid sequence with at least 70% identity to an amino acid sequence according to SEQ ID NO: 4.
120. Method according to any one of items 107-119, wherein the succinate-CoA ligase SucCD consists of two subunits SucC and SucD, wherein subunit SucC of the Succinyl-CoA synthetase SucCD is at least 75%, at least 80%, at least 85%, at least 90%, at least 95%, at least 98%, at least 99% or 100% identical with an amino acid sequence according to SEQ ID NO: 2 and subunit SucD of the Succinyl-CoA synthetase SucCD is at least 75%, at least 80%, at least 85%, at least 90%, at least 95%, at least 98%, at least 99% or 100% identical with an amino acid sequence according to SEQ ID NO: 4.
121. Method according to any one of items 107 to 120, wherein the succinyl-CoA reductase is from Clostridium kluyveri.
122. Method according to any one of items 107 to 121, wherein the succinyl-CoA reductase comprises an amino acid sequence with at least 70% identity to an amino acid sequence according to SEQ ID NO: 18.
123. Method according to any one of items 107 to 122, wherein the succinyl-CoA reductase is at least 75%, at least 80%, at least 85%, at least 90%, at least 95%, at least 98%, at least 99% or 100% identical with an amino acid sequence according to SEQ ID NO: 18.
124. Method according to any one of items 103-113, 115, and 117-123, wherein the third enzyme is 3-sulfolactaldehyde reductase YihU from Escherichia coli strain K12.
125. Method according to any one of items 103-113, 115, and 117-124, wherein the third enzyme is 3-sulfolactaldehyde reductase YihU and comprises an amino acid sequence with at least 70% identity to an amino acid sequence according to SEQ ID NO: 30.
126. Method according to any one of items 103-113, 115, and 117-125, wherein the third enzyme is 3-sulfolactaldehyde reductase YihU and comprises an amino acid sequence with at least 75%, at least 80%, at least 85%, at least 90%, at least 95%, at least 98%, at least 99% or 100% identity to an amino acid sequence according to SEQ ID NO: 30.
127. Method according to any one of items 103-112, 114, and 116-123, wherein the third enzyme is succinate semialdehyde reductase and is from Homo sapiens.
128. Method according to any one of items 103-112, 114, 116-123 and 127, wherein the third enzyme is succinate semialdehyde reductase and comprises an amino acid sequence with at least 70% identity to an amino acid sequence according to SEQ ID NO: 40.
129. Method according to any one of items 103-112, 114, 116-123 and 127-128, wherein the third enzyme is succinate semialdehyde reductase and comprises an amino acid sequence with at least 75%, at least 80%, at least 85%, at least 90%, at least 95%, at least 98%, at least 99% or 100% identity to an amino acid sequence according to SEQ ID NO: 40.
130. Method according to any one of items 103-112, 114, 116-123 and 127-129, wherein the third enzyme is succinate semialdehyde reductase and is AKR7A2.
131. Method according to any one of items 103-113, 115, and 117-126, wherein
   the succinate-CoA ligase as the first enzyme is SucCD and consists of two subunits SucC and SucD, wherein the SucC subunit comprises an amino acid sequence with at least 70% identity to an amino acid sequence according to SEQ ID NO: 2 and wherein the SucD subunit comprises an amino acid sequence with at least 70% identity to an amino acid sequence according to SEQ ID NO: 4; and
   the succinyl-CoA reductase as the second enzyme is from Clostridium kluyveri and comprises an amino acid sequence with at least 70% identity to an amino acid sequence according to SEQ ID NO: 18, and
   the 3-sulfolactaldehyde reductase as the third enzyme is YihU and comprises an amino acid sequence with at least 70% identity to an amino acid sequence according to SEQ ID NO: 30.
132. Method according to any one of items 103-112, 114, 116-123 and 127-130, wherein
   the succinate-CoA ligase as the first enzyme is SucCD and consists of two subunits SucC and SucD, wherein the SucC subunit comprises an amino acid sequence with at least 70% identity to an amino acid sequence according to SEQ ID NO: 2 and wherein the SucD subunit comprises an amino acid sequence with at least 70% identity to an amino acid sequence according to SEQ ID NO: 4; and
   the succinyl-CoA reductase as the second enzyme is from Clostridium kluyveri and comprises an amino acid sequence with at least 70% identity to an amino acid sequence according to SEQ ID NO: 18, and
   the succinate semialdehyde reductase as the third enzyme comprises an amino acid sequence with at least 70% identity to an amino acid sequence according to SEQ ID NO: 40.
133. Method according to any one of items 103-132, wherein said recombinant cell or organism comprises one or more recombinant nucleic acid molecules encoding for an acyl transferase as a fourth enzyme.
134. Method according to item 133, wherein the acyl transferase has an amino acid sequence selected from the group consisting of SEQ ID NO: 32, SEQ ID NO: 34, SEQ ID NO: 36, SEQ ID NO: 38, SEQ ID NO: 42, SEQ ID NO: 44 and SEQ ID NO: 50.
135. Method according to any one of items 103-134, wherein said recombinant cell or organism comprises one or more recombinant nucleic acid molecules encoding for a glycosyltransferase as a fourth enzyme.
136. Method according to item 135, wherein the glycosyltransferase is a UDP-glycosyltransferase.
137. Method according to item 136, wherein the UDP-glycosyltransferase has the amino acid sequence according to SEQ ID NO: 60.
138. Method according to any one of items 135 to 137, wherein the glycosyltransferase comprises a tag which enhances solubility of the glycosyltransferase in the recombinant cell or organism.
139. Method according to item 138, wherein the tag which enhances solubility of the glycosyltransferase in the recombinant cell or organism is selected from the group consisting of: Maltose binding protein, Strep-Tag, SUMO-Tag, Trx-Tag, NusA-Tag, GST-Tag, SET-Tag, DsbC-Tag, Skp-Tag, T7PK-Tag, GB1-Tag and ZZ-Tag.

### EXAMPLES

The following examples are provided for illustrative purposes. It is thus understood that the examples are not to be construed as limiting. The skilled person will clearly be able to envisage further modifications of the principles laid out herein.

### Materials and Methods

Itaconic acid and Tulipalin A were purchased from Sigma Aldrich (Munich, Germany). Chemicals and materials used for the protein expression were purchased from New England Biolabs GmbH (Frankfurt am Main, Germany), Macharey-Nagel GmbH (Düren, Germany) and GE Healthcare. Identity of all the recombinant proteins was confirmed using SDS-PAGE.

### Enzymes

Enzymes were isolated from the source organism and cloned into plasmid vectors as indicated in Table 1. Table 1 provides the enzyme name, full name, source organism, UniProt accession number, vector and SEQ ID NO of enzymes used in the examples and enzymes useful for the invention.

TABLE 1
Exemplary enzymes used in the invention

| | | | UniProt/ | | |
|---|---|---|---|---|---|
| Enzyme | Name | Source | Genbank | Vector | SEQ ID NO: |
| SucC | ADP-forming | Escherichia coli | P0A836 | pPW136 | 1: gene |
| | succinate-CoA | (strain K12) | | | 2: protein |
| | ligase | | | | 3: gene |
| | subunit beta | | | | 4: protein |
| SucD | ADP-forming | | P0AGE9 | | |
| | succinate-CoA | | | | |
| | ligase | | | | |
| | subunit alpha | | | | |
| SucC | ADP-forming | Advenella | W0PFR9 | | 5: gene |
| | succinate-CoA | mimigardefordensis | | | 6: protein |
| | ligase | | | | 7: gene |
| | subunit beta | | | | 8: protein |
| SucD | ADP-forming | | W0PAN5 | | |
| | succinate-CoA | | | | |
| | ligase | | | | |
| | subunit alpha | | | | |
| SucC | ADP-forming | Alcanivorax | Q0VPF7 | | 9: gene A |
| | succinate-CoA | borkumensis | | | 10: protein A |
| | ligase | | | | 11: gene B |
| | subunit beta | | | | 12: protein B |
| SucD | ADP-forming | | Q0VPF8 | | |
| | succinate-CoA | | | | |
| | ligase | | | | |
| | subunit alpha | | | | |
| Scr | Succinyl-CoA | Clostridium kluyveri | P38947 | pPW136 | 17: gene |
| | reductase | | | | 18: protein |
| Scr | Succinate | Methylobacterium | A0A2U8VWW1 | | 27: gene |
| | semialdehyde | radiodurans | | | 28: protein |
| | dehydrogenase | | | | |
| YihU | 3- | Escherichia coli | P0A9V8 | pPW136 | 29: gene |
| | sulfolactaldehyde | (strain K12) | | | 30: protein |
| | reductase | | | | |
| AKR7A2 | Succinate | Homo sapiens | O43488 | | 39: gene |
| | semialdehyde | | | | 40: protein |
| | dehydrogenase | | | | |
| CadA | aconitate | Aspergillus terreus | | pCadCS | 45: gene |
| | decarboxylase | | | | 46: protein |
| GltA | citrate synthase | Corynebacterium | | pCadCS | 47: gene |
| | | glutamicum | | | 48: protein |

### Plasmids Containing the Pathway to Tulipalin a Via SucCD, Scr, and YihU or AKR7A2

Production of plasmid pPW136 for E. coli: Gene SucCD consists of two subunits SucC and SucD. The two subunits were amplified from E. coli MG1655 with the native RBS of SucD. The genes were cloned under the control of a pBAD promoter. Genes Scr (succinyl-CoA reductase) and YihU were cloned with a T7 promoter. Spectinomycin is used for selection of the plasmid. The nucleic acid sequence of plasmid pPW136 is shown in SEQ ID NO: 51.

Production of plasmid pPW157 for E. coli: Gene SucCD consists of two subunits SucC and SucD. The two subunits were amplified from E. coli MG1655 with the native RBS of SucD. The genes were cloned under the control of a pBAD promoter. Genes Scr (succinyl-CoA reductase) and AKR7A2 were cloned with a T7 promoter. Spectinomycin is used for selection of the plasmid. The nucleic acid sequence of plasmid pPW157 is shown in SEQ ID NO: 52.

Production of the plasmid for P. tsukubaensis: Gene SucCD consists of two subunits, SucC and SucD. The two subunits were cloned on the same plasmid with hygromycin as selection marker for P. tsukubaensis. The selection marker hygromycin was cloned under the control of the HSP70 promoter and HSP70 terminator, SucC under the control of the ACT1 promoter and TEF1 terminator and SucD under the control of GAPDH promoter and HSP70 terminator. Scr was cloned under the control of the TEF1 promoter and GAPDH terminator. YihU was cloned under the control of the Actin promoter and HSP70 terminator. The selection marker carboxin was cloned under the control of the HSP70 promoter and HSP70 terminator.

Plasmid pCadCS. Plasmid Containing the Enzymes for the Pathway to Itaconic Acid Via cadA and gltA.

Production of plasmid pCadCS is described in Harder et al. (“Model-based metabolic engineering enables high yield itaconic acid production by Escherichia coli”, Metabolic Engineering, 2016, 38, p. 29-37).

### Transformation Protocol

Transformation of E. coli

E. coli strains with or without plasmid were cultivated in 10 mL LB or TB (with respective antibiotics) overnight at 37° C. 1 mL of the overnight culture was harvested and washed 3× in 10% ice-cold glycerol to make them electrocompetent. About 100 μL of culture resuspended in 10% glycerol was transferred to a pre-chilled 1 mm electroporation cuvette. Plasmid was added to the cells in the cuvette and electroporation was performed using program Ec1 on a biorad MicroPulser. LB was added to the cells and they were rescued for 1-1.5 h at 37° C. Afterwards the cells were plated on selective LB agar plates.

Transformation of P. tsukubaensis

P. tsukubaensis cells were cultivated in 20 ml YPD overnight at 160 rpm. The overnight culture adjusted to OD=1 was used to inoculate 50 ml YPD and cultivated at 30° C. for 6 h. The cells were centrifuged, washed in sterile water and suspended in 2 ml protoplast buffer containing 50 mg Vinotaste. The mixture was incubated at 30° C. for 10 min that resulted in protoplast formation. The cells were further washed in 5 ml cold SCS twice followed by 5 ml STC. The washed pellet was eventually resuspended in 0.5 ml STC for transformation. 2-5 μg plasmid was then added to the 80 μl of the protoplast cells and incubated in ice for 10 min. 500 μl PEG solution was added and the mixture was further incubated in ice for 10 min. To this, 1 ml YPD, 1 M sorbitol and 2% glucose was added and incubated at 30° C. for 20 min. After a brief centrifugation, the cells were once again resuspended in 1 ml YPD, 1 M sorbitol and 2% glucose and left at 30° C. overnight. The overnight cells were plated on YPD agar plates and further left at 30° C. until colonies appear.

### Example 1

Cloning (E. coli) and Strain Construction (E. coli)

All constructs for E. coli were cloned using Golden Gate and the Ecoflex Kit in accordance with the manufacturer's protocol. The E. coli strain ita36A (Harder et al., 2018) has a synthetic genetic control in one of TCA cycle genes. The promoter of the isocitrate dehydrogenase was replaced by the lambda promoter, the expression being controlled by means of a temperature-sensitive repressor that becomes active at temperatures lower than 30° C. At regular growth temperature of 37° C., the cells rely are relying on the TCA cycle for their growth, which is decoupled from the itaconate production phase at lower temperature 28° C. To make it compatible with the T7-promoter based system, a modified version of this strain ita36A AI (AI=Arabinose Inducible) was generated by integration of T7-RNA polymerase into the araB locus of ita36A which was cured of the pCadCS plasmid, using P1 transduction with E. coli BL21AI as a donor. Tetracycline resistance was used as a selection marker. The integration was confirmed via PCR and Sanger sequencing. The expression of the enzymes was confirmed by Western Blot.

### Production of Tulipalin A In Vivo

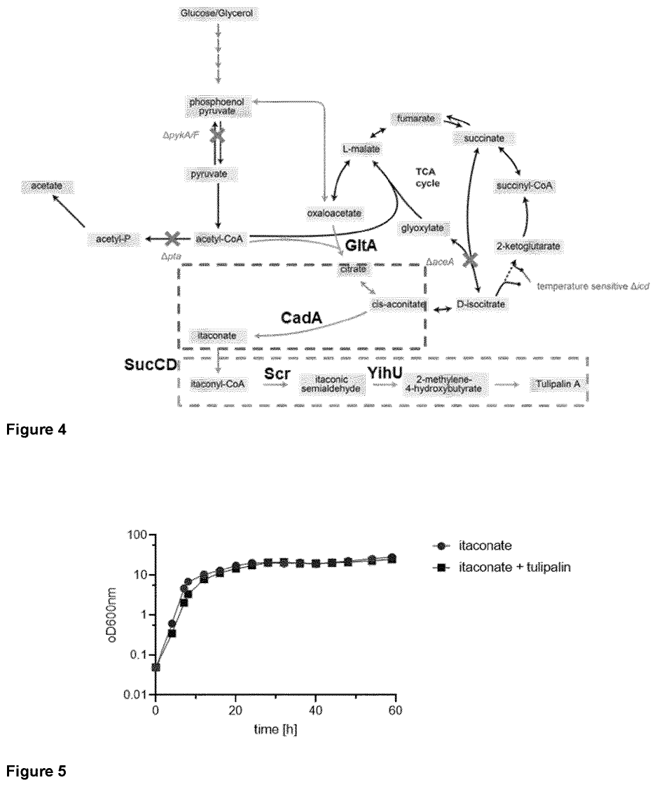

Tulipalin A was produced in E. coli strain ita36A AI harboring plasmids for the itaconate production pathway pCadCS and the tulipalin production pathway pPW136. The overall metabolic scheme is depicted in FIG. 4. Cells were transformed via electroporation with the plasmids and plated on LB agar plates with the respective antibiotics or double antibiotics selecting for the plasmid. A single resulting transformant was inoculated in 10 mL TB with Kanamycin, Spectinomycin and Tetracycline to select for plasmids and the strain with the T7 RNA-polymerase. Negative controls are biological triplicates of strain ita36A AI only with the plasmid pCadCS. They were treated the same way, however no spectinomycin was included in the media. The cells were grown overnight at 37° C. and then diluted to an OD600 nm 0.05 in 200 mL TB. The cells were grown for 7 h at 37° C., 130 rpm in baffled flasks with sampling outlet. After 7 h, the cells were shifted to 28° C., induced with 0.025% (w/v) L-Arabinose and 500 UM IPTG. Upon induction 50 mM glycerol was added to the media. Every 12 h from the start of the cultivation, 100 mM glycerol was added to the media. At different timepoints, 1 mL samples from 3 biological replicates were taken and 200 μL were filtered with a 0.22 μM hydrophilic MultiScreen-GV plate (Milipore) to obtain the supernatant samples. The samples were stored at −20° C. until analysis. To enable quantification of tulipalin A and itaconic acid, the supernatant was acidified to 0.1% HCl and used for LC-MS/MS analysis as described below. The growth pattern of 3 biological replicates each is depicted in FIG. 5.

### LC-MS/MS Analysis

Quantitative determination of itaconic acid and tulipalin A was performed using a LC-MS/MS. The chromatographic separation was performed on an Agilent Infinity II 1290 HPLC system using a Kinetex EVO C18 column (150×1.7 mm, 1.7 μm particle size, 100 Å pore size, Phenomenex) connected to a guard column of similar specificity (20×2.1 mm, sub 2 μm particle size, Phenomenex) at a constant flow rate of 0.15 ml/min with mobile phase A being 0.1% formic acid in water and phase B being 0.1% formic acid in methanol (Honeywell, Morristown, New Jersey, USA) at 40° C. The injection volume was 1 μl. The mobile phase profile consisted of the following steps and linear gradients: 0-7 min 5 to 100% B; 7-9 min constant at 100% B; 9-9.1 min from 100 to 5% B; 9.1-15 min constant at 5% B. An Agilent 6495 ion funnel mass spectrometer was used in positive and negative mode with an electrospray ionization source and the following conditions: ESI spray voltage 2000 V, nozzle voltage 500 V, sheath gas 400° C. at 11 l/min, nebulizer pressure 50 psig and drying gas 80° C. at 16 l/min. Compounds were identified based on their mass transition and retention time compared to standards. Chromatograms were integrated using MassHunter software (Agilent, Santa Clara, CA, USA). Absolute concentrations were calculated based on an external calibration curve prepared in sample matrix. Mass transitions, collision energies, Cell accelerator voltages and Dwell times have been optimized using chemically pure standards. Parameter settings of all targets are given in Table 2.

TABLE 2
Parameter settings for LC-MS/MS

| | | | | | Collision | Cell | |
|---|---|---|---|---|---|---|---|
| | Precursor | Product | | Fragmentor | energy | Accelerator | |
| Compound | Ion | Ion | Dwell | (V) | (V) | (V) | Polarity |
| Itaconic | 129 | 129 | 70 | 380 | 0 | 5 | Negative |
| acid | | | | | | | |
| Itaconic | 129 | 185 | 70 | 380 | 6 | 5 | Negative |
| acid | | | | | | | |
| Tulipalin A | 99 | 81 | 70 | 380 | 8 | 5 | Positive |
| Tulipalin A | 99 | 53 | 70 | 380 | 14 | 5 | Positive |

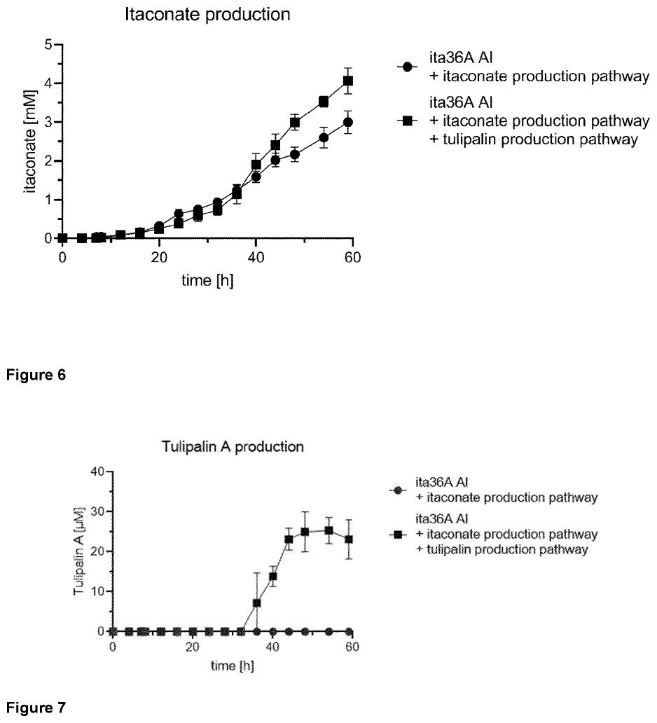

The results of this quantification of itaconic acid and tulipalin A are depicted in FIGS. 6 and 7.

### Example 2

Cloning (E. coli) and Strain Construction (E. coli)

All constructs for E. coli were cloned using Golden Gate and the Ecoflex Kit in accordance with the manufacturer's protocol. The E. coli strain ita36A (Harder et al., 2018) has a synthetic genetic control in one of TCA cycle genes. The promoter of the isocitrate dehydrogenase was replaced by the lambda promoter, the expression being controlled by means of a temperature-sensitive repressor that becomes active at temperatures lower than 30° C. At regular growth temperature of 37° C., the cells rely are relying on the TCA cycle for their growth, which is decoupled from the itaconate production phase at lower temperature 28° C. To make it compatible with the T7-promoter based system, a modified version of this strain ita36A AI (AI=Arabinose Inducible) was generated by integration of T7-RNA polymerase into the araB locus of ita36A which was cured of the pCadCS plasmid, using P1 transduction with E. coli BL21AI as a donor. Tetracycline resistance was used as a selection marker. The integration was confirmed via PCR and Sanger sequencing. The expression of the enzymes was confirmed by Western Blot.

### Production of Tulipalin A In Vivo

Tulipalin A was produced in E. coli strain ita36A AI harboring plasmids for the itaconate production pathway pCadCS and the tulipalin production pathway pPW157. Cells were transformed via electroporation with the plasmids and plated on LB agar plates with the respective antibiotics or double antibiotics selecting for the plasmid. A single resulting transformant was inoculated in 10 mL TB with Kanamycin, Spectinomycin and Tetracycline to select for plasmids and the strain with the T7 RNA-polymerase. Negative controls are biological triplicates of strain ita36A AI only with the plasmid pCadCS. They were treated the same way, however no spectinomycin was included in the media. The cells were grown overnight at 37° C. and then diluted to an OD₆₀₀ₙₘ 0.05 in 20 mL TB. The cells were grown for 7 h at 37° C., 130 rpm in baffled flasks with sampling outlet. After 7 h, the cells were shifted to 28° C., induced with 0.025% (w/v) L-Arabinose and 500 UM IPTG. Upon induction 50 mM glycerol was added to the media. 24 and 48 hours after start of the cultivation, 100 mM glycerol was added to the media. Tulipalin A was extracted using equal amounts of ethylacetate and media (including cells). The extract was used for analysis.

### HPLC Analysis

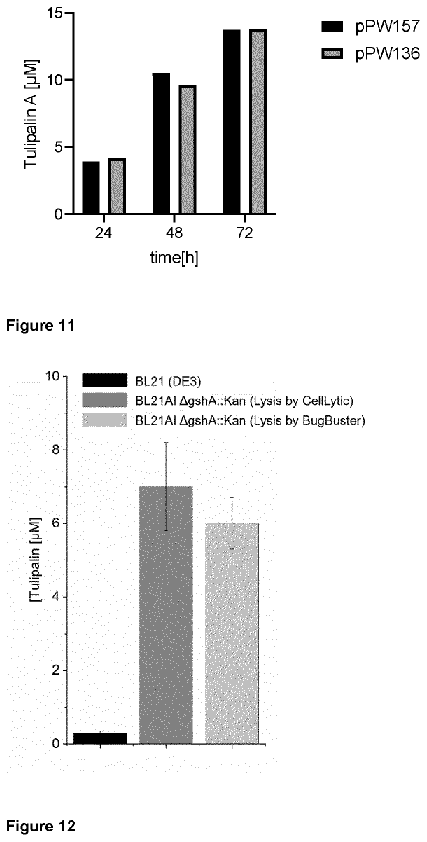

Quantitative determination of tulipalin A from ethylacetate extracts was performed using an Agilent 1290 Infinity II UHPLC with a ZORBAX SB-C18 (Analytical 4.6×50 mm 5 UM, Agilent) and DAD. A constant flow rate of 1 mL/min with mobile phase A being 0.1% TFA in water and mobile phase B being acetonitrile+0.1% TFA at 40° C. was used. Either 3 μl or 20 μL were injected. The gradient consisted of the following steps: 0.1-10 min 5% B, 10-12 min 7.5% B, 12-14 min 100% B, 14-16 min 100% B, 16-18 min 5% B, 19-20 5% B. The compounds were detected at the following wavelengths: tulipalin A 211.6 nm. Compounds were identified based on their retention time of standards in matrix. Results are depicted in FIG. 11.

### Example 3: Cell-Free Production of Tulipalin A with Enzymes Produced in Glutathione Deficient E. coli Cells

Generation of E. coli BL21AI ΔgshA::Kan

E. coli BL21AI ΔgshA::Kan was generated using P1 transduction as described in Thomason et al. (2014) Current Protocols in Molecular Biology 79: 1.17.1-1.17.8 with a gshA knock-out strain from the KEIO collection (Baba et al. (2006) Mol. Syst. Biol. 2:2006.0008). The knockout was confirmed via colony PCR and subsequent Sanger-Sequencing.

Production of proteins in the E. coli BL21AI ΔgshA::Kan cells Production of plasmid pPW185 for E. coli: The two subunits SucC and SucD of gene SucCD were amplified from E. coli MG1655 with the native RBS of SucD and cloned under the control of a T7 promoter. The genes Scr (succinyl-CoA reductase) and AKR7A2 were cloned with a T7 promoter. Cis-aconitate decarboxylase and citrate synthase were amplified from plasmid pCadCS and cloned with promoter J23100 and TL2 RBS (Moore et al. (2016) ACS Synth. Biol. 5 (10): 1059-1069). Spectinomycin was used for selection of the plasmid. The nucleic acid sequence of plasmid pPW185 encoding for SucCD, Scr, AKRA7A2, cis-aconitate dehydrogenase and citrate synthase is shown in SEQ ID NO: 63.

BL21AI ΔgshA:Kan was transformed with plasmid pPW185 using electroporation. Cells were washed 3× in ice cold 10% glycerol and then transferred to a pre-chilled 1 mm electroporation cuvette and pre-set Ec1 (Biorad) was used for electroporation. Immediately after electroporation, 1 mL of LB or SOC was added, and the cells were incubated for 1 h at 37° C. before inoculating them in 600 μl autoinducing medium (according to Studier, supplemented with 0.025% (w/v) L-Arabinose for BL21AI cells) and appropriate antibiotics in 96 well plates. The cells were grown for 24 h at 25° C. Afterwards, cells were harvested (2,000×g, 10 min) and resuspended in 60 μl of CelLytic (Sigma-Aldrich B7435) or BugBuster (Merck 70584-M). Higher lysis was achieved by shaking at 25° C. for another 15 mins. The lysate was mixed to a final concentration of 1:5 in 100 mM HEPES pH 7.5, 10 mM MgCl2, 50 mM Glucose, 50 mM itaconate and chloramphenicol (34 μg/mL). The reaction was run for 6 h at 30° C. and quenched with 1% HCl and washed with brine. The reaction was extracted with equal volume of ethyl acetate, centrifuged for 1 min at 2,0000×g and the supernatant was analysed with LC-MS according to the below protocol.

As a control, plasmids encoding for SucCD (pNO218, SEQ ID NO: 64), Scr (pPW155, SEQ ID NO: 65) and AKR7A2 (pPW150, SEQ ID NO: 66) were produced separately in E. coli BL21 (DE3) which was transformed with the respective plasmids heat shock. Cells were thawed on ice and 1 μL of plasmid DNA was added and icubated for 10-30 minutes. Afterwards, the cells were heat shocked for 45 seconds at a temperature of 42° C. and then placed on ice. 1 mL of LB or SOC was added, and the cells were incubated for 1 h at 37° C. before plating on LB plates with respective antibiotics. To produce the proteins, the cells were inoculated in autoinducing medium (according to Studier)) and appropriate antibiotics. The cells were grown for 24 h at 25° C. Afterwards, cells were harvested and resuspended in 5 ml/g pellet with 50 mM HEPES pH 7.5, 500 mM KCl and lysed twice using an Avestin Emulsifier B-12. The lysates were mixed in a 1:1:1 ratio to a final concentration of 1:5 in 100 mM HEPES pH 7.5, 10 mM MgCl2, 50 mM Glucose, 50 mM itaconate and chloramphenicol (34 μg/mL). The reaction was run for 6 h at 30° C. and quenched with 1% HCl and washed with brine. The reaction was extracted with equal volume of ethyl acetate, centrifuged for 1 min at 2,0000×g and the supernatant was analysed with LC-MS as described below.

The chromatographic separation was performed on an Agilent Infinity II 1290 HPLC system using a Kinetex EVO C18 column (50×2.1 mm, 3 μm particle size, 100 Å pore size, Phenomenex) connected to a guard column of similar specificity (20×2.1 mm, 3 μm particle size, Phenomoenex) at a constant flow rate of 0.2 mL/min with mobile phase A being 0.1% formic acid in water and phase B being 0.1% formic acid in methanol (Honeywell, Morristown, New Jersey, USA) at 40° C.

The injection volume was 0.5 μL.

The profile of the mobile phase consisted of the following steps and linear gradients: 5-2.5 min from 0% to 100% B; 2.5-3.4 min constant at 100% B; 3.5-3.6 min from 100% to 0% B; 3.6-7 min constant at 5% B.

An Agilent 6470 mass spectrometer was used in positive and negative mode with an electrospray ionization source and the following conditions: ESI spray voltage 4500 V, nozzle voltage 500 V, sheath gas 300° C. at 11 L/min, nebulizer pressure 45 psig and drying gas 170° C. at 5 L/min.

Compounds were identified based on their mass transition and retention time compared to standards.

Chromatograms were integrated using MassHunter software (Agilent, Santa Clara, CA, USA). Relative abundance was determined based on the peak area. Absolute concentrations were determined based on an external Standard curve.

Mass transitions, collision energies, Cell accelerator voltages and Dwell times have been optimized using chemically pure standards. Parameter settings of all targets are given in the table below

| | | | Collision | Fragmentor | Cell | Dwell | |
|---|---|---|---|---|---|---|---|
| | Precursor | Product | energy | Voltage | Accelerator | time | |
| Name | Ion | Ion | [V] | [V] | Voltage [V] | [msec] | Polarity |
| Tulipalin A | 99 | 99 | 0 | 85 | 5 | 150 | Positive |
| Tulipalin A | 99 | 81 | 8 | 85 | 5 | 150 | Positive |
| Tulipalin A | 99 | 53 | 14 | 85 | 5 | 150 | Positive |
| Itaconic | 129 | 129 | 0 | 70 | 5 | 150 | Negative |
| Acids | | | | | | | |
| Itaconic Acid | 129 | 85 | 6 | 70 | 5 | 150 | Negative |

Results are depicted in FIG. 12.

### Example 4: Production of Tuliposide A Using UDP-Glycosyltransferase

UDP Glycosyltransferase tagged with His-Tag and MBP (Maltose Binding Protein) (see SEQ ID NO: 62) was produced in E. coli BL21 (DE3), purified using Ni-NTA agarose and desalted in desalting buffer (50 mM HEPES pH 7.5, 150 mM KCl) afterwards. Enzyme activity was tested in a Cary60 spectrophotometer at 30° C. The formation of UDP was monitored using PK-LDH (pyruvatkinase-lactate dehydrogenase) at 340 nm. Briefly, 100 mM HEPES pH 7.5, 10 mM MgCl2, 1 μm of UDP-Glycosyltransferase, 1 mM PEP (phosphoenolpyruvate), 1 μl PK-LDH Mix (Sigma P0294), 0.25 mM NADH, 1 mM UDP-Glucose and varying amounts of 2-Methylene-4-hydroxybutyrate were tested. Activity was calculated using Lambert-Beer's Law with ε_{NADH340nm}=6.22 mM⁻¹ cm⁻¹. Results are depicted in FIG. 14.

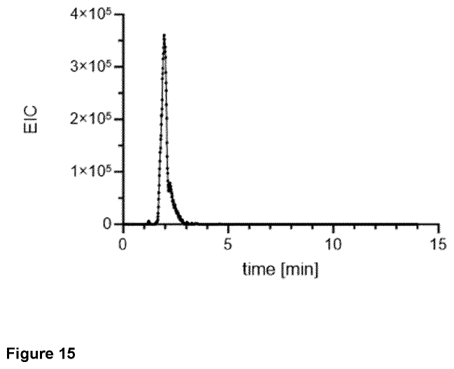

Tuliposide formation was confirmed via HPLC-ESI-TOF on a 6550 iFunnel Q-TOF LC-MS (Agilent) in negative mode with a 1.8-μm Zorbax SB-C18 column, 50×2.1 mm (Agilent) and using H₂O (buffer A) and acetonitrile (buffer B) both containing 0.1% formic acid. The gradient conditions were as follows: 0 min 80% B, 8 min 60% B, 10 min 10% B, 12 min 10% B and 14 min 80% B with a flow rate of 300 μl min⁻¹. Capillary voltage was set at 4 kV, and nitrogen gas was used as nebulizing (60 psig), drying (11 l min⁻¹, 100° C.) and sheath (12 l min⁻¹, 250° C.) gas. MS data were acquired with a scan range of 50-1,700 m/z. Data were analyzed using MassHunter Analysis software (Agilent). Results are depicted in FIG. 15.

1.-19. (canceled)

20. An in vivo method for producing tulipalin A (α-methylene-γ-butyrolactone) from itaconic acid, the method comprising culturing at least one recombinant cell or organism selected from bacteria and fungi in a fermentation medium, said recombinant cell or organism comprising:
one or more recombinant nucleic acid molecules encoding for succinate-CoA ligase as a first enzyme;
one or more recombinant nucleic acid molecules encoding for succinyl-CoA reductase as a second enzyme; and
one or more recombinant nucleic acid molecules encoding for 3-sulfolactaldehyde reductase or one or more recombinant nucleic acid molecules encoding for succinate semialdehyde reductase as a third enzyme.

21. The method according to claim 20, wherein the recombinant cell or organism
is a natural producer of itaconic acid; and/or
comprises one or more recombinant nucleic acid molecules encoding for aconitate decarboxylase cadA and/or one or more recombinant nucleic acid molecules encoding for citrate synthase gltA.

22. The method according to claim 20, wherein the fermentation medium comprises
CaCO₃, preferably at least 3 g/L CaCO₃, more preferably at least 5 g/L CaCO₃, even more preferably at least 10 g/L CaCO₃; and/or
at least 0.1 g/L phosphate, preferably at least 0.2 g/L phosphate, more preferably at least 0.4 g/L phosphate source, wherein the phosphate source is preferably selected from KH₂PO₄ and K₂HPO₄, and mixtures thereof, preferably an equivalent mixture thereof.

23. The method according to claim 20, further comprising the step of the step of isolating tulipalin A, wherein the isolating preferably encompasses the step of extracting tulipalin A from the fermentation medium by means of at least one organic solvent, preferably wherein the extracting is continuous.

24. The method according to claim 23, wherein the organic solvent
is not harmful to the recombinant cell or organism; and/or
is selected from the group consisting of 2-tert-butylphenol, ethyl acetate, hexyl acetate, butyl acetate, 4-methyl-2-pentanon, cyclohexanol, dodecane, isopropyl myristate, n-hexane, n-heptane, oleyl alcohol, bis(2-ethylhexyl) phthalate (DEHP), ethyl oleate, tridecan, n-octanol, 1-hexanol, methyl tert-butyl ether, and mixtures thereof, preferably is 2-tert-butylphenol, ethyl acetate, hexyl acetate, butyl acetate, 4-methyl-2-pentanon or cyclohexanol.

25. The method according to claim 23, wherein the extracting is performed at a pH in the range of 4 to 9, preferably in the range of 5 to 8.

26. A recombinant cell or organism, said recombinant cell or organism being selected from bacteria and fungi and comprising:
one or more recombinant nucleic acid molecules encoding for succinate-CoA ligase as a first enzyme;
one or more recombinant nucleic acid molecules encoding for succinyl-CoA reductase as a second enzyme; and
one or more recombinant nucleic acid molecules encoding for 3-sulfolactaldehyde reductase or one or more recombinant nucleic acid molecules encoding for succinate semialdehyde reductase as a third enzyme;
wherein the recombinant cell or organism is
(a) a natural producer of itaconic acid and/or
(b) comprises one or more recombinant nucleic acid molecules encoding for cis-aconitate decarboxylase, preferably aconitate decarboxylase cadA, and/or one or more recombinant nucleic acid molecules encoding for citrate synthase gltA, preferably citrate synthase gltA.

27. The recombinant cell or organism according to claim 26, wherein the recombinant cell or organism is a glucose-fermenting cell or organism.

28. The recombinant cell or organism according to claim 26, wherein the recombinant or cell organism is selected from the group consisting of Escherichia coli wild type, Gluconobacter oxydans, Streptomyces coelicolor, Streptococcus thermophiles, Pseudomonas fluorescens, Pseudomonas aeruginosa, Pseudomonas putida, Bacillus licheniformis, Bacillus subtilis, Corynebacterium glutamicum, Pseudozyma tsukubaensis, Ustilago maydis, Aspergillus niger, Aspergillus terreus, Trichoderma reesei, Pichia pastoris, Saccharomyces cerevisiae, Saccharomyces pombe, Ashbya gossypii and Yarrowia (candida) lipolytica, preferably, the recombinant cell or organism is Escherichia coli wild type, Escherichia coli strain Ita23, Escherichia coli Ita36A and related strains such as Ita36A AI, Escherichia coli with a knock-out of the glutamate-cysteine ligase (gsh) gene, Pseudozyma tsukubaensis wild type (H488), Pseudozyma tsukubaensis strain HR12, or Pseudozyma tsukubaensis strain M15, most preferably the recombinant cell or organism is Pseudozyma tsukubaensis strain HR12, Escherichia coli with a knock-out of the glutamate-cysteine ligase (gsh) gene, Escherichia coli Ita36A or Escherichia coli Ita36A AI.

29. The recombinant cell or organism according to claim 26, wherein
the succinate-CoA ligase as a first enzyme is SucCD; and/or
the succinyl-CoA reductase as a second enzyme is from Clostridium kluyveri; and/or
the 3-sulfolactaldehyde reductase as a third enzyme is YihU or the succinate semialdehyde reductase as a third enzyme is AKR7A2.

30. The recombinant cell or organism according to claim 26, wherein succinate-CoA ligase SucCD
is from Escherichia coli; and/or
consists of two subunits SucC and SucD, wherein the SucC subunit comprises an amino acid sequence with at least 70% identity to an amino acid sequence according to SEQ ID NO: 2 and wherein the SucD subunit comprises an amino acid sequence with at least 70% identity to an amino acid sequence according to SEQ ID NO: 4.

31. The recombinant cell or organism according to claim 26, wherein succinyl-CoA reductase comprises an amino acid sequence with at least 70% identity to an amino acid sequence according to SEQ ID NO: 18.

32. The recombinant cell or organism according to claim 26, wherein the 3-sulfolactaldehyde reductase YihU as the third enzyme
is from Escherichia coli strain K12; and/or
comprises an amino acid sequence with at least 70% identity to an amino acid sequence according to SEQ ID NO: 30.

33. The recombinant cell or organism according to claim 26, wherein the succinate semialdehyde reductase as the third enzyme
is from Homo sapiens; and/or
comprises an amino acid sequence with at least 70% identity to an amino acid sequence according to SEQ ID NO: 40.

34. The recombinant cell or organism according to claim 26, wherein cis-aconitate decarboxylase cadA
is from Aspergillus terreus; and/or
comprises an amino acid sequence with at least 70% identity to an amino acid sequence according to SEQ ID NO: 46.

35. The recombinant cell or organism according to claim 26, wherein citrate synthase gltA
is from Corynebacterium glutamicum; and/or
comprises an amino acid sequence with at least 70% identity to an amino acid sequence according to SEQ ID NO: 48.

36. Use of a recombinant cell or organism according to claim 26 for the in vivo production of tulipalin A (α-methylene-γ-butyrolactone).

37. A method for producing tulipalin A (α-methylene-γ-butyrolactone) from itaconic acid, the method comprising the steps of:
(a) culturing at least one recombinant cell or organism selected from bacteria and fungi in a fermentation medium, said recombinant cell or organism comprising
one or more recombinant nucleic acid molecules encoding for succinate-CoA ligase as a first enzyme;
one or more recombinant nucleic acid molecules encoding for succinyl-CoA reductase as a second enzyme; and
one or more recombinant nucleic acid molecules encoding for 3-sulfolactaldehyde reductase or one or more recombinant nucleic acid molecules encoding for succinate semialdehyde reductase as a third enzyme;
(b) harvesting the at least one recombinant cell or organism;
(c) lysing the at least one recombinant cell or organism to prepare a lysate;
(d) incubating the lysate with itaconic acid under suitable conditions; and
(e) isolating the tulipalin A.

38. A method for producing tulipalin A (α-methylene-γ-butyrolactone) from itaconic acid, the method comprising the steps of:
(a) culturing in a fermentation medium:
a first recombinant cell or organism selected from bacteria and fungi, said recombinant cell or organism comprising one or more recombinant nucleic acid molecules encoding for succinate-CoA ligase as a first enzyme;
a second recombinant cell or organism selected from bacteria and fungi, said recombinant cell or organism comprising one or more recombinant nucleic acid molecules encoding for succinyl-CoA reductase as a second enzyme; and
a third recombinant cell or organism selected from bacteria and fungi, said recombinant cell or organism comprising one or more recombinant nucleic acid molecules encoding for 3-sulfolactaldehyde reductase or one or more recombinant nucleic acid molecules encoding for succinate semialdehyde reductase as a third enzyme;
(b) harvesting the first, second and third recombinant cell or organism;
(c) lysing the first, second and third recombinant cell or organism to prepare a first, second and third lysate;
(d) combining the first, second and third lysate in a suitable ratio, thereby producing a lysate mixture;
(e) incubating the lysate mixture with itaconic acid under suitable conditions; and
(f) isolating the tulipalin A.


SEQUENCE LISTING (66 sequences)

SEQ ID NO: 1 - DNA, 1167 bp, Escherichia coli
  source 1..1167: mol_type = genomic DNA
gccaacttacatgaatatcaggcaaaacaactttttgcccgctatggcttaccagcaccggtgggttatgcctgtactactccgcgcgaagcagaagaagccgcttcaaaaatcggtgccggtccgtgggtagtgaaatgtcaggttcacgctggtggccgcggtaaagcgggcggtgtgaaagttgtaaacagcaaagaagacatccgtgcttttgcagaaaactggctgggcaagcgtctggtaacgtatcaaacagatgccaatggccaaccggttaaccagattctggttgaagcagcgaccgatatcgctaaagagctgtatctcggtgccgttgttgaccgtagttcccgtcgtgtggtctttatggcctccaccgaaggcggcgtggaaatcgaaaaagtggcggaagaaactccgcacctgatccataaagttgcgcttgatccgctgactggcccgatgccgtatcagggacgcgagctggcgttcaaactgggtctggaaggtaaactggttcagcagttcaccaaaatcttcatgggcctggcgaccattttcctggagcgcgacctggcgttgatcgaaatcaacccgctggtcatcaccaaacagggcgatctgatttgcctcgacggcaaactgggcgctgacggcaacgcactgttccgccagcctgatctgcgcgaaatgcgtgaccagtcgcaggaagatccgcgtgaagcacaggctgcacagtgggaactgaactacgttgcgctggacggtaacatcggttgtatggttaacggcgcaggtctggcgatgggtacgatggacatcgttaaactgcacggcggcgaaccggctaacttccttgacgttggcggcggcgcaaccaaagaacgtgtaaccgaagcgttcaaaatcatcctctctgacgacaaagtgaaagccgttctggttaacatcttcggcggtatcgttcgttgcgacctgatcgctgacggtatcatcggcgcggtagcagaagtgggtgttaacgtaccggtcgtggtacgtctggaaggtaacaacgccgaactcggcgcgaagaaactggctgacagcggcctgaatattattgcagcaaaaggtctgacggatgcagctcagcaggttgttgccgcagtggaggggaaataa

SEQ ID NO: 2 - protein, 388 aa, Escherichia coli
ANLHEYQAKQLFARYGLPAPVGYACTTPREAEEAASKIGAGPWVVKCQVHAGGRGKAGGVKVVNSKEDIRAFAENWLGKRLVTYQTDANGQPVNQILVEAATDIAKELYLGAVVDRSSRRVVFMASTEGGVEIEKVAEETPHLIHKVALDPLTGPMPYQGRELAFKLGLEGKLVQQFTKIFMGLATIFLERDLALIEINPLVITKQGDLICLDGKLGADGNALFRQPDLREMRDQSQEDPREAQAAQWELNYVALDGNIGCMVNGAGLAMGTMDIVKLHGGEPANFLDVGGGATKERVTEAFKIILSDDKVKAVLVNIFGGIVRCDLIADGIIGAVAEVGVNVPVVVRLEGNNAELGAKKLADSGLNIIAAKGLTDAAQQVVAAVEGK

SEQ ID NO: 3 - DNA, 870 bp, Escherichia coli
  source 1..870: mol_type = genomic DNA
gcctccattttaatcgataaaaacaccaaggttatctgccagggctttaccggtagccaggggactttccactcagaacaggccattgcatacggcactaaaatggttggcggcgtaaccccaggtaaaggcggcaccacccacctcggcctgccggtgttcaacaccgtgcgtgaagccgttgctgccactggcgctaccgcttctgttatctacgtaccagcaccgttctgcaaagactccattctggaagccatcgacgcaggcatcaaactgattatcaccatcactgaaggcatcccgacgctggatatgctgaccgtgaaagtgaagctggatgaagcaggcgttcgtatgatcggcccgaactgcccaggcgttatcactccgggtgaatgcaaaatcggtatccagcctggtcacattcacaaaccgggtaaagtgggtatcgtttcccgttccggtacactgacctatgaagcggttaaacagaccacggattacggtttcggtcagtcgacctgtgtcggtatcggcggtgacccgatcccgggctctaactttatcgacattctcgaaatgttcgaaaaagatccgcagaccgaagcgatcgtgatgatcggtgagatcggcggtagcgctgaagaagaagcagctgcgtacatcaaagagcacgttaccaagccagttgtgggttacatcgctggtgtgactgcgccgaaaggcaaacgtatgggccacgcgggtgccatcattgccggtgggaaagggactgcggatgagaaattcgctgctctggaagccgcaggcgtgaaaaccgttcgcagcctggcggatatcggtgaagcactgaaaactgttctgaaataa

SEQ ID NO: 4 - protein, 289 aa, Escherichia coli
ASILIDKNTKVICQGFTGSQGTFHSEQAIAYGTKMVGGVTPGKGGTTHLGLPVFNTVREAVAATGATASVIYVPAPFCKDSILEAIDAGIKLIITITEGIPTLDMLTVKVKLDEAGVRMIGPNCPGVITPGECKIGIQPGHIHKPGKVGIVSRSGTLTYEAVKQTTDYGFGQSTCVGIGGDPIPGSNFIDILEMFEKDPQTEAIVMIGEIGGSAEEEAAAYIKEHVTKPVVGYIAGVTAPKGKRMGHAGAIIAGGKGTADEKFAALEAAGVKTVRSLADIGEALKTVLK

SEQ ID NO: 5 - DNA, 1161 bp, Advenella mimigardefordensis
  source 1..1161: mol_type = genomic DNA
atgaaaattcacgaataccagggtaaggaacttctcaagaagtttggagtcacagtaccacgtgggattccagccttcagtgtcgatgaggctgtggcggctgcggaaaagttaggcgggccagtgtgggtcgtgaaagcacaaatccacgccggcgggcgcgggaaaggtggtggtgttaaattagcgaaatcgatcgatgaggtacgccaactgtccagtgagatccttggtatgcaattagttacccatcaaactggaccggaaggccaaaaggttaatcgcctcttaatcgaggaaggtgcagatattaagaaagagtattatgttggcattgttactgaccgttcaagtcaacgtgtttgtgtcatggcaagctctgagggtggtatggatgtggaagaggtcgcagaaaagacgcctgagaaaatcttaaaggtattcgtagacccggcgaagggcttcactgacgacgaggcgcgtgagcttgcccgcggaattggggtgcctgatgcgagtgtggaaaaggcggcggtcgagttccagaagctctaccaagcatattgggacaccgattcgagcttagcagagattaatccgttaatcctcactggaagcggcgacattatcgcattggatgcgaagtttaatttcgatagtaatgcgcttttccgccatcctgagattgtggaatatcgtgacttagatgaagaagatccggcggagattgaggcatctaagttcgacttagcatatatccaattagatggaaacattgggtgtcttgtgaatggcgcaggcctggcaatggctacgatggacactattaaacttttcggtggggagccggcgaattttcttgacgttggcggcggggccacggccgagaaagtaactgaggcgtttaaaattatgctgaagaataaagaggttaaggcaatcttggttaacatcttcggtgggattatgcgttgtgacgtaattgcagagggtgttattgccgcgtgtaaggcggtgaatctgtcagtgccactcgtcgtccgtatgaaaggcactaacgaggaaatggggaagaagatgttggcagaatcgggtctgccgattatttcggcagacactatggccgaggctgccaccaaggttgtcgcagcagtgaaatga

SEQ ID NO: 6 - protein, 386 aa, Advenella mimigardefordensis
MKIHEYQGKELLKKFGVTVPRGIPAFSVDEAVAAAEKLGGPVWVVKAQIHAGGRGKGGGVKLAKSIDEVRQLSSEILGMQLVTHQTGPEGQKVNRLLIEEGADIKKEYYVGIVTDRSSQRVCVMASSEGGMDVEEVAEKTPEKILKVFVDPAKGFTDDEARELARGIGVPDASVEKAAVEFQKLYQAYWDTDSSLAEINPLILTGSGDIIALDAKFNFDSNALFRHPEIVEYRDLDEEDPAEIEASKFDLAYIQLDGNIGCLVNGAGLAMATMDTIKLFGGEPANFLDVGGGATAEKVTEAFKIMLKNKEVKAILVNIFGGIMRCDVIAEGVIAACKAVNLSVPLVVRMKGTNEEMGKKMLAESGLPIISADTMAEAATKVVAAVK

SEQ ID NO: 7 - DNA, 882 bp, Advenella mimigardefordensis
  source 1..882: mol_type = genomic DNA
atgtcaattctgatcaataaagactccaaagtcatcactcagggtattaccggtaaaaccggccagttccacacacgtatgtgccgtgattacgccaatggtaaagaagcttttgttgctggtgtgaatcctaaacgcgcaggcgaggatttcgaaggcgtgcctatttacgcttctgtcaaggacgcgaaagaacaaacgggcgccaatgtatctgttatctatgttccaccagcaggtgccgctgatgccatctgggaagcggtagacgctgacctggatctggtgatctgcattaccgaaggtattcctgttcgcgatatgctggctgtccgcaaccgcatgcgccaggaaaacaaaaaaacactgctgctgggccctaactgccctggtctgattacgcccgatgagctgaaaatcggcattatgccagcgcacatctgcaaaaaaggccgcattggtgtggtcagccgttcaggcacgcttacatacgaagccgttgcccagttgacggagctcggcctgggacaatccagtgcagttggtattggtggtgacccgatcaatggtctcaaacacatcgatatcctgaaaatgttcaacgacgatccagagaccgatgcggttgtgatgattggtgagatcggcgggccggacgaagtcaatgccgccgaatgggccaaagacaatatgagcaagccagtagtgggctttatcgccggtgttactgctcctccaggaaaacgcatgggccacgccggtgcgctgatctccggtggcgccgatacggccgatgccaagctggaaatcatggaagcttgcggtatccgtaccacacgcaatccatctgaaatgggcaaactgctcaaatcagtactgtaa

SEQ ID NO: 8 - protein, 293 aa, Advenella mimigardefordensis
MSILINKDSKVITQGITGKTGQFHTRMCRDYANGKEAFVAGVNPKRAGEDFEGVPIYASVKDAKEQTGANVSVIYVPPAGAADAIWEAVDADLDLVICITEGIPVRDMLAVRNRMRQENKKTLLLGPNCPGLITPDELKIGIMPAHICKKGRIGVVSRSGTLTYEAVAQLTELGLGQSSAVGIGGDPINGLKHIDILKMFNDDPETDAVVMIGEIGGPDEVNAAEWAKDNMSKPVVGFIAGVTAPPGKRMGHAGALISGGADTADAKLEIMEACGIRTTRNPSEMGKLLKSVL

SEQ ID NO: 9 - DNA, 1170 bp, Alcanivorax borkumensis
  source 1..1170: mol_type = genomic DNA
atgaacctgcatgaatatcagagcaaacagctgttcgcagattatggtctgccggttagtaccggcttcgcctgcgataccccggaagaagttgccgcaaaagccaaagaaattggcggcgataaatgggttgtgaaagcacaggtgcacgctggcggccgtggtaaagccggcggtgttaaactggtggataccccggaggatgcagcagccttcgcaaaaaaatggctgggtaaacagctggttaccttccagaccgatgaacatggtcagccggttagcaaaattctggttgaaacctgcaccgatattgccaaagaactgtatctgagcgccgtgctggatcgcgccagccgtcgtgttgtgttcatggccagtaccgaaggtggtgtggatattgaaaccgtggcagaagaaacacctgaaaaaattctgaaagccgaaattgatccgctggttggcgcacagccgtatcaggcacgcgaaattgcattcaaactgggtctggaaggcaaacaggttaatcagttcgccaaaatcttcgtgggtctggccaaactgttcgttgataaagatctggccctgattgaagtgaatccgctggttattaccgataccggtgatctgcattgcctggatgccaaaattaatgtggatggtagtgccctgtatcgccatccggatattaaagcactggatgatccgagccaggaagatgaacgcgaacgtcgtgccgcagaatgggatctgaattatgttgccctggaaggtaatattggctgcatggttaatggcgcaggtctggcaatgggcacaatggatctggttaaactgaaaggcggcgccccggcaaacttcctggatgttggcggcggtgcaaccaaagaacgcgttaccgaagcattcaaaattattctgagcgatgatcaggtgaaaggtgtgctggttaatatcttcggcggtattgttcgttgtgatctgattgccgaaggcattattggtgcagttgaagaagttggtgttaccgttccggtggtggttcgcctggaaggtaacaatgccgaactgggcagtcagaaactggccgaaagtggcatgaatattattgcagcccagagcttcgatgatgccgccgaacaggttgtgaaagcggtgggcggcgcagcctaa

SEQ ID NO: 10 - protein, 389 aa, Alcanivorax borkumensis
MNLHEYQSKQLFADYGLPVSTGFACDTPEEVAAKAKEIGGDKWVVKAQVHAGGRGKAGGVKLVDTPEDAAAFAKKWLGKQLVTFQTDEHGQPVSKILVETCTDIAKELYLSAVLDRASRRVVFMASTEGGVDIETVAEETPEKILKAEIDPLVGAQPYQAREIAFKLGLEGKQVNQFAKIFVGLAKLFVDKDLALIEVNPLVITDTGDLHCLDAKINVDGSALYRHPDIKALDDPSQEDERERRAAEWDLNYVALEGNIGCMVNGAGLAMGTMDLVKLKGGAPANFLDVGGGATKERVTEAFKIILSDDQVKGVLVNIFGGIVRCDLIAEGIIGAVEEVGVTVPVVVRLEGNNAELGSQKLAESGMNIIAAQSFDDAAEQVVKAVGGAA

SEQ ID NO: 11 - DNA, 879 bp, Alcanivorax borkumensis
  source 1..879: mol_type = genomic DNA
atgggcagtgtgctgattgataaaaacaccaaagttatctgtcagggcttcaccggcagccagggcaccctgcatagtgaacaggccattgaatatggtacccagatggtgggcggtgtgacccctggtaaaggcggccagacccatctgggtctgccggtgttcaataccgtggcagaagccgttgccgaaaccggcgccaccgccagtgttatctatgtgccggcaccgttctgtaaagatagtattctggaagcagcagatgcaggcattgaactgattgtgtgtattaccgaaggcgttccggcactggatatgctgttcgttaaagaatatattagccgtaaagacggcattcgcctgattggtccgaattgtccgggtgttattacccctggtgaatgtaaaattggcattatgccgggccatattcatacccctggtaaggtgggtattgtgagccgcagcggcaccctgacctatgaagcagtgaatcagaccaccaaactgggcttcggtcagagtagttgcattggcattggcggtgatccgattccgggcatgaccttcattgatgccctggaactgttccagaatgatccgcagaccgaagcaattgtgatggtgggcgaaattggtggtaccgccgaagatgaagcagcagcctatattaaagaacatgttaccaaaccggtggttagttatattgcaggcgtgaccgccccggccggcaaaagaatgggccatgccggcgccattattgcaggcggtaaaggcaccgcagatgaaaaattcgccgttctggaagcagcgggcgtgaaaaccgtgcgcaatccggccgaaattggcaaagccgtgaaagaagcaaccggttggtaa

SEQ ID NO: 12 - protein, 292 aa, Alcanivorax borkumensis
MGSVLIDKNTKVICQGFTGSQGTLHSEQAIEYGTQMVGGVTPGKGGQTHLGLPVFNTVAEAVAETGATASVIYVPAPFCKDSILEAADAGIELIVCITEGVPALDMLFVKEYISRKDGIRLIGPNCPGVITPGECKIGIMPGHIHTPGKVGIVSRSGTLTYEAVNQTTKLGFGQSSCIGIGGDPIPGMTFIDALELFQNDPQTEAIVMVGEIGGTAEDEAAAYIKEHVTKPVVSYIAGVTAPAGKRMGHAGAIIAGGKGTADEKFAVLEAAGVKTVRNPAEIGKAVKEATGW

SEQ ID NO: 13 - DNA, 1182 bp, Thermobifida fusca
  source 1..1182: mol_type = genomic DNA
atggatttatttgagtatcaagctaaagcgctgtttgctgagtacggcgtgccagtgcctcagggcaaagttgcaagtactccggaagaagtccgcgcaattgcggaagaatttgcagccgccggtaaaccgcgtgttgttgtgaaggcgcaagttaaaactggtggacgcggtaaagcaggtggagtgaaagttgcagatgggccggatgatgcggttgcgaaagcgaaacaaatcttaggtatggatattaaaggtcatactgtacatcgtgttttggtagaggaagcgtccgatattgcggaagaatattatttctcgtttcttttggatcgcgccaatcgcagctttctgagcatttgttcagcggaaggcggcatggaaattgaagaagtggccgccacgaatcctgatgctgtcgcaaaagtaccgatttccccattaaaaggcgcacctgcagatgtggcagcagatattgtggcgcagggtaagttgccggaagccgccgcgcaaggagcggtggatgtgattactaaattgtggaaggtgtttgtcgaaaaggatgcgaccttagttaatgtgaatcctctgattttgacgaaagatgggcgtgtagtggcattagatggaaaagttactctcgatgataatgcagaatttcgtcaagatttagaaagcctggcgagcgcagcagagggagatcctttagaggtgaaagcgaaagagaaaggtctgaattatgtaaaactggatggcgaagtgggtattattggcaatggggcgggcctggttatgagtaccttggatgtagttgcgtatgcgggcgaacaacatggtggtgtaaaaccggcgaattttctggatattggtggtggtgcgagtgcggaagtgatggcgaatggtttagaaattattttgtcagatccggccgttaaatcggtatttgttaatgtctttggcggcattacggcctgtgatgcagtggcgaatggaattgtccaagctttagagctcttggagtcgcgtggagaagacgttacaaaaccacttgttgtccgtcttgatggcaataatgcagagcttgggcgttctattctgaatgagcgcaatcatccggcagttcgtcaagtagatacgatggatggtgccgcggcgctggctgcagagttagcggctaaatga

SEQ ID NO: 14 - protein, 393 aa, Thermobifida fusca
MDLFEYQAKALFAEYGVPVPQGKVASTPEEVRAIAEEFAAAGKPRVVVKAQVKTGGRGKAGGVKVADGPDDAVAKAKQILGMDIKGHTVHRVLVEEASDIAEEYYFSFLLDRANRSFLSICSAEGGMEIEEVAATNPDAVAKVPISPLKGAPADVAADIVAQGKLPEAAAQGAVDVITKLWKVFVEKDATLVNVNPLILTKDGRVVALDGKVTLDDNAEFRQDLESLASAAEGDPLEVKAKEKGLNYVKLDGEVGIIGNGAGLVMSTLDVVAYAGEQHGGVKPANFLDIGGGASAEVMANGLEIILSDPAVKSVFVNVFGGITACDAVANGIVQALELLESRGEDVTKPLVVRLDGNNAELGRSILNERNHPAVRQVDTMDGAAALAAELAAK

SEQ ID NO: 15 - DNA, 879 bp, Thermobifida fusca
  source 1..879: mol_type = genomic DNA
atggcgatctttcttactaaggacagtaaggtattagtacagggcatgaccggtagcgagggcactaagcacacccgtcgcatgttagctgccggaactaacatcgttggtggcgtcaaccctcgcaaggcagggcaggtggttgacttcgacggcacccaggtcccagtattcggatcggtcgcagagggtatgaaggctaccggggcggacgttacagttattttcgttccgccgaagttcgcgaaggacgcagtgatcgaagcgatcgacgcagagatcggcttagccgtcgtgatcaccgaaggtatcccggtgcacgacaccgccacgttctgggcccacgcgtgctcaaagggtaacaagacccgcatcatcgggcccaactgccctggacttatcactcctggtcagtccaacgcgggcatcatccctgccgacatcaccaagccgggccgtatcggtctggtgagcaagtcgggtactctgacctaccagatgatgtacgagctgcgtgacattggtttctccacctgcgttggcatcggcggagaccccatcatcggcaccactcacatcgacgcgctcgccgcgttcgaagctgaccctgacaccgacgtcatcgtgatgatcggtgagatcggtggtgacgctgaagaacgcgccgccgaatacatcaagaagcacgtaaccaagccggtagtcggctacatcgcgggcttcaccgcgcctgagggtaagaccatgggacatgccggcgcaattgttagcggatccagtggtaccgccgcggcaaagaaggaagcattagaggctgtcggggtcaaggtaggtaagaccccgagtgaggctgcgaagttagttcgtagtctgttctaa

SEQ ID NO: 16 - protein, 292 aa, Thermobifida fusca
MAIFLTKDSKVLVQGMTGSEGTKHTRRMLAAGTNIVGGVNPRKAGQVVDFDGTQVPVFGSVAEGMKATGADVTVIFVPPKFAKDAVIEAIDAEIGLAVVITEGIPVHDTATFWAHACSKGNKTRIIGPNCPGLITPGQSNAGIIPADITKPGRIGLVSKSGTLTYQMMYELRDIGFSTCVGIGGDPIIGTTHIDALAAFEADPDTDVIVMIGEIGGDAEERAAEYIKKHVTKPVVGYIAGFTAPEGKTMGHAGAIVSGSSGTAAAKKEALEAVGVKVGKTPSEAAKLVRSLF

SEQ ID NO: 17 - DNA, 1362 bp, Clostridium kluyveri
  source 1..1362: mol_type = genomic DNA
atgagtaatgaagtatctataaaagaattaattgaaaaggcaaaggtggcacaaaaaaaattggaagcctatagtcaagaacaagttgatgtactagtaaaagcactaggaaaagtggtttatgataatgcagaaatgtttgcaaaagaagcagttgaagaaacagaaatgggtgtttatgaagataaagtagctaaatgtcatttgaaatcaggagctatttggaatcatataaaagacaagaaaactgtaggcataataaaagaagaacctgaaagggcacttgtttatgttgctaagccaaagggagttgtggcagctactacgcctataactaatccagtggtaactcctatgtgtaatgcaatggctgctataaagggcagaaatacaataatagtagcaccacatcctaaagcaaagaaagtttcagctcatactgtagaacttatgaatgctgagcttaaaaaattgggagcaccagaaaatatcatacagatagtagaagcaccatcaagagaagctgctaaggaacttatggaaagtgctgatgtagttattgctacaggcggtgctggaagagttaaagctgcttactccagtggaagaccagcttatggcgttggacctggaaattcacaggtaatagttgataagggatacgattataacaaagctgcacaggatataataacaggaagaaaatatgacaatggaattatatgttcttcagagcaatcagttatagctcctgctgaagattatgataaggtaatagcagcttttgtagaaaatggggcattctatgtagaagatgaggaaacagtagaaaagtttagatcaactttatttaaagatggaaaaataaacagcaagattataggtaaatccgtccaaattattgcggatcttgcaggagtaaaagtaccagaaggtactaaggttatagtacttaagggtaaaggtgcaggagaaaaagatgtactttgtaaagaaaaaatgtgtccagttttagtagcattgaaatatgatacttttgaagaagcagttgaaatagctatggctaattatatgtatgaaggagctggtcatacagcaggcatacattctgacaatgacgagaacataagatatgcaggaactgtattacctataagcagattagttgtaaatcagcctgcaactactgctggaggaagtttcaataatggatttaaccctactactacactaggctgcggatcatggggcagaaacagtatttcagaaaatcttacttacgagcatcttataaatgtttcaagaatagggtatttcaataaagaagcaaaagttcctagctatgaggaaatatggggataa

SEQ ID NO: 18 - protein, 453 aa, Clostridium kluyveri
MSNEVSIKELIEKAKVAQKKLEAYSQEQVDVLVKALGKVVYDNAEMFAKEAVEETEMGVYEDKVAKCHLKSGAIWNHIKDKKTVGIIKEEPERALVYVAKPKGVVAATTPITNPVVTPMCNAMAAIKGRNTIIVAPHPKAKKVSAHTVELMNAELKKLGAPENIIQIVEAPSREAAKELMESADVVIATGGAGRVKAAYSSGRPAYGVGPGNSQVIVDKGYDYNKAAQDIITGRKYDNGIICSSEQSVIAPAEDYDKVIAAFVENGAFYVEDEETVEKFRSTLFKDGKINSKIIGKSVQIIADLAGVKVPEGTKVIVLKGKGAGEKDVLCKEKMCPVLVALKYDTFEEAVEIAMANYMYEGAGHTAGIHSDNDENIRYAGTVLPISRLVVNQPATTAGGSFNNGFNPTTTLGCGSWGRNSISENLTYEHLINVSRIGYFNKEAKVPSYEEIWG

SEQ ID NO: 19 - DNA, 969 bp, Staphylococcus aureus
  source 1..969: mol_type = genomic DNA
atgtcacaacaagatttacctacattattttatagcgggaagtccaatagtgctgttccaattatatctgaaagtgaattacaaacaattacagctgaaccatggcttgaaatttccaaaaaaggattgcaactagaaggattgaactttgatcggcagggacaactctttttattggatgtattcgaaggcaatattttcaaaatcaatcctgaaacgaaggaaatcaaacgaccttttgtaagtcacaaagcgaatcctgcagcaatcaaaatacataaagatggccgattattcgtttgttatttaggagattttaaatctacaggaggcatttttgcagctacagaaaatggtgacaacttacaagatattattgaagatctttcaacagcatattgtattgatgacatggtatttgattctaaaggtggattttattttacagattttagaggatactctaccaatccactaggaggcgtttattatgtttcgccggactttagaacagtgacgcctatcattcaaaatattagcgtagcaaatggtattgctttaagtacagatgaaaaagtactatgggtaacagaaacaacagccaatcgattacatcgcattgcacttgaagatgatggtgtgacgatacaaccatttggagctaccataccgtactattttacaggtcatgaaggaccagactcatgttgtattgatagtgacgataatttatacgtagcaatgtatggtcaaggtcgagtgttagtttttaataaaaggggttatccaataggacaaatattgataccaggccgagatgaagggcatatgttacgttctactcatccgcaatttatacctggaacaaatcaactcatcatttgttccaatgatatagaaatgggcggaggatctatgctttatacagttaatggctttgcaaaaggtcatcaaagttttcagtttcaa

SEQ ID NO: 20 - protein, 323 aa, Staphylococcus aureus
MSQQDLPTLFYSGKSNSAVPIISESELQTITAEPWLEISKKGLQLEGLNFDRQGQLFLLDVFEGNIFKINPETKEIKRPFVSHKANPAAIKIHKDGRLFVCYLGDFKSTGGIFAATENGDNLQDIIEDLSTAYCIDDMVFDSKGGFYFTDFRGYSTNPLGGVYYVSPDFRTVTPIIQNISVANGIALSTDEKVLWVTETTANRLHRIALEDDGVTIQPFGATIPYYFTGHEGPDSCCIDSDDNLYVAMYGQGRVLVFNKRGYPIGQILIPGRDEGHMLRSTHPQFIPGTNQLIICSNDIEMGGGSMLYTVNGFAKGHQSFQFQ

SEQ ID NO: 21 - DNA, 822 bp, Saccharopolyspora erythraea
  source 1..822: mol_type = genomic DNA
agcgggactcccgcccgggaagcgagcagcgctcttcgcgacggctaccggcaggcgggcgtgtcgggcagggtccggtcctacctcgacctgctggcggggctgtcggacttccgcgagcacttcgacggctccgacgggttctccctcgatctcgtggacatggccgacggtcccggagaggtcacggtgatctgctgcgcgggaacggcggcgatctccggtccgcacgagttcacccggctcgccggggcgctgcgcggaatcgctccggttcgggccgtgccccagcccggctacgaggagggcgaacctctgccgtcgtcgatggcggcggtggcggcggtgcaggccgatgcggtcatcaggacacagggggacaagccgttcgtggtggccggtcactccgcgggggcactgatggcctacgcgctggcgaccgaactgctcgatcgcgggcacccgccacgcggtgtcgtcctgatcgacgtctacccgcccggtcaccaggacgcgatgaacgcctggctggaggagctgaccgccacgctgttcgaccgcgagacggtgcggatggacgacaccaggctcaccgccctgggcgcctacgaccgcctcaccggtcagtggcgaccccgggaaaccgggctgccgacgctgctggtcagcgccggcgagccgatgggtccgtggcccgacgacagctggaagccgacgtggcccttcgagcacgacaccgtcgccgtccccggcgaccacttcacgatggtgcaggaacacgccgacgcgatcgcgcggcacatcgacgcctggctgggcgga

SEQ ID NO: 22 - protein, 274 aa, Saccharopolyspora erythraea
SGTPAREASSALRDGYRQAGVSGRVRSYLDLLAGLSDFREHFDGSDGFSLDLVDMADGPGEVTVICCAGTAAISGPHEFTRLAGALRGIAPVRAVPQPGYEEGEPLPSSMAAVAAVQADAVIRTQGDKPFVVAGHSAGALMAYALATELLDRGHPPRGVVLIDVYPPGHQDAMNAWLEELTATLFDRETVRMDDTRLTALGAYDRLTGQWRPRETGLPTLLVSAGEPMGPWPDDSWKPTWPFEHDTVAVPGDHFTMVQEHADAIARHIDAWLGG

SEQ ID NO: 23 - DNA, 831 bp, Streptomyces amphibiosporus
  source 1..831: mol_type = genomic DNA
cccgtattttgggttcatcacggaaatgggggcgttgagagctacgcagcggttgcggaatgctgtggtcgtccgttctatggtattcagccgcgtggctggaccggctccgaagatatcctgaccggtcaagaggcaatggctgcgtactacgtggacatcattcgtgcggtgcagcctgagggtccgtatgatgtgggtggcttcagcctgggtggcctgttcgcctacgaggtggtgcgccagctgcaattgcaagacgcgaccgttgatacgctggttatgttggacactctggacgccgcaagcaccaatttggcgaacagcttaatgaccggtggccgtcaggacgacgcggatgtcgtggccaaagtttccgcgtttcgtgcggtgaacctgatgctaggaaacgattctctcgacgcgcgcgagggcaccagctctgttctgcaccgtgatgaagtcgacaccgcactcgacccggacgctttcctggactcgctggtggaagcggcggttgctcgcggcatccacaagaccccggcgcaattgagaacgcgtgtgcgtcaacttgcccgctatttcgatgctgtgcacggcgaacgtcatgttgtccatccgttgccacgtcgcgaggaagtccgctgctattacctgcgtaatgcaggtggccagtttttcggtccgtttgaagagtatatggttctgtttccggagccggatctgccggcggtcgatggtaccccgtactggcgtgagtgggcagatgccgttgacgattttttcgtgattgacgtagacactgagacgcatgcgcaggttatgaccgaa

SEQ ID NO: 24 - protein, 277 aa, Streptomyces amphibiosporus
PVFWVHHGNGGVESYAAVAECCGRPFYGIQPRGWTGSEDILTGQEAMAAYYVDIIRAVQPEGPYDVGGFSLGGLFAYEVVRQLQLQDATVDTLVMLDTLDAASTNLANSLMTGGRQDDADVVAKVSAFRAVNLMLGNDSLDAREGTSSVLHRDEVDTALDPDAFLDSLVEAAVARGIHKTPAQLRTRVRQLARYFDAVHGERHVVHPLPRREEVRCYYLRNAGGQFFGPFEEYMVLFPEPDLPAVDGTPYWREWADAVDDFFVIDVDTETHAQVMTE

SEQ ID NO: 25 - DNA, 651 bp, Streptomyces sp.
  source 1..651: mol_type = genomic DNA
gtttgtgtatgtccccctttagcccttaatggccctcaaacttataatcgctttgccgcggagctcgcggcggattggaaagttagcgctcttgttgcgccaggttttgccgccgatgctccattacccgaaaattcacgtgtcttaattgccaccttagcggatgctgttcaagaatatgtaggggattcggcttttgcactcgcgggactctcatcaggtggagtcctggcacatgaaattgctcgtgagctcgaacgtcgcggtagtgatcctttagccgtcgtcctgttagatagttacccaatggattcgcctgttttgaaacgctggaagaatgaaatggcgcgtgtgggattagaacgtgcgcgtgcgcttgatcgtcgtcaagcggatgaaggttttcgcttggaagaaattactgcgagcgcatggatttgtggtgagcttcttctggattggcgccctgaagggttgacagctccaagctgtttagtgcgtgcaagtgaaccagttgttgctgaagatggtgcgggatggcaaactcccgtagcagctagtgaagttattgatgtacctggagatcatttctcaattatggatcgccaatttgccgcatcgacggccgcggcagtccgtgcctggctcgcc

SEQ ID NO: 26 - protein, 217 aa, Streptomyces sp.
VCVCPPLALNGPQTYNRFAAELAADWKVSALVAPGFAADAPLPENSRVLIATLADAVQEYVGDSAFALAGLSSGGVLAHEIARELERRGSDPLAVVLLDSYPMDSPVLKRWKNEMARVGLERARALDRRQADEGFRLEEITASAWICGELLLDWRPEGLTAPSCLVRASEPVVAEDGAGWQTPVAASEVIDVPGDHFSIMDRQFAASTAAAVRAWLA

SEQ ID NO: 27 - DNA, 1419 bp, Microbacterium radiodurans
  source 1..1419: mol_type = genomic DNA
atggcaagcccgacgctggacgccgcgatcggcggcgaggtggcggagaccaccacgacctcgccggagcaggtgcagcgcctcgtctcgcgggcgcgggccgcacaggccgtgttcgagaccttctcccaggagaaggtcgacgcgatcgtgcgcgacttcgccaagtacgtctacgacaacgccgaggcgctggccggcatggcccacagggagaccggcctcggcgtctacgaggacaaggtcgccaaggccaagggcaagtcccgtgtcatctggaacaacctcaagggcaagcgctcccgcggcatcatcggcgaggacgcggaggaggggctcatcctggtggccaaacccatgggcgtggtcggggcggtctgcccggtgacgaacccgatcgtcacgccgatgtgcaacgcgatgttcgccctgaaggccggcaacgccattatcttcgcgccacacccgaaggccgacgattccacctggcacctgaccagggagtaccagcggatcgtgcgcgagcacggcggacccgagcatctcgtccaggcggtgcggcgcggctcggtccagaccacccagaccctgatgcaggcggtggacgtggtggtggccaccggcggcggcgccatggtcaagtcggcctactcctcgggcaagccctcctatggggtcggggccggcaacgtgccggtggtgatcgaccgcggcgtcgacctgcgggaggcggcggagaagatcgtggcgggcgcctcgttcgacaacggcatcatctgctcgcacgagcagttcgtgctgacccccgaggagcgctacgccgacacggtcgcggccttccgcgacaccgggcgggtcttcttcaccgacgacccggccgaggtcgaccgggtgcgcgccgtggtgttcgaccacggccatctcaacaaggacgtggtcgggcgctcggcccaggagatcggcgcgcgggccggccttgccgtgcccgagggcgcgcggatcatcctcgtcccggcggacggggcgggcaacgacgacgtgctggccaaggagaagctctgccccgtggtcgcgatcctgccctacggaagcttcgaggaggcggtggaactcgccaaggccaacctcctggtcgagggcgccgggcactcggcggccctgcactcggcggacgaggcgcatatccgcgcgatgggcgtggcgttgccggtgagccggctggtggtcaaccagtcctcggcgctgaccgcgggcggctcgctgacgaacggcttcgcgccgaccacgacgctgggctgcggctcctggggcggcaactcgatctcggaaaacctcgactacaagcacctgatgaacgtctcccgcatcggcaacgtcatcagccgaccggtgccgagcgacgaggagatctgggcggcc

SEQ ID NO: 28 - protein, 473 aa, Microbacterium radiodurans
MASPTLDAAIGGEVAETTTTSPEQVQRLVSRARAAQAVFETFSQEKVDAIVRDFAKYVYDNAEALAGMAHRETGLGVYEDKVAKAKGKSRVIWNNLKGKRSRGIIGEDAEEGLILVAKPMGVVGAVCPVTNPIVTPMCNAMFALKAGNAIIFAPHPKADDSTWHLTREYQRIVREHGGPEHLVQAVRRGSVQTTQTLMQAVDVVVATGGGAMVKSAYSSGKPSYGVGAGNVPVVIDRGVDLREAAEKIVAGASFDNGIICSHEQFVLTPEERYADTVAAFRDTGRVFFTDDPAEVDRVRAVVFDHGHLNKDVVGRSAQEIGARAGLAVPEGARIILVPADGAGNDDVLAKEKLCPVVAILPYGSFEEAVELAKANLLVEGAGHSAALHSADEAHIRAMGVALPVSRLVVNQSSALTAGGSLTNGFAPTTTLGCGSWGGNSISENLDYKHLMNVSRIGNVISRPVPSDEEIWAA

SEQ ID NO: 29 - DNA, 912 bp, Escherichia coli K12
  source 1..912: mol_type = genomic DNA
atggcagatctcaattgggcagcaatcgcgtttatcggtttaggacaaatgggttcgccaatggcgagcaatttattgcagcaagggcatcaacttcgcgtctttgatgtgaacgccgaagctgtgcggcatctggtagacaaaggtgcgactcccgccgccaacccggcgcaggccgctaaagatgccgaatttatcattaccatgttgccgaatggcgatctggtgcgcaacgtgttgttcggtgaaaacggcgtttgcgaaggcttatctaccgatgcgctggtcattgatatgtccaccatccatccgctgcaaaccgataaattgattgccgatatgcaagccaaaggcttcagcatgatggatgttccggtaggccgtacttctgcaaatgccattaccggtactctgttactgctggctggcggcaccgctgaacaagttgaacgtgccacaccgatcctgatggcgatgggcagtgagttgatcaacgctggcggtccgggcatggggatccgcgttaagctcatcaacaactacatgagcatcgcgctcaatgcgctttcggcagaagccgccgttttgtgcgaagccctgaatcttcccttcgatgttgccgtcaaagtgatgagcggtaccgccgccggtaaaggtcacttcaccacttcctggccaaacaaagtcctcagcggagatctttctcccgccttcatgatcgatcttgcccataaggatcttggcatcgcccttgatgtcgccaaccagctgcatgtgccaatgccgctgggggccgcctcacgggaggtttatagccaggcgcgcgcagcgggtcgcggtcgccaggactggtccgccattctggaacaggtccgtgtcagtgccgggatgactgccaaagtaaaaatgtaa

SEQ ID NO: 30 - protein, 303 aa, Escherichia coli K12
MADLNWAAIAFIGLGQMGSPMASNLLQQGHQLRVFDVNAEAVRHLVDKGATPAANPAQAAKDAEFIITMLPNGDLVRNVLFGENGVCEGLSTDALVIDMSTIHPLQTDKLIADMQAKGFSMMDVPVGRTSANAITGTLLLLAGGTAEQVERATPILMAMGSELINAGGPGMGIRVKLINNYMSIALNALSAEAAVLCEALNLPFDVAVKVMSGTAAGKGHFTTSWPNKVLSGDLSPAFMIDLAHKDLGIALDVANQLHVPMPLGAASREVYSQARAAGRGRQDWSAILEQVRVSAGMTAKVKM

SEQ ID NO: 31 - DNA, 1923 bp, Saccharomyces cerevisiae
  source 1..1923: mol_type = genomic DNA
agcgtgtgaggactactcattggcttgcgatttacggtttttatattttttgccgcacatcattttttggcctggtattgtcatcgcgttgagcggactctgaatataatcctattgttttttatggatctctggaagcgtctttttgaagccaacccaacaaaaattcgagacaagaaaataaaaaacggcacttcatcagtatcacaaataccatcaatttatcagctctcatgaatgaaatcgatgagaaaaatcaggcccccgtgcaacaagaatgcctgaaagagatgattcagaatgggcatgctcggcgtatgggatctgttgaagatctgtatgttgctctcaacagacaaaacttatatcggaacttctgcacatatggagaattgagtgattactgtactagggatcagctcacattagctttgagggaaatctgcctgaaaaatccaactcttttacatattgttctaccaataagatggccaaatcatgaaaattattatcgcagttccgaatactattcacggccacatccagtgcatgattatatttcagtattacaggaattgaaactgagtggtgtggttctcaatgaacaacctgagtacagtgcagtaatgaagcaaatattagaagaattcaaaaatagtaagggttcctatactgcaaaaatttttaaacttactaccactttgactattccttactttggaccaacaggaccgagttggcggctaatttgtcttccagaagagcacacagaaaagtggaaaaaatttatctttgtatctaatcattgcatgtctgatggtcggtcttcgatccacttttttcatgatttaagagacgaattaaataatattaaaactccaccaaaaaaattagattacattttcaagtacgaggaggattaccaattgttgaggaaacttccagaaccgatcgaaaaggtgatagactttagaccaccgtacttgtttattccgaagtcacttctttcgggtttcatctacaatcatttgagattttcttcaaaaggtgtctgtatgagaatggatgatgtggaaaaaaccgatgatgttgtcaccgagatcatcaatatttcaccaacagaatttcaagcgattaaagcaaatattaaatcaaatatccaaggtaagtgtactatcactccgtttttacatgtttgttggtttgtatctcttcataaatggggtaaatttttcaaaccattgaacttcgaatggcttacggatatttttatccccgcagattgccgctcacaactaccagatgatgatgaaatgagacagatgtacagatatggcgctaacgttggatttattgacttcaccccatggataagcgaatttgacatgaatgataacaaagaaaaattttggccacttattgagcactaccatgaagtaatttcggaagctttaagaaataaaaagcacctccatggcttagggttcaatatacaaggcttcgttcaaaaatatgtgaatattgacaaggtaatgtgcgatcgtgccatcgggaaaagacgcggaggtacattgttaagcaatgtaggtctgtttaatcagttagaggagcccgatgccaaatattctatatgcgatttggcatttggccaatttcaaggatcctggcaccaagcattttccttgggtgtttgttcgactaatgtaaaggggatgaatattgttgttgcttcaacaaaaaatgttgttggtagccaagaatctctcgaagagctttgctccatttataaagctctccttttaggcccttagatctcacatgatgcttgactgatattattcgacaatatgattatgtcgtgtaaataacccactttcatgttgtcactccctcggctttggttggttaaagggacttattggt

SEQ ID NO: 32 - protein, 525 aa, Saccharomyces cerevisiae
MNEIDEKNQAPVQQECLKEMIQNGHARRMGSVEDLYVALNRQNLYRNFCTYGELSDYCTRDQLTLALREICLKNPTLLHIVLPTRWPNHENYYRSSEYYSRPHPVHDYISVLQELKLSGVVLNEQPEYSAVMKQILEEFKNSKGSYTAKIFKLTTTLTIPYFGPTGPSWRLICLPEEHTEKWKKFIFVSNHCMSDGRSSIHFFHDLRDELNNIKTPPKKLDYIFKYEEDYQLLRKLPEPIEKVIDFRPPYLFIPKSLLSGFIYNHLRFSSKGVCMRMDDVEKTDDVVTEIINISPTEFQAIKANIKSNIQGKCTITPFLHVCWFVSLHKWGKFFKPLNFEWLTDIFIPADCRSQLPDDDEMRQMYRYGANVGFIDFTPWISEFDMNDNKENFWPLIEHYHEVISEALRNKKHLHGLGFNIQGFVQKYVNIDKVMCDRAIGKRRGGTLLSNVGLFNQLEEPDAKYSICDLAFGQFQGSWHQAFSLGVCSTNVKGMNIVVASTKNVVGSQESLEELCSIYKALLLGP

SEQ ID NO: 33 - DNA, 2623 bp, Saccharomyces cerevisiae
  source 1..2623: mol_type = genomic DNA
gacccttttctcgatatgtttttctgtgatttttttttttcaattgttcgaaataacctgtattgttcaatactggacaattgttctgatatatatatatatatataattttcaatgaaaaaaaaaaaaaaaagaaagaaaattagaagctagaaagaaataaggaaaaggaaaagcaaacaacaataggaaaaaaaagcatagaaacatataatagattaaatacaaaagatttattatacatgcgaggtaagactattgtctaagacttatacccgaccgagagcagccacggcatgcatcgactacgagcgtattgtgttatatcagctctgtccgatacactgcttgccttttgtacgagctcggccgagctatacgaaggcccgctacggcagtatcgcattaaagcgacgcaaattcgccgatggtttggtagaagagcttgcagaacgattcccattcgccacgatcccgtagagtgccctgaaccacgctcatgtccatgttcataccattcacgtttgtggagcaaacgtttaggccgaacgcaaatctcaaggcgcctgcagactgcgagaagattaaatcccgaacgtagtacggcttggtcgtatcttgcgagacaagtcctgtattactgatgattgcaccgcctctttgctggtgcaaataagagttggcaagaaggctgtctatattcttgttttcaacgataaaatcaagttgtaggacccccagaccaatcaagtggtcaccgttgtcgtacgattctaaaaagcggtcatagtagtattcgacaagactccaaaacttgtcattgtcaccttcgggaatgtcgaatgaggagattagataggcgtaatgcgaacctccaacgttggagccgtatttataagaatctcttaactcttcatcgtttggtaaaaaccttcttgcgttgcttggaatagccacatcgaacccatactcgagaagagattttgtgaacagcttatccagtctatacagggctacaagaaagcacgcttgaatgaagggtgttagggtgcacccatcgtgaacatttttcttgatctgagctctgatttgttctacttgcttagtactgaaatgcaacaagtaattgtaactagcattagcattactagaggggctatatctagctgtaagtgtagattcgctggaggttttaaaattacaatgttcataaatgaaggttgttaaaaagaatttgggtaatgaagtcaatgctggcttgtagtcaatacgatccgtaatcggttttggcaatttagaaatttcagtgtagtctcgatcatagtcaatgatgacttgatcttcgatgaactcttcatcataatcaaaccctttttcttcgattttacaaaagagtagagctaaatctttgaaaaaattcgatccactgacaccgtcggagccgcagtggttagtgacataaacgaagtttttccacgtttcaaacccatcagtatccttacctggtaaacaaatcaatctccaattaggcctcttcggattacccagaggtatgattacagggctaatcaattcgattaacctattggtgactttgaaatcatcctttttgaactgttctgagattttctccatgacttctctgtattctggttgattattcataatcaagtcatcgaattcaagatgagaaatcacctttatgaaatcatgctgtggaaaaggtttactaaggtactcttcgctagagtagtacgcttcatgatgaggataatgcttaggaatgattgtatgcgcaagagttgagtatttttgaagtaatactttcaagacaagcattagttgtctcttattaacacctttattcaattccgcgtaaacagtaaaattcgagtacattttctgcctactcaaaacagcaaagtagttttccaagtggcccatacgtcttgcatggccacggtctatcaactcttgagtgatatgtggttcgtatccttctatatcttccataattaacctggacaatttttattgctgaagttattattcgtttgaaagtctcagtactgtttgtacgcattaggaatgacattgctgttactgcagaattgccctacacatttgttgttgtcttttaagtaacaatttgctctattttcacagacctcgaagaaacgtaaatcttgacaacaaatgtttacgatcgtttaggtgtatgaaaaaatatagaataccaaaataggaaactcgttgaattcgtttactcattatcccgcaatatttatcggtggcctacagagttcaatgtagcctgagattgtggcgattgaaatactgattacttaaaatttgctaaagcctgttctatctgaagaaaaaaaaaaaaaatggatgagccttccagttcattgaaattcttcagttcttatttttttttcaaaaagaaaagtttcagtaccctgttccaaaaaaaatgaaaaaaatgaaaatccagtatccttcccataaaaattaaaagttccattatttatgggtaagtaagtaccactgattatataccatatttataaagtgcatgaatttattactatatattgctttttctgacgtgcttccttca

SEQ ID NO: 34 - protein, 535 aa, Saccharomyces cerevisiae
MEDIEGYEPHITQELIDRGHARRMGHLENYFAVLSRQKMYSNFTVYAELNKGVNKRQLMLVLKVLLQKYSTLAHTIIPKHYPHHEAYYSSEEYLSKPFPQHDFIKVISHLEFDDLIMNNQPEYREVMEKISEQFKKDDFKVTNRLIELISPVIIPLGNPKRPNWRLICLPGKDTDGFETWKNFVYVTNHCGSDGVSGSNFFKDLALLFCKIEEKGFDYDEEFIEDQVIIDYDRDYTEISKLPKPITDRIDYKPALTSLPKFFLTTFIYEHCNFKTSSESTLTARYSPSSNANASYNYLLHFSTKQVEQIRAQIKKNVHDGCTLTPFIQACFLVALYRLDKLFTKSLLEYGFDVAIPSNARRFLPNDEELRDSYKYGSNVGGSHYAYLISSFDIPEGDNDKFWSLVEYYYDRFLESYDNGDHLIGLGVLQLDFIVENKNIDSLLANSYLHQQRGGAIISNTGLVSQDTTKPYYVRDLIFSQSAGALRFAFGLNVCSTNVNGMNMDMSVVQGTLRDRGEWESFCKLFYQTIGEFASL

SEQ ID NO: 35 - DNA, 1397 bp, Saccharomyces cerevisiae
  source 1..1397: mol_type = genomic DNA
tagcaatgtctgcagagactttctccgcttgcataaccttataaacttataacgatacctacttatttcatatcttatgtatagttctccttcaaggagacgttgttttaatacttttatctattccatttatcattatctactttgtaatcaaagatcgaagcactgctgtgtgcgttgatttgggcctgacacgagaagaaagtgcgttacgtacatcaagattttattcaaaatccagcgtctcctccgcaaaaaagttcagaatacatttgaatgagtcctctggattctccaacagtaaattatgcccagtattaaattctcggacatcagcacagggaaagttatacctcaggagggaataatcattgttaataaagttagactgcaggcctttcatgaataaaactttacgactggctgtgcctttttggaagaagtccctttggttcgatagatccggccattttttcacttcatccaacaggttcggcatgctggagagaggcattgagtaatttatataaggatcatgctgttgttgttcatagtgtaactgtgcttggtgtacattattggattttacagaaagaaaaccgttggcaaagtagagtgccacagctccaccacatttcctcttattacattccaaacttttgaaaagctcaagaacttttttctgccagctgggggaacctcttaaaatcttgacatctctgcggataatccgcattattaagtcatagttttgtaatatcatgggatctaattctggggtttcataaggcggtaaatcaattgatatacactttcgtatattaatattcttgtacaatgtggttaataatgcaattttaccacccatagagaatcccagtaaataaataggtctctccaagccaatatgagtttcaataaaataaatcaaatcattggtcagtgtagtgtagtcataaggtattgcttttggtgaaatgccatgatttcttacatcaacagaaaaaatatccgcgtcaagcttccttgaaagaagcttattgagagaatgaaacatcacgtgtgagcccaaaaggccatggatgttaataattgcaggtctttgctcgaattttattagcgatgaaacatctgagggtaaacgcgttctatggaatgataaatcgacaattattttataaggtagcgctttattatgcgctagtctcgacatttgtaagccctcttcccacggttccttttattttgcagtttgtatacgtctcgaccttggcatgagacaaaaacagtttccccaaaagacgcgctgtggtattcagtaaaggctggtttctatgataggttgagtttagaataatttttaaacaggatactaagataaagcaaatatttcaatc

SEQ ID NO: 36 - protein, 328 aa, Saccharomyces cerevisiae
MSRLAHNKALPYKIIVDLSFHRTRLPSDVSSLIKFEQRPAIINIHGLLGSHVMFHSLNKLLSRKLDADIFSVDVRNHGISPKAIPYDYTTLTNDLIYFIETHIGLERPIYLLGFSMGGKIALLTTLYKNINIRKCISIDLPPYETPELDPMILQNYDLIMRIIRRDVKILRGSPSWQKKVLELFKSLECNKRKCGGAVALYFANGFLSVKSNNVHQAQLHYEQQQHDPYINYSMPLSSMPNLLDEVKKWPDLSNQRDFFQKGTASRKVLFMKGLQSNFINNDYSLLRYNFPCADVREFNTGHNLLLENPEDSFKCILNFFAEETLDFE

SEQ ID NO: 37 - DNA, 648 bp, Mycobacterium smegmatis
  source 1..648: mol_type = genomic DNA
atggccaagcgaattctgtgtttcggtgattccctgacctggggctgggtccccgtcgaagacggggcacccaccgagcggttcgcccccgacgtgcgctggaccggtgtgctggcccagcagctcggagcggacttcgaggtgatcgaggagggactgagcgcgcgcaccaccaacatcgacgaccccaccgatccgcggctcaacggcgcgagctacctgccgtcgtgcctcgcgacgcacctgccgctcgacctggtgatcatcatgctgggcaccaacgacaccaaggcctacttccggcgcaccccgctcgacatcgcgctgggcatgtcggtgctcgtcacgcaggtgctcaccagcgcgggcggcgtcggcaccacgtacccggcacccaaggtgctggtggtctcgccgccaccgctggcgcccatgccgcacccctggttccagttgatcttcgagggcggcgagcagaagaccactgagctcgcccgcgtgtacagcgcgctcgcgtcgttcatgaaggtgccgttcttcgacgcgggttcggtgatcagcaccgacggcgtcgacggaatccacttcaccgaggccaacaatcgcgatctcggggtggccctcgcggaacaggtgcggagcctgctg

SEQ ID NO: 38 - protein, 216 aa, Mycobacterium smegmatis
MAKRILCFGDSLTWGWVPVEDGAPTERFAPDVRWTGVLAQQLGADFEVIEEGLSARTTNIDDPTDPRLNGASYLPSCLATHLPLDLVIIMLGTNDTKAYFRRTPLDIALGMSVLVTQVLTSAGGVGTTYPAPKVLVVSPPPLAPMPHPWFQLIFEGGEQKTTELARVYSALASFMKVPFFDAGSVISTDGVDGIHFTEANNRDLGVALAEQVRSLL

SEQ ID NO: 39 - DNA, 1080 bp, Homo sapiens
  source 1..1080: mol_type = genomic DNA
atgctgagtgccgcgtctcgcgtagtctcccgcgccgccgtccactgcgcgcttcgctctccgccgcccgaggcccgcgcgctcgccatgtcccggccaccgccaccgcgggtcgcctcggtgctgggcaccatggagatggggcgccgcatggacgcgcccgccagcgccgcggccgtgcgcgcctttctggagcgcggccacaccgaactggacacggccttcatgtacagcgacggccagtccgagaccatcctgggcggcctggggctcgggctgggcggtggcgactgcagagtgaaaattgccaccaaggccaacccttgggatggaaaatcactaaagcctgacagtgtccggtcccagctggagacgtcattgaagaggctgcagtgtccccaagtggacctcttctacctacacgcacctgaccacggcaccccggtggaagagacgctgcatgcctgccagcggctgcaccaggagggcaagttcgtggagcttggcctctccaactatgctagctgggaagtggccgagatctgtaccctctgcaagagcaatggctggatcctgcccactgtgtaccagggcatgtacaacgccaccacccggcaggtggaaacggagctcttcccctgcctcaggcactttggactgaggttctatgcctacaaccctctggctgggggcctgctgactggcaagtacaagtatgaggacaaggacgggaaacagcctgtgggccgcttctttgggaatagctgggctgagacctacaggaatcgcttctggaaggagcaccacttcgaggccattgcgttggtggagaaggccctgcaggccgcatatggcgccagcgcccccagtgtgacctcggctgccctccggtggatgtaccaccactcacagctgcagggtgcccacggggacgcggtcatcctgggcatgtccagcctggagcagctggagcagaacttggcagcaacagaggaagggcccctggagccggctgtcgtggatgcctttaatcaagcctggcatttggttgctcacgaatgtcccaactacttccgctag

SEQ ID NO: 40 - protein, 359 aa, Homo sapiens
MLSAASRVVSRAAVHCALRSPPPEARALAMSRPPPPRVASVLGTMEMGRRMDAPASAAAVRAFLERGHTELDTAFMYSDGQSETILGGLGLGLGGGDCRVKIATKANPWDGKSLKPDSVRSQLETSLKRLQCPQVDLFYLHAPDHGTPVEETLHACQRLHQEGKFVELGLSNYASWEVAEICTLCKSNGWILPTVYQGMYNATTRQVETELFPCLRHFGLRFYAYNPLAGGLLTGKYKYEDKDGKQPVGRFFGNSWAETYRNRFWKEHHFEAIALVEKALQAAYGASAPSVTSAALRWMYHHSQLQGAHGDAVILGMSSLEQLEQNLAATEEGPLEPAVVDAFNQAWHLVAHECPNYFR

SEQ ID NO: 41 - DNA, 2772 bp, Saccharomyces cerevisiae
  source 1..2772: mol_type = genomic DNA
atgtcaagcggcagtactattgtttcgtctgacaagtcggggcgaacatttaagcatgaggaagaactacccaagttaccattacctaaactctgtgacactttgcagcgtctgaaggagagtttagagccattgtattatgctgatggttactatcaacatcctttagatccagaacaaattgagaagctgtcatccataataagagattttgaagagaatcccgtaagtgagaagctgcaatctaaactacagagctaccatgacactagggattgctaccttgatgaattacacttagacatcaataaccagacctccactagggagattcaggacgatgtcctacccaggaatcccttcttggttctcgcagatgacgctttgccaaacatcactcaagctgataggtcagccgtcctagtgcactctgcggccaggtttatttctgctttgaaacaagatttgttgccaccagatatcaatgccaccaatggcaaaccactgtcaatggctccatttctgaacttgttcggtactactcgtagtcctgtttttcagcgcggtgaggttgaaaattttgatttgaacaaaccttacacagcatctgatttagaagaccctgattattcaagcgatgaggacgataacgacgaacccacccaaaaagactttgatgaccgtaaaagaaaacacgaagaagacatattcactggcaatggtataactataaaaagacatccggatagcaaacacatcttgatcatctccaggggccaatactatacactggaagtacttgattcgacgaataaaattatatacactgctgcagaattaaccacaattttcaaccatataataaaagattcttctggcattgaaaaatcaactgcgttaggtagtttgacctctcactcttttagaaactggaaatatgcaaggaagagactacagaaaaggtatcctaatgaattacatcgcatagactctgctctgttcgtgttagtgctggacgaatcacaagaagaaactaccaatgatggcgatgataccgctgatatcagtcaaatgtttaacaggactatcactgagcgagacaagaaatgtacttccgccaattgtaagagagtgttttacggtacttctataataaatagcaaaggccatcaagtgggttcatgtgtttctcgttggtatgataaattacagttagttgtcactgcagatgcaaaagccacagtgatttgggattccttcacatgtgacgggtcagtcgtgcttcggtttacttctgaaatttatacagaatcagtcttgagattagctagagacgttaacgccggtgacccacagttttcactctggcctaacgtgacacaaatggatcctgaaactaaaaaactgatgacggcgaccataagtgccgacggcggaggcccttctgaaattgaccctaagttggttgtaaataaaatcgattggtctttcagtaacatcctgaacacacatgtccatttgtcagaaaccaaactagctgatctgatttccaagtatgatattgttcgcgcttctattccattgggcagaaggtcagctcaaagattaggagtcaagcccgattcgatggttcaagtagctttacaaatagctcactatgctttgtatggaagaatggtgtttggattagaaccagtatccacacgtggtttcaaaaactcgagatcatcttttattaacattcaaagtcaagcgctgttggaactatgtcaattgtttatttctagctccattgatgggacagataaattagacaaatttatccaaacctgcgaaacacataataacatggttaaacatgcaaagtctggggtaggttatgaaaaacacttcaatgcattgaaatatctcttcaaatttcatgaccattttggtatccacttaagtggtgatgaatcatctgctgcgaaagatcttttcgaaaatccacttgttttacctttctcacagcctgagttgattgttgccaactgtggtaatgctgctaccacgacatttggtataactcctgctgtacctcatggctttggaatagggtacatcattaaagatgatcaagtagatttgaccgtgacatcgcaatttcgacagggtgacaggttaatgtttatgttaagttgggtgttaggcgaaattcgttcctactggaggatgtcacgcggcacttctcataataaaactggcgtgaagatcagtccagtggtcgataaattgtatgaaatggacaatgcagtcaataatcctccgaaacgtaacggccatacagtcaatggctcccgcaaaacatcttcttcatcacaggtaaatttgaacaggtatggcgggtttttcgatttggaaggccacattgacagtaggaatatatctaaaacaccatcgatgaagaatttgcagaaaaccttcaatggcttgaccatgagcgcagacaatgatcattcaagttccgcagtttccgttcccacagaaaaggaaaaattaaatacaggccatgaaattttacagatccaaccgcgggaagtggcaagtaatgggttggaagcagatgatgagacagatattgaaatagttgctggcaatgctgatggcacatcctcttcggcatcttcagctacatctctaaattccaagaaacgtaacgtcatcaactcaagattcgatattgattttgaccgcagtcgtgtgggtagaaaggtggcgaccttagatcaataa

SEQ ID NO: 42 - protein, 923 aa, Saccharomyces cerevisiae
MSSGSTIVSSDKSGRTFKHEEELPKLPLPKLCDTLQRLKESLEPLYYADGYYQHPLDPEQIEKLSSIIRDFEENPVSEKLQSKLQSYHDTRDCYLDELHLDINNQTSTREIQDDVLPRNPFLVLADDALPNITQADRSAVLVHSAARFISALKQDLLPPDINATNGKPLSMAPFLNLFGTTRSPVFQRGEVENFDLNKPYTASDLEDPDYSSDEDDNDEPTQKDFDDRKRKHEEDIFTGNGITIKRHPDSKHILIISRGQYYTLEVLDSTNKIIYTAAELTTIFNHIIKDSSGIEKSTALGSLTSHSFRNWKYARKRLQKRYPNELHRIDSALFVLVLDESQEETTNDGDDTADISQMFNRTITERDKKCTSANCKRVFYGTSIINSKGHQVGSCVSRWYDKLQLVVTADAKATVIWDSFTCDGSVVLRFTSEIYTESVLRLARDVNAGDPQFSLWPNVTQMDPETKKLMTATISADGGGPSEIDPKLVVNKIDWSFSNILNTHVHLSETKLADLISKYDIVRASIPLGRRSAQRLGVKPDSMVQVALQIAHYALYGRMVFGLEPVSTRGFKNSRSSFINIQSQALLELCQLFISSSIDGTDKLDKFIQTCETHNNMVKHAKSGVGYEKHFNALKYLFKFHDHFGIHLSGDESSAAKDLFENPLVLPFSQPELIVANCGNAATTTFGITPAVPHGFGIGYIIKDDQVDLTVTSQFRQGDRLMFMLSWVLGEIRSYWRMSRGTSHNKTGVKISPVVDKLYEMDNAVNNPPKRNGHTVNGSRKTSSSSQVNLNRYGGFFDLEGHIDSRNISKTPSMKNLQKTFNGLTMSADNDHSSSAVSVPTEKEKLNTGHEILQIQPREVASNGLEADDETDIEIVAGNADGTSSSASSATSLNSKKRNVINSRFDIDFDRSRVGRKVATLDQ

SEQ ID NO: 43 - DNA, 612 bp, Escherichia coli K12
  source 1..612: mol_type = genomic DNA
ttgaacatgccaatgaccgaaagaataagagcaggcaagctatttaccgatatgtgcgaaggcttaccggaaaaaagacttcgtgggaaaacgttaatgtatgagtttaatcactcgcatccatcagaagttgaaaaaagagaaagcctgattaaagaaatgtttgccacggtaggggaaaacgcctgggtagaaccgcctgtctatttctcttacggttccaacatccatataggccgcaatttttatgcaaatttcaatttaaccattgtcgatgactacacggtaacaatcggtgataacgtactgattgcacccaacgttactctttccgttacgggacaccctgtacaccatgaattgagaaaaaacggcgagatgtactcttttccgataacgattggcaataacgtctggatcggaagtcatgtggttattaatccaggcgtcaccatcggggataattctgttattggcgcgggtagtatcgtcacaaaagacattccaccaaacgtcgtggcggctggcgttccttgtcgggttattcgcgaaataaacgaccgggataagcactattatttcaaagattataaagttgaatcgtcagtttaa

SEQ ID NO: 44 - protein, 203 aa, Escherichia coli K12
MNMPMTERIRAGKLFTDMCEGLPEKRLRGKTLMYEFNHSHPSEVEKRESLIKEMFATVGENAWVEPPVYFSYGSNIHIGRNFYANFNLTIVDDYTVTIGDNVLIAPNVTLSVTGHPVHHELRKNGEMYSFPITIGNNVWIGSHVVINPGVTIGDNSVIGAGSIVTKDIPPNVVAAGVPCRVIREINDRDKHYYFKDYKVESSV

SEQ ID NO: 45 - DNA, 1473 bp, Aspergillus terreus
  source 1..1473: mol_type = genomic DNA
atgaccaaacagagcgcagatagcaatgcaaaaagcggtgttaccagcgaaatttgtcattgggcaagcaatctggcaaccgatgatattccgagtgatgttctggaacgtgccaaatatctgattctggatggtattgcatgtgcatgggttggtgcacgtgttccgtggtcagaaaaatatgttcaggcaaccatgagctttgaaccgcctggtgcatgtcgtgttattggttatggccagaaactgggtccggttgcagcagcaatgaccaatagcgcatttattcaggccaccgaactggatgattatcatagcgaagcaccgctgcatagcgcaagcattgttctgcctgcagtttttgcagcaagcgaagttctggcagaacagggtaaaaccattagcggtattgatgttattctggcagccattgttggttttgaaagcggtccgcgtattggtaaagcaatttatggtagcgatctgctgaataatggttggcattgtggtgcagtttatggtgcaccggcaggcgcactggccaccggtaaactgctgggtctgacaccggatagcatggaagatgcactgggtattgcctgtacccaggcatgtggtctgatgagcgcacagtatggtggtatggttaaacgtgttcagcatggttttgcagcccgtaatggtctgctgggtggcctgctggcacatggtggttatgaagcaatgaaaggtgtgctggaacgtagctatggtggttttctgaaaatgtttaccaaaggcaatggtcgtgaacctccgtataaagaagaagaagttgttgcaggtctgggtagcttttggcatacctttaccattcgcattaaactgtatgcatgttgtggtctggttcatggtccggtggaagcaattgaaaatctgcagggtcgttatccggaactgctgaatcgtgcaaatctgagcaatattcgtcatgttcatgttcagctgagcaccgcaagcaatagccattgcggttggattccggaagaacgtccgattagcagcattgcaggtcagatgagcgttgcatatattctggccgttcagctggttgatcagcagtgtctgctgagccagtttagcgaatttgatgataacctggaacgtccggaagtttgggatctggcacgtaaagttaccagcagccagagcgaagaatttgatcaggatggtaattgtctgagcgcaggtcgtgttcgtattgaatttaatgatggttccagcattaccgaaagcgttgaaaaaccgctgggtgttaaagaaccgatgccgaatgaacgtatcctgcataaatatcgtaccctggcaggtagcgttaccgatgaaagccgtgtgaaagaaattgaagatctggttctgggtctggatcgtctgaccgatattagtccgctgctggaactgctgaactgtccggttaaaagtccgctggtttaa

SEQ ID NO: 46 - protein, 490 aa, Aspergillus terreus
MTKQSADSNAKSGVTSEICHWASNLATDDIPSDVLERAKYLILDGIACAWVGARVPWSEKYVQATMSFEPPGACRVIGYGQKLGPVAAAMTNSAFIQATELDDYHSEAPLHSASIVLPAVFAASEVLAEQGKTISGIDVILAAIVGFESGPRIGKAIYGSDLLNNGWHCGAVYGAPAGALATGKLLGLTPDSMEDALGIACTQACGLMSAQYGGMVKRVQHGFAARNGLLGGLLAHGGYEAMKGVLERSYGGFLKMFTKGNGREPPYKEEEVVAGLGSFWHTFTIRIKLYACCGLVHGPVEAIENLQGRYPELLNRANLSNIRHVHVQLSTASNSHCGWIPEERPISSIAGQMSVAYILAVQLVDQQCLLSQFSEFDDNLERPEVWDLARKVTSSQSEEFDQDGNCLSAGRVRIEFNDGSSITESVEKPLGVKEPMPNERILHKYRTLAGSVTDESRVKEIEDLVLGLDRLTDISPLLELLNCPVKSPLV

SEQ ID NO: 47 - DNA, 1314 bp, Corynebacterium glutamicum
  source 1..1314: mol_type = genomic DNA
atgtttgaacgcgatattgttgccaccgataataacaaagcagttctgcattatccgggtggtgaatttgaaatggatatcattgaagccagcgaaggtaataatggtgttgttctgggtaaaatgctgagcgaaaccggtctgattacctttgatccgggttatgttagcaccggtagcaccgaaagtaaaatcacctatattgatggtgatgccggtattctgcgttatcgcggttatgatattgcagatctggccgaaaatgccacctttaatgaagttagttacctgctgattaacggtgaactgccgactccggatgaactgcacaaatttaacgatgaaattcgccatcataccctgctggatgaagatttcaaaagtcagtttaacgtgtttccgcgtgatgcacatccgatggcaaccctggccagcagcgttaatattctgagcacctattatcaggatcagctgaatccgctggatgaggcacagctggataaagcaaccgttcgtctgatggcaaaagttccgatgctggcagcatatgcacatcgtgcccgtaaaggtgcaccgtatatgtatccagataatagcctgaatgcccgtgaaaactttctgcgtatgatgtttggttatccgaccgaaccgtatgaaattgatccgattatggtgaaagcactggacaaactgctgattctgcatgccgatcatgaacagaattgtagcaccagcaccgttcgcatgattggtagcgcacaggcaaatatgtttgttagcattgccggtggtattaatgcactgagtggtccgctgcatggtggtgcaaatcaggcagttctggaaatgctggaagatatcaaatcaaatcatggcggtgatgccaccgagtttatgaataaagtgaaaaacaaagaggatggcgtccgcctgatgggttttggtcatcgtgtgtataaaaactatgatccgcgtgccgcaatcgttaaagaaaccgcacatgaaattctggaacatctgggtggtgatgatctgctggatctggcgattaaactggaagaaattgcactggcagatgactattttatcagccgcaaactgtatccgaacgtggatttctatacaggtctgatttatcgtgcaatgggctttccgaccgattttttcaccgttctgtttgcaattggtcgtctgcctggttggattgcacattatcgtgaacagctgggtgcagcaggcaataaaatcaatcgtccgcgtcaggtttataccggtaatgaaagtcgtaaactggttcctcgtgaagaacgttaa

SEQ ID NO: 48 - protein, 437 aa, Corynebacterium glutamicum [sequence omitted]

SEQ ID NO: 49 - DNA, 1374 bp, Rosa hybrid cultivar
  source 1..1374: mol_type = genomic DNA
atggagaaaattgaggtcagtattatttcccgagacaccattaaaccatcagctgcttcctcttcactacacccttacaagctttccatcatcgatcagttcactcccacaacgtatttcccagttatattcttctaccccattactgaccgtgtcttcaatcttcctcaaaccttaaccgacttgaaaaacactgtttcccaggctctcactttgtaccatccactctccgggaggataaaaaacaacctatacattgatgatttcgaagcaggcatcccctaccttgaggcccgagtgaattttcacatgattgattttctaaggcttccgaaaatcgagtggctaaatgagtttgttccaatggctccatatcgcaaggaaacaatatctgagtttcttcccttgcttggaattcaagtaaacattttcgactctggaatagcaattggtgtctctttctctcacaagatcaacgatggccaaacggcaagctgttttctcaagtcctgggttgctatttttcgtgggtatcgtaacaaaatcatacatcctaatctctctcaagctgcattacttttgccatcgagggatgacttgcctgaaaagtacgtagctatgatggaaaggatgtggtttggcgagaaaaaagttgttacaaggagatttgtatttgatgcgaaagccatatccgcacttcaagatgaagggaagagcgaatacgtgcccaagccatcacgtgttcaggccctcactggttttctctggaaacatcaactcgctgcttctcgggcattatcatcaggtacttcaacaagattttccgtagcatcacagacagtgaacttaaggtcaaaaatgaacatgaaaacgacgttggacaatgccattggtaatatctttttgtgggcttcggcacggctagatctaaatgatacagcaccagggagcagtgatcttaagttgtgtgacttggttaacttactcaatgaatctatcaaagaatttaacagtgattacttggagattttgaagggtaaagagggatatggaggcatgtgtgatttgctagatttcatggaagaagggagttttgtagaaccagcaccagagttttattcattctcaagctggactagattttttgaccaagttgattttggatgggggaggccatcttgggttggattctcggggagagttgaaactagaaatttcacaatattcgttgaaacacaatgcgatgacggaattgatgcgtgggtgactgtagatgaaaaacaaatggctatgctagaacaagatccacagtttttagcatttgcatctccaaacccccgaatttcaatagcctcttcagttggtatggattaa

SEQ ID NO: 50 - protein, 457 aa, Rosa hybrid cultivar
MEKIEVSIISRDTIKPSAASSSLHPYKLSIIDQFTPTTYFPVIFFYPITDRVFNLPQTLTDLKNTVSQALTLYHPLSGRIKNNLYIDDFEAGIPYLEARVNFHMIDFLRLPKIEWLNEFVPMAPYRKETISEFLPLLGIQVNIFDSGIAIGVSFSHKINDGQTASCFLKSWVAIFRGYRNKIIHPNLSQAALLLPSRDDLPEKYVAMMERMWFGEKKVVTRRFVFDAKAISALQDEGKSEYVPKPSRVQALTGFLWKHQLAASRALSSGTSTRFSVASQTVNLRSKMNMKTTLDNAIGNIFLWASARLDLNDTAPGSSDLKLCDLVNLLNESIKEFNSDYLEILKGKEGYGGMCDLLDFMEEGSFVEPAPEFYSFSSWTRFFDQVDFGWGRPSWVGFSGRVETRNFTIFVETQCDDGIDAWVTVDEKQMAMLEQDPQFLAFASPNPRISIASSVGMD

SEQ ID NO: 51 - DNA, 8202 bp, synthetic construct
  source 1..8202: mol_type = other DNA
ggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatttgatatcgagctcgcttggactcctgttgatagatccagtaatgacctcagaactccatctggatttgttcagaacgctcggttgccgccgggcgttttttattggtgagaatccaagcctcgagctgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctgtggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcctgcataacgcgaagtaatcttttcggttttaaagaaaaagggcagggtggtgacaccttgcccttttttgccggactgcagggtctctgtaccttccttcaacaatacaacaatagcgtgctgttcgcacgctatcgattacatttttactttggcagtcatcccggcactgacacggacctgttccagaatggcggaccagtcctggcgaccgcgacccgctgcgcgcgcctggctataaacctcccgtgaggcggcccccagcggcattggcacatgcagctggttggcgacatcaagggcgatgccaagatccttatgggcaagatcgatcatgaaggcgggagaaagatctccgctgaggactttgtttggccaggaagtggtgaagtgacctttaccggcggcggtaccgctcatcactttgacggcaacatcgaagggaagattcagggcttcgcacaaaacggcggcttctgccgaaagcgcattgagcgcgatgctcatgtagttgttgatgagcttaacgcggatccccatgcccggaccgccagcgttgatcaactcactgcccatcgccatcaggatcggtgtggcacgttcaacttgttcagcggtgccgccagccagcagtaacagagtaccggtaatggcatttgcagaagtacggcctaccggaacatccatcatgctgaagcctttggcttgcatatcggcaatcaatttatcggtttgcagcggatggatggtggacatatcaatgaccagcgcatcggtagataagccttcgcaaacgccgttttcaccgaacaacacgttgcgcaccagatcgccattcggcaacatggtaatgataaattcggcatctttagcggcctgcgccgggttggcggcgggagtcgcacctttgtctaccagatgccgcacagcttcggcgttcacatcaaagacgcgaagttgatgcccttgctgcaataaattgctcgccattggcgaacccatttgtcctaaaccgataaacgcgattgctgcccaattgagatctgccatatggctgccgcgcggcaccaggccgctgctgtgatgatgatgatgatggctgctgcccatggtatatctccttcttaaagttaaacaaaattatttctagaggggaattgttatccgctcacaattcccctatagtgagtcgtattaatttcgcgggatagccggaacaatacaacaatagcgtgcgtggttagcacgctatcgattatccccatatttcctcatagctaggaacttttgcttctttattgaaataccctattcttgaaacatttataagatgctcgtaagtaagattttctgaaatactgtttctgccccatgatccgcagcctagtgtagtagtagggttaaatccattattgaaacttcctccagcagtagttgcaggctgatttacaactaatctgcttataggtaatacagttcctgcatatcttatgttctcgtcattgtcagaatgtatgcctgctgtatgaccagctccttcatacatataattagccatagctatttcaactgcttcttcaaaagtatcatatttcaatgctactaaaactggacacattttttctttacaaagtacatctttttctcctgcacctttacccttaagtactataaccttagtaccttctggtacttttactcctgcaagatccgcaataatttggacggatttacctataatcttgctgtttatttttccatctttaaataaagttgatctaaacttttctactgtttcctcatcttctacatagaatgccccattttctacaaaagctgctattaccttatcataatcttcagcaggagctataactgattgctctgaagaacatataattccattgtcatattttcttcctgttattatatcctgtgcagctttgttataatcgtatcccttatcaactattacctgtgaatttccgtgtccaacgccataagctggtcttccactggagtaagcagctttaactcttccagcaccgcctgtagcaataactacatcagcactttccataagttccttagcagcttctcttgatggtgcttctactatctgtatgatattttctggtgctcccaattttttaagctcagcattcataagttctacagtatgagctgaaactttctttgctttaggatgtggtgctactattattgtatttctgccctttatagcagccattgcattacacataggagttaccactggattagttataggcgtagtagctgccacaactccctttggcttagcaacataaacaagtgccctttcaggttcttcttttattatgcctacagttttcttgtcttttatatgattccaaatagctcctgatttcaaatgacatttagctactttatcttcataaacacccatttctgtttcttcaactgcttcttttgcaaacatttctgcattatcataaaccacttttcctagtgcttttactagtacatcaacttgttcttgactataggcttccaatttttttgtgccacctttgccttttcaattaattcttttatagatacttcattactcatatggctgccgcgcggcaccaggccgctgctgtgatgatgatgatgatggctgctgcccatggtatatctccttcttaaagttaaacaaaattatttctagaggggaattgttatccgctcacaattcccctatagtgagtcgtattaatttcgcgggatagggcaaacaatacaacaatagcgtgcgtggttagcacgctatcgattatttcagaacagttttcagtgcttcaccgatatccgccaggctgcgaacggttttcacgcctgcggcttccagagcagcgaatttctcatccgcagtccctttcccaccggcaatgatggcacccgcgtggcccatacgtttgcctttcggcgcagtcacaccagcgatgtaacccacaactggcttggtaacgtgctctttgatgtacgcagctgcttcttcttcagcgctaccgccgatctcaccgatcatcacgatcgcttcggtctgcggatctttttcgaacatttcgagaatgtcgataaagttagagcccgggatcgggtcaccgccgataccgacacaggtcgactgaccgaaaccgtaatccgtggtctgtttaaccgcttcataggtcagtgtaccggaacgggaaacgatacccactttacccggtttgtgaatgtgaccaggctggataccgattttgcattcacccggagtgataacgcctgggcagttcgggccgatcatacgaacgcctgcttcatccagcttcactttcacggtcagcatatccagcgtcgggatgccttcagtgatggtgataatcagtttgatgcctgcgtcgatggcttccagaatggagtctttgcagaacggtgctggtacgtagataacagaagcggtagcgccagtggcagcaacggcttcacgcacggtgttgaacaccggcaggccgaggtgggtggtgccgcctttacctggggttacgccgccaaccattttagtgccgtatgcaatggcctgttctgagtggaaagtcccctggctaccggtaaagccctggcagataaccttggtgtttttatcgattaaaatggacattatttcccctccactgcggcaacaacctgctgagctgcatccgtcagaccttttgctgcaataatattcaggccgctgtcagccagtttcttcgcgccgagttcggcgttgttaccttccagacgtaccacgaccggtacgttaacacccacttctgctaccgcgccgatgataccgtcagcgatcaggtcgcaacgaacgataccgccgaagatgttaaccagaacggctttcactttgtcgtcagagaggatgattttgaacgcttcggttacacgttctttggttgcgccgccgccaacgtcaaggaagttagccggttcgccgccgtgcagtttaacgatgtccatcgtacccatcgccagacctgcgccgttaaccatacaaccgatgttaccgtccagcgcaacgtagttcagttcccactgtgcagcctgtgcttcacgcggatcttcctgcgactggtcacgcatttcgcgcagatcaggctggcggaacagtgcgttgccgtcagcgcccagtttgccgtcgaggcaaatcagatcgccctgtttggtgatgaccagcgggttgatttcgatcaacgccaggtcgcgctccaggaaaatggtcgccaggcccatgaagattttggtgaactgctgaaccagtttaccttccagacccagtttgaacgccagctcgcgtccctgatacggcatcgggccagtcagcggatcaagcgcaactttatggatcaggtgcggagtttcttccgccactttttcgatttccacgccgccttcggtggaggccataaagaccacacgacgggaactacggtcaacaacggcaccgagatacagctctttagcgatatcggtcgctgcttcaaccagaatctggttaaccggttggccattggcatctgtttgatacgttaccagacgcttgcccagccagttttctgcaaaagcacggatgtcttctttgctgtttacaactttcacaccgcccgctttaccgcggccaccagcgtgaacctgacatttcactacccacggaccggcaccgatttttgaagcggcttcttctgcttcgcgcggagtagtacaggcataacccaccggtgctggtaagccatagcgggcaaaaagttgttttgcctgatattcatgtaagttcatcatggttaattcctcctgttagcccaaaaaacgggtatggagaaacagtagagagttgcgataaaaagcgtcaggtaggatccgctaatcttatggataaaaatgctatggcatagcaaagtgtgacgccgtgcaaataatcaatgtggacttttctgccgtgattatagacacttttgttacgcgtttttgtcatggctttggtcccgctttgttacagaatgcttttaataagcggggttaccggtttggttagcgagaagagccagtaaaagacgcagtgacggcaatgtctgatgcaatatggacaattggtttcttctctgaatggcgggagtatgaaaagtatggctgaagcgcaaaatgatcccctgctgccgggatactcgtttaatgcccatctggtggcgggtttaacgccgattgaggccaacggttatctcgatttttttatcgaccgaccgctgggaatgaaaggttatattctcaatctcaccattcgcggtcagggggtggtgaaaaatcagggacgagaatttgtttgccgaccgggtgatattttgctgttcccgccaggagagattcatcactacggtcgtcatccggaggctcgcgaatggtatcaccagtgggtttactttcgtccgcgcgcctactggcatgaatggcttaactggccgtcaatatttgccaatacggggttctttcgcccggatgaagcgcaccagccgcatttcagcgacctgtttgggcaaatcattaacgccgggcaaggggaagggcgctattcggagctgctggcgataaatctgcttgagcaattgttactgcggcgcatggaagcgattaacgagtcgctccatccaccgatggataatcgggtacgcgaggcttgtcagtacatcagcgatcacctggcagacagcaattttgatatcgccagcgtcgcacagcatgtttgcttgtcgccgtcgcgtctgtcacatcttttccgccagcagttagggattagcgtcttaagctggcgcgaggaccaacgtatcagccaggcgaagctgcttttgagcaccacccggatgcctatcgccaccgtcggtcgcaatgttggttttgacgatcaactctatttctcgcgggtatttaaaaaatgcaccggggccagcccgagcgagttccgtgccggttgtgaagaaaaagtgaatgatgtagccgtcaagttgtcataaagatatagtgagacctctagaagcggccgcgaattccagaaatcatccttagcgaaagctaaggatttttttatctgaaattctgcctcgtgatacgcctatttttataggttaatgtcatgataataatggtttcttagacgtcaggtggcactcgagttgatcgggcacgtaagaggttccaactttcaccataatgaaataagatcactaccgggcgtattttttgagttatcgagattttcaggagctaaggaagctaaaatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttccctggagagagcgagattctccgcgctgtcagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaa

SEQ ID NO: 52 - DNA, 8325 bp, synthetic construct
  source 1..8325: mol_type = other DNA
ccggctatcccgcgaaattaatacgactcactataggggaattgtgagcggataacaattcccctctagaaataattttgtttaactttaagaaggagatataccatgggcagcagccatcatcatcatcatcacagcagcggcctggtgccgcgcggcagccatatgagccgtccgcctcccccgcgtgtagcctctgttcttggcacaatggagatggggcgtagaatggatgccccggcatcagccgccgccgttcgggccttcctcgaaaggggtcataccgagttggatacggctttcatgtactccgacggacagagtgaaactattttggggggtctgggattgggccttggtggcggagattgtcgtgtgaagattgctaccaaggcgaacccttgggacgggaaaagtttaaagcccgattcggtccgcagccagttggaaacaagtctgaagcgcttacaatgcccacaggtcgatctgttttacttgcacgctcccgatcatggcacccctgttgaagaaactcttcatgcttgtcaacggttacatcaggaaggtaaatttgttgaactaggcctgtctaattatgctagttgggaagtagctgaaatctgtaccctgtgtaaatccaatggctggatcttaccaaccgtatatcagggcatgtataatgcaacaactcgccaagtggagacagaattattcccgtgtcttcgtcatttcggtttacggttttacgcatacaacccactggcaggcggtttactaacaggcaaatacaagtatgaggataaagacggaaaacagccggtcggccgcttttttggcaacagctgggcagaaacttatcgtaatcgtttttggaaagagcaccattttgaagccattgctctggtggagaaagcccttcaggctgcctatggagccagtgcgccgtctgtcaccagtgccgccttgcggtggatgtatcaccactcacagttacagggagcccatggtgatgcggttatacttggaatgtcttcccttgaacagctcgagcagaacttggcagccacagaggaaggtcctcttgagcccgctgttgtggacgcttttaatcaggcgtggcatttagtggcccatgaatgcccaaattacttccggtaatcgatagcgtgcgaacagcacgctattgttgtattgttgaagtcgatagcgtgcgaacagcacgctattgttgtattgttgaaggaaggtacagagaccctgcagtccggcaaaaaagggcaaggtgtcaccaccctgccctttttctttaaaaccgaaaagattacttcgcgttatgcaggcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccacaggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagctcgaggcttggattctcaccaataaaaaacgcccggcggcaaccgagcgttctgaacaaatccagatggagttctgaggtcattactggatctatcaacaggagtccaagcgagctcgatatcaaattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctgacagcgcggagaatctcgctctctccagggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcattttagcttccttagctcctgaaaatctcgataactcaaaaaatacgcccggtagtgatcttatttcattatggtgaaagttggaacctcttacgtgcccgatcaactcgagtgccacctgacgtctaagaaaccattattatcatgacattaacctataaaaataggcgtatcacgaggcagaatttcagataaaaaaatccttagctttcgctaaggatgatttctggaattcgcggccgcttctagaggtctcactatatctttatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagagaagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgttttttgggctaacaggaggaattaaccatgatgaacttacatgaatatcaggcaaaacaactttttgcccgctatggcttaccagcaccggtgggttatgcctgtactactccgcgcgaagcagaagaagccgcttcaaaaatcggtgccggtccgtgggtagtgaaatgtcaggttcacgctggtggccgcggtaaagcgggcggtgtgaaagttgtaaacagcaaagaagacatccgtgcttttgcagaaaactggctgggcaagcgtctggtaacgtatcaaacagatgccaatggccaaccggttaaccagattctggttgaagcagcgaccgatatcgctaaagagctgtatctcggtgccgttgttgaccgtagttcccgtcgtgtggtctttatggcctccaccgaaggcggcgtggaaatcgaaaaagtggcggaagaaactccgcacctgatccataaagttgcgcttgatccgctgactggcccgatgccgtatcagggacgcgagctggcgttcaaactgggtctggaaggtaaactggttcagcagttcaccaaaatcttcatgggcctggcgaccattttcctggagcgcgacctggcgttgatcgaaatcaacccgctggtcatcaccaaacagggcgatctgatttgcctcgacggcaaactgggcgctgacggcaacgcactgttccgccagcctgatctgcgcgaaatgcgtgaccagtcgcaggaagatccgcgtgaagcacaggctgcacagtgggaactgaactacgttgcgctggacggtaacatcggttgtatggttaacggcgcaggtctggcgatgggtacgatggacatcgttaaactgcacggcggcgaaccggctaacttccttgacgttggcggcggcgcaaccaaagaacgtgtaaccgaagcgttcaaaatcatcctctctgacgacaaagtgaaagccgttctggttaacatcttcggcggtatcgttcgttgcgacctgatcgctgacggtatcatcggcgcggtagcagaagtgggtgttaacgtaccggtcgtggtacgtctggaaggtaacaacgccgaactcggcgcgaagaaactggctgacagcggcctgaatattattgcagcaaaaggtctgacggatgcagctcagcaggttgttgccgcagtggaggggaaataatgtccattttaatcgataaaaacaccaaggttatctgccagggctttaccggtagccaggggactttccactcagaacaggccattgcatacggcactaaaatggttggcggcgtaaccccaggtaaaggcggcaccacccacctcggcctgccggtgttcaacaccgtgcgtgaagccgttgctgccactggcgctaccgcttctgttatctacgtaccagcaccgttctgcaaagactccattctggaagccatcgacgcaggcatcaaactgattatcaccatcactgaaggcatcccgacgctggatatgctgaccgtgaaagtgaagctggatgaagcaggcgttcgtatgatcggcccgaactgcccaggcgttatcactccgggtgaatgcaaaatcggtatccagcctggtcacattcacaaaccgggtaaagtgggtatcgtttcccgttccggtacactgacctatgaagcggttaaacagaccacggattacggtttcggtcagtcgacctgtgtcggtatcggcggtgacccgatcccgggctctaactttatcgacattctcgaaatgttcgaaaaagatccgcagaccgaagcgatcgtgatgatcggtgagatcggcggtagcgctgaagaagaagcagctgcgtacatcaaagagcacgttaccaagccagttgtgggttacatcgctggtgtgactgcgccgaaaggcaaacgtatgggccacgcgggtgccatcattgccggtgggaaagggactgcggatgagaaattcgctgctctggaagccgcaggcgtgaaaaccgttcgcagcctggcggatatcggtgaagcactgaaaactgttctgaaataatcgatagcgtgctaaccacgcacgctattgttgtattgtttgccctatcccgcgaaattaatacgactcactataggggaattgtgagcggataacaattcccctctagaaataattttgtttaactttaagaaggagatataccatgggcagcagccatcatcatcatcatcacagcagcggcctggtgccgcgcggcagccatatgagtaatgaagtatctataaaagaattaattgaaaaggcaaaggtggcacaaaaaaattggaagcctatagtcaagaacaagttgatgtactagtaaaagcactaggaaaagtggtttatgataatgcagaaatgtttgcaaaagaagcagttgaagaaacagaaatgggtgtttatgaagataaagtagctaaatgtcatttgaaatcaggagctatttggaatcatataaaagacaagaaaactgtaggcataataaaagaagaacctgaaagggcacttgtttatgttgctaagccaaagggagttgtggcagctactacgcctataactaatccagtggtaactcctatgtgtaatgcaatggctgctataaagggcagaaatacaataatagtagcaccacatcctaaagcaaagaaagtttcagctcatactgtagaacttatgaatgctgagcttaaaaaattgggagcaccagaaaatatcatacagatagtagaagcaccatcaagagaagctgctaaggaacttatggaaagtgctgatgtagttattgctacaggcggtgctggaagagttaaagctgcttactccagtggaagaccagcttatggcgttggacacggaaattcacaggtaatagttgataagggatacgattataacaaagctgcacaggatataataacaggaagaaaatatgacaatggaattatatgttcttcagagcaatcagttatagctcctgctgaagattatgataaggtaatagcagcttttgtagaaaatggggcattctatgtagaagatgaggaaacagtagaaaagtttagatcaactttatttaaagatggaaaaataaacagcaagattataggtaaatccgtccaaattattgcggatcttgcaggagtaaaagtaccagaaggtactaaggttatagtacttaagggtaaaggtgcaggagaaaaagatgtactttgtaaagaaaaaatgtgtccagttttagtagcattgaaatatgatacttttgaagaagcagttgaaatagctatggctaattatatgtatgaaggagctggtcatacagcaggcatacattctgacaatgacgagaacataagatatgcaggaactgtattacctataagcagattagttgtaaatcagcctgcaactactgctggaggaagtttcaataatggatttaaccctactactacactaggctgcggatcatggggcagaaacagtatttcagaaaatcttacttacgagcatcttataaatgtttcaagaatagggtatttcaataaagaagcaaaagttcctagctatgaggaaatatggggataatcgatagcgtgctaaccacgcacggcattgttgtattgtt

SEQ ID NO: 53 - DNA, 996 bp, Pseudomonas aeruginosa
  source 1..996: mol_type = other DNA
atgttcaagacgatggtacgggcgctgttgtgcgccctggcgatcggtggctcctgcctggcgaatgccgcgcagccgatcgtcatcaagttctcccacgtggtcgcggaaaacacgcccaaaggccagggcgcgctgctgttcaagaaactcgtcgaacagcgcctgggcggccgggtcgaggtagacgtctatcccaactcgtcgctgttcggcgacggcaaggaaatggaggcgctgctgctgggcgacgtgcagatgctggcgccgtccctggccaagttcgagcagtacacgcgcaaggtgcagatcttcgacctgccgttcctcttcgacgacatccaggcggtcgaccgcttccagcgcagcccgcaggggcgcgccttgctgaccagcatgcagggcaagggcatcctcggcctggcctactggcacaacgggatgaagcagctctccgccaatcgcccgttgctggagccggaagacgcgcgcgggctgaaattccgcgtgcaggcgtccgacgtgctcaacgagcagttccgccagctccgcgcgatctcccggaagatgtccttcgccgaggtctaccagggcttgcagaccggcgtggtgaatggcaccgagaacacctggtcgaactacgagagccagaaggtcaacgaggtacagaagtatttcaccgagtccaaccacggcttggtggactacatggtgatcaccaacgcgaagttctggaatggccttccggcggacatccgcgaagaactgcagcggatcatggacgaggtcaccgtgcaggtgaacctcgaggccgagcgcctcaaccgcgacgcccgccagcgcatcctcgccagtggcgccagcgagatccataccctgagcccgcagcagcgcgccgactggcgccaggcgatgcagccggtatggcagaaattccgcggcaatgtcggtgccgatctgctccaggccgccgaagcttccaaccgtcccgattga

SEQ ID NO: 54 - DNA, 639 bp, Pseudomonas aeruginosa
  source 1..639: mol_type = other DNA
atgaatcgactcgtgcgcgtctggaaccgcttcgaggagggcgctatcgccttcctgctggcggcgatgacgctggtgaccttcctctatgtggtgatgaccaacgtcttcaccctgttctacgacctcggcgaccgcctgccgacactgcgggctccgctgtactcgatgggcgacgcgatcctcggggtagcccaggatatgtcctggagcaacgcgctgaccaaggcgctgttcggctggctgatcttcctcggcatcgcctacggcgtacgcaccgccggccacctcggggtggatgccctggtgcgcctggcgccgcgtcccttgcagcgcctgatcgggatactcgcctgcctctgctgcctgggctacgccgggctgttcatggttgccagctacgactgggtgaagaccctgttcgtcgccggcatcggcgccgaggacctcgaccacttcggcatcctccaggcctacatcgcggtggtggtgccggtcggcttcggcctggtgctgctgcgctacctggaaatcctcgtcaacctgttgcgcggtcgccagctcgggctgggcctcgccgacgaggccgccgaggcctccaaactggccggcgaagaggccggggagaaccgtccatga

SEQ ID NO: 55 - DNA, 1281 bp, Pseudomonas aeruginosa
  source 1..1281: mol_type = other DNA
atgaccgtgctgatcctgttcctgctgctgttcctgttcatgttcatcggcgtgcccatcgccatctccctggggctgtccggcgccctgacgatcctgctgttcagcccggattcggtgcgctcgctggcgatcaagctgttcgagacctccgaggcctacaccctgctggcgatcccgttcttcctgctctccggcgcgttcatgaccaccggtggcgtggcgcggcgcctgatcgacttcgccaatgcctgcgtcgggcatatccgcggcggcctggcgatcgctgcggtgcttgcctgcatgctgttcgccgcgctgtccggttcgtcgccggcgacagtggcggcggtcggctcgatcgctgtggccggcatggtccgctccggctatccgcgcgccttcggcgcggggatcatctgcaatgccgggaccctgggcatcctcatcccgccatcgatcgtgatggtggtctacgccgccgccaccgagacctcggtgggcaagctgttcgtcgccggggtggttccggggctgctgctcggggtgatcctgatggtggtgatctacatcgtcgcgcgggtcaagaagctgccggcgcagccgcgcgccagcttccgcgagtggctggccgccgcacgccgtgcaggctggggcctgttgttgctggtgatcatcctcggcggcatctattccgggctgttcacccccaccgaggcggcggcggtggcggcggtgtattcggccttcgtcgcgctgttcgtgtacaaggacatgcgcctgcgcgactgtcccaaggtgctgctggagtcggggcggctgagcatcatgctgatgttcatcatcgccaatgccatgctcttcgcccacgtgttgaccaccgagcagatcccccagcagatcaccgaatgggtggtgcaggaaggcctgaccccgatcggcttcctgctgatggtcaacgtggtgctgctggtcgccggcagcttcatggagccctcggcgatcgtgctgatcctggcgccgatcttcttccccatcgccatgaagctgggcatcgacccgatccacctgggcatcgtcatggtggtgaacatggagatcggcctggtacacccgccggtcggcctcaacctgttcgtcacttcggcggtcaccggcatgccgctgggggcgaccatccgcgcggcgctgccgtggctgatgatcctgctgctgttcctgatcctggtgacctacgtgccgttgatctcgctgtggctgccgggcctgctgggcatgagc

SEQ ID NO: 56 - protein, 331 aa, Pseudomonas aeruginosa
MFKTMVRALLCALAIGGSCLANAAQPIVIKFSHVVAENTPKGQGALLFKKLVEQRLGGRVEVDVYPNSSLFGDGKEMEALLLGDVQMLAPSLAKFEQYTRKVQIFDLPFLFDDIQAVDRFQRSPQGRALLTSMQGKGILGLAYWHNGMKQLSANRPLLEPEDARGLKFRVQASDVLNEQFRQLRAISRKMSFAEVYQGLQTGVVNGTENTWSNYESQKVNEVQKYFTESNHGLVDYMVITNAKFWNGLPADIREELQRIMDEVTVQVNLEAERLNRDARQRILASGASEIHTLSPQQRADWRQAMQPVWQKFRGNVGADLLQAAEASNRPD

SEQ ID NO: 57 - protein, 212 aa, Pseudomonas aeruginosa
MNRLVRVWNRFEEGAIAFLLAAMTLVTFLYVVMTNVFTLFYDLGDRLPTLRAPLYSMGDAILGVAQDMSWSNALTKALFGWLIFLGIAYGVRTAGHLGVDALVRLAPRPLQRLIGILACLCCLGYAGLFMVASYDWVKTLFVAGIGAEDLDHFGILQAYIAVVVPVGFGLVLLRYLEILVNLLRGRQLGLGLADEAAEASKLAGEEAGENRP

SEQ ID NO: 58 - protein, 427 aa, Pseudomonas aeruginosa
MTVLILFLLLFLFMFIGVPIAISLGLSGALTILLFSPDSVRSLAIKLFETSEAYTLLAIPFFLLSGAFMTTGGVARRLIDFANACVGHIRGGLAIAAVLACMLFAALSGSSPATVAAVGSIAVAGMVRSGYPRAFGAGIICNAGTLGILIPPSIVMVVYAAATETSVGKLFVAGVVPGLLLGVILMVVIYIVARVKKLPAQPRASFREWLAAARRAGWGLLLLVIILGGIYSGLFTPTEAAAVAAVYSAFVALFVYKDMRLRDCPKVLLESGRLSIMLMFIIANAMLFAHVLTTEQIPQQITEWVVQEGLTPIGFLLMVNVVLLVAGSFMEPSAIVLILAPIFFPIAMKLGIDPIHLGIVMVVNMEIGLVHPPVGLNLFVTSAVTGMPLGATIRAALPWLMILLLFLILVTYVPLISLWLPGLLGMS

SEQ ID NO: 59 - protein, 430 aa, Yersinia rohdei
MDAFILVFTLAILLAIGVPVAYAVGLSAIAGAFWIDLPLEAVMIQITSGVNKFSLLAIPFFILAGAIMAEGGIARRLVNFAYIFVGFIRGGLSLVNIVASTFFGAISGSSVADTASIGSVMIPEMDKKGYPRDFAAAVTASGSVQAILTPPSHNSVIYSLATGGTVSIAALFIAGILPGLLLSFTLMLMCVGFAYKRGYPKGERVPFRQALKIFLDTLWGLMTVVIIMGGILSGIFTATESAAIACLWSFFVTMFIYRDYKWSELPKLMYRTVKTVTIVMILIGFAASFGAIMTYMQLPERITEFFTSVSDNKYVILMCINIMLLLVGTLMDMAPLILILTPVLMPVTNSLGIDPVHFGMIMLINLGIGLITPPVGSVLFVASAVSKQKIEQVVKAMLPFYGALLLVLMLVTYIPEISLWLPRLFGVYNG

SEQ ID NO: 60 - protein, 454 aa, Tulipa sp.
MVVQGSHVLIVPYPGQGHLNPMLQFGKHLAHHGLTVTVATTRYILSTNPPDTKLSHITFAPISDGFDDGGFGACGDVTSYLARMESVGSQSLTDLIESSSAEGRPVRVMVYEPFLPWALDVGKRLGITCAAFFTQSCAVDAIYSHVRDGKLSLPAEEAVELPGLPRLEPNDLPSYFTDPVPGPYQAYFEMLVQQLSNLERADIVLINSFYELEIRELDWLKTSWPVTTVGPTVPSKYLDNEIPSDGHYGFNLFTPDTGPCMSWLDSQTPNSVVYVSYGSMSDINSKQTEEIARALQNSGKNFLWVVRKTEMNKVPIDFINETANQGLVVSWSPQLEVLAHPSVGCFVTHCGWNSTTEGLSLGVPMIGVPQWTDQQTNAKYVEDVWKVGLRVKVDEKRFLKRDELERCIREVMEGDKSEEIRKNAGKWREMAKIAVSKGGSSNKNILEFIEKYCS

SEQ ID NO: 61 - protein, 388 aa, Escherichia coli
MGKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELVKDPRIAATMENAQKGEIMPNIPQMSAFWYAVRTAVINAASGRQTVDEALKDAQTNSSSNNNNNNNNNNLGIEGR

SEQ ID NO: 62 - protein, 858 aa, synthetic construct
MGHHHHHHHHHHSSGKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELVKDPRIAATMENAQKGEIMPNIPQMSAFWYAVRTAVINAASGRQTVDEALKDAQTNSSSNNNNNNNNNNLGIEGRHGSRVVQGSHVLIVPYPGQGHLNPMLQFGKHLAHHGLTVTVATTRYILSTNPPDTKLSHITFAPISDGFDDGGFGACGDVTSYLARMESVGSQSLTDLIESSSAEGRPVRVMVYEPFLPWALDVGKRLGITCAAFFTQSCAVDAIYSHVRDGKLSLPAEEAVELPGLPRLEPNDLPSYFTDPVPGPYQAYFEMLVQQLSNLERADIVLINSFYELEIRELDWLKTSWPVTTVGPTVPSKYLDNEIPSDGHYGFNLFTPDTGPCMSWLDSQTPNSVVYVSYGSMSDINSKQTEEIARALQNSGKNFLWVVRKTEMNKVPIDFINETANQGLVVSWSPQLEVLAHPSVGCFVTHCGWNSTTEGLSLGVPMIGVPQWTDQQTNAKYVEDVWKVGLRVKVDEKRFLKRDELERCIREVMEGDKSEEIRKNAGKWREMAKIAVSKGGSSNKNILEFIEKYCS

SEQ ID NO: 63 - DNA, 10007 bp, synthetic construct
  source 1..10007: mol_type = other DNA
catatgaccaaacagagcgcagatagcaatgcaaaaagcggtgttaccagcgaaatttgtcattgggcaagcaatctggcaaccgatgatattccgagtgatgttctggaacgtgccaaatatctgattctggatggtattgcatgtgcatgggttggtgcacgtgttccgtggtcagaaaaatatgttcaggcaaccatgagctttgaaccgcctggtgcatgtcgtgttattggttatggccagaaactgggtccggttgcagcagcaatgaccaatagcgcatttattcaggccaccgaactggatgattatcatagcgaagcaccgctgcatagcgcaagcattgttctgcctgcagtttttgcagcaagcgaagttctggcagaacagggtaaaaccattagcggtattgatgttattctggcagccattgttggttttgaaagcggtccgcgtattggtaaagcaatttatggtagcgatctgctgaataatggttggcattgtggtgcagtttatggtgcaccggcaggcgcactggccaccggtaaactgctgggtctgacaccggatagcatggaagatgcactgggtattgcctgtacccaggcatgtggtctgatgagcgcacagtatggtggtatggttaaacgtgttcagcatggttttgcagcccgtaatggtctgctgggtggcctgctggcacatggtggttatgaagcaatgaaaggtgtgctggaacgtagctatggtggttttctgaaaatgtttaccaaaggcaatggtcgtgaacctccgtataaagaagaagaagttgttgcaggtctgggtagcttttggcatacctttaccattcgcattaaactgtatgcatgttgtggtctggttcatggtccggtggaagcaattgaaaatctgcagggtcgttatccggaactgctgaatcgtgcaaatctgagcaatattcgtcatgttcatgttcagctgagcaccgcaagcaatagccattgcggttggattccggaagaacgtccgattagcagcattgcaggtcagatgagcgttgcatatattctggccgttcagctggttgatcagcagtgtctgctgagccagtttagcgaatttgatgataacctggaacgtccggaagtttgggatctggcacgtaaagttaccagcagccagagcgaagaatttgatcaggatggtaattgtctgagcgcaggtcgtgttcgtattgaatttaatgatggttccagcattaccgaaagcgttgaaaaaccgctgggtgttaaagaaccgatgccgaatgaacgtatcctgcataaatatcgtaccctggcaggtagcgttaccgatgaaagccgtgtgaaagaaattgaagatctggttctgggtctggatcgtctgaccgatattagtccgctgctggaactgctgaactgtccggttaaaagtccgctggtttaaggtaccaggaggacagctatgtttgaacgcgatattgttgccaccgataataacaaagcagttctgcattatccgggtggtgaatttgaaatggatatcattgaagccagcgaaggtaataatggtgttgttctgggtaaaatgctgagcgaaaccggtctgattacctttgatccgggttatgttagcaccggtagcaccgaaagtaaaatcacctatattgatggtgatgccggtattctgcgttatcgcggttatgatattgcagatctggccgaaaatgccacctttaatgaagttagttacctgctgattaacggtgaactgccgactccggatgaactgcacaaatttaacgatgaaattcgccatcataccctgctggatgaagatttcaaaagtcagtttaacgtgtttccgcgtgatgcacatccgatggcaaccctggccagcagcgttaatattctgagcacctattatcaggatcagctgaatccgctggatgaggcacagctggataaagcaaccgttcgtctgatggcaaaagttccgatgctggcagcatatgcacatcgtgcccgtaaaggtgcaccgtatatgtatccagataatagcctgaatgcccgtgaaaactttctgcgtatgatgtttggttatccgaccgaaccgtatgaaattgatccgattatggtgaaagcactggacaaactgctgattctgcatgccgatcatgaacagaattgtagcaccagcaccgttcgcatgattggtagcgcacaggcaaatatgtttgttagcattgccggtggtattaatgcactgagtggtccgctgcatggtggtgcaaatcaggcagttctggaaatgctggaagatatcaaatcaaatcatggcggtgatgccaccgagtttatgaataaagtgaaaaacaaagaggatggcgtccgcctgatgggttttggtcatcgtgtgtataaaaactatgatccgcgtgccgcaatcgttaaagaaaccgcacatgaaattctggaacatctgggtggtgatgatctgctggatctggcgattaaactggaagaaattgcactggcagatgactattttatcagccgcaaactgtatccgaacgtggatttctatacaggtctgatttatcgtgcaatgggctttccgaccgattttttcaccgttctgtttgcaattggtcgtctgcctggttggattgcacattatcgtgaacagctgggtgcagcaggcaataaaatcaatcgtccgcgtcaggtttataccggtaatgaaagtcgtaaactggttcctcgtgaagaacgttaatcgatagcgtgcgaacagcacgctattgttgtattgttttaggtacagagaccctgcagtccggcaaaaaagggcaaggtgtcaccaccctgccctttttctttaaaaccgaaaagattacttcgcgttatgcaggcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccacaggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagctcgaggcttggattctcaccaataaaaaacgcccggcggcaaccgagcgttctgaacaaatccagatggagttctgaggtcattactggatctatcaacaggagtccaagcgagctcgatatcaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgacacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgcccgatcaactcgagtgccacctgacgtctaagaaaccattattatcatgacattaacctataaaaataggcgtatcacgaggcagaatttcagataaaaaaaatccttagctttcgctaaggatgatttctggaattcgcggccgcttctagaggtctcactatatctctatcccgcgaaattaatacgactcactataggggaattgtgagcggataacaattcccctctagaaataattttgtttaactttaagaaggagatataccatgggcagcagccatcatcatcatcatcacagcagcggcctggtgccgcgcggcagccatatgaacttacatgaatatcaggcaaaacaactttttgcccgctatggcttaccagcaccggtgggttatgcctgtactactccgcgcgaagcagaagaagccgcttcaaaaatcggtgccggtccgtgggtagtgaaatgtcaggttcacgctggtggccgcggtaaagcgggcggtgtgaaagttgtaaacagcaaagaagacatccgtgcttttgcagaaaactggctgggcaagcgtctggtaacgtatcaaacagatgccaatggccaaccggttaaccagattctggttgaagcagcgaccgatatcgctaaagagctgtatctcggtgccgttgttgaccgtagttcccgtcgtgtggtctttatggcctccaccgaaggcggcgtggaaatcgaaaaagtggcggaagaaactccgcacctgatccataaagttgcgcttgatccgctgactggcccgatgccgtatcagggacgcgagctggcgttcaaactgggtctggaaggtaaactggttcagcagttcaccaaaatcttcatgggcctggcgaccattttcctggagcgcgacctggcgttgatcgaaatcaacccgctggtcatcaccaaacagggcgatctgatttgcctcgacggcaaactgggcgctgacggcaacgcactgttccgccagcctgatctgcgcgaaatgcgtgaccagtcgcaggaagatccgcgtgaagcacaggctgcacagtgggaactgaactacgttgcgctggacggtaacatcggttgtatggttaacggcgcaggtctggcgatgggtacgatggacatcgttaaactgcacggcggcgaaccggctaacttccttgacgttggcggcggcgcaaccaaagaacgtgtaaccgaagcgttcaaaatcatcctctctgacgacaaagtgaaagccgttctggttaacatcttcggcggtatcgttcgttgcgacctgatcgctgacggtatcatcggcgcggtagcagaagtgggtgttaacgtaccggtcgtggtacgtctggaaggtaacaacgccgaactcggcgcgaagaaactggctgacagcggcctgaatattattgcagcaaaaggtctgacggatgcagctcagcaggttgttgccgcagtggaggggaaataatgtccattttaatcgataaaaacaccaaggttatctgccagggctttaccggtagccaggggactttccactcagaacaggccattgcatacggcactaaaatggttggcggcgtaaccccaggtaaaggcggcaccacccacctcggcctgccggtgttcaacaccgtgcgtgaagccgttgctgccactggcgctaccgcttctgttatctacgtaccagcaccgttctgcaaagactccattctggaagccatcgacgcaggcatcaaactgattatcaccatcactgaaggcatcccgacgctggatatgctgaccgtgaaagtgaagctggatgaagcaggcgttcgtatgatcggcccgaactgcccaggcgttatcactccgggtgaatgcaaaatcggtatccagcctggtcacattcacaaaccgggtaaagtgggtatcgtttcccgttccggtacactgacctatgaagcggttaaacagaccacggattacggtttcggtcagtcgacctgtgtcggtatcggcggtgacccgatcccgggctctaactttatcgacattctcgaaatgttcgaaaaagatccgcagaccgaagcgatcgtgatgatcggtgagatcggcggtagcgctgaagaagaagcagctgcgtacatcaaagagcacgttaccaagccagttgtgggttacatcgctggtgtgactgcgccgaaaggcaaacgtatgggccacgcgggtgccatcattgccggtgggaaagggactgcggatgagaaattcgctgctctggaagccgcaggcgtgaaaaccgttcgcagcctggcggatatcggtgaagcactgaaaactgttctgaaataatcgatgccgggagaacatcccggcattgttgtattgtttgccctatcccgcgaaattaatacgactcactataggggaattgtgagcggataacaattcccctctagaaataattttgtttaactttaagaaggagatataccatgggcagcagccatcatcatcatcatcacagcagcggcctggtgccgcgcggcagccatatgagtaatgaagtatctataaaagaattaattgaaaaggcaaaggtggcacaaaaaaaattggaagcctatagtcaagaacaagttgatgtactagtaaaagcactaggaaaagtggtttatgataatgcagaaatgtttgcaaaagaagcagttgaagaaacagaaatgggtgtttatgaagataaagtagctaaatgtcatttgaaatcaggagctatttggaatcatataaaagacaagaaaactgtaggcataataaaagaagaacctgaaagggcacttgtttatgttgctaagccaaagggagttgtggcagctactacgcctataactaatccagtggtaactcctatgtgtaatgcaatggctgctataaagggcagaaatacaataatagtagcaccacatcctaaagcaaagaaagtttcagctcatactgtagaacttatgaatgctgagcttaaaaaattgggagcaccagaaaatatcatacagatagtagaagcaccatcaagagaagctgctaaggaacttatggaaagtgctgatgtagttattgctacaggcggtgctggaagagttaaagctgcttactccagtggaagaccagcttatggcgttggacctggaaattcacaggtaatagttgataagggatacgattataacaaagctgcacaggatataataacaggaagaaaatatgacaatggaattatatgttcttcagagcaatcagttatagctcctgctgaagattatgataaggtaatagcagcttttgtagaaaatggggcattctatgtagaagatgaggaaacagtagaaaagtttagatcaactttatttaaagatggaaaaataaacagcaagattataggtaaatccgtccaaattattgcggatcttgcaggagtaaaagtaccagaaggtactaaggttatagtacttaagggtaaaggtgcaggagaaaaagatgtactttgtaaagaaaaaatgtgtccagttttagtagcattgaaatatgatacttttgaagaagcagttgaaatagctatggctaattatatgtatgaaggagctggtcatacagcaggcatacattctgacaatgacgagaacataagatatgcaggaactgtattacctataagcagattagttgtaaatcagcctgcaactactgctggaggaagtttcaataatggatttaaccctactactacactaggctgcggatcatggggcagaaacagtatttcagaaaatcttacttacgagcatcttataaatgtttcaagaatagggtatttcaataaagaagcaaaagttcctagctatgaggaaatatggggataatcgatgccgggagaacatcccggcattgttgtattgttccggctatcccgcgaaattaatacgactcactataggggaattgtgagcggataacaattcccctctagaaataattttgtttaactttaagaaggagatataccatgggcagcagccatcatcatcatcatcacagcagcggcctggtgccgcgcggcagccatatgagccgtccgcctcccccgcgtgtagcctctgttcttggcacaatggagatggggcgtagaatggatgccccggcatcagccgccgccgttcgggccttcctcgaaaggggtcataccgagttggatacggctttcatgtactccgacggacagagtgaaactattttggggggtctgggattgggccttggtggcggagattgtcgtgtgaagattgctaccaaggcgaacccttgggacgggaaaagtttaaagcccgattcggtccgcagccagttggaaacaagtctgaagcgcttacaatgcccacaggtcgatctgttttacttgcacgctcccgatcatggcacccctgttgaagaaactcttcatgcttgtcaacggttacatcaggaaggtaaatttgttgaactaggcctgtctaattatgctagttgggaagtagctgaaatctgtaccctgtgtaaatccaatggctggatcttaccaaccgtatatcagggcatgtataatgcaacaactcgccaagtggagacagaattattcccgtgtcttcgtcatttcggtttacggttttacgcatacaacccactggcaggcggtttactaacaggcaaatacaagtatgaggataaagacggaaaacagccggtcggccgcttttttggcaacagctgggcagaaacttatcgtaatcgtttttggaaagagcaccattttgaagccattgctctggtggagaaagcccttcaggctgcctatggagccagtgcgccgtctgtcaccagtgccgccttgcggtggatgtatcaccactcacagttacagggagcccatggtgatgcggttatacttggaatgtcttcccttgaacagctcgagcagaacttggcagccacagaggaaggtcctcttgagcccgctgttgtggacgcttttaatcaggcgtggcatttagtggcccatgaatgcccaaattacttccggtaatcgatagcgtgcgaacagcacgctattgttgtattgttgaagctatttgatggctagctcagtccttggtattatgctagcgtacagatctaataattttgtttaactttgggaggata

SEQ ID NO: 64 - DNA, 4321 bp, synthetic construct
  source 1..4321: mol_type = other DNA
cgtctcaatctctatcccgcgaaattaatacgactcactataggggaattgtgagcggataacaattcccctctagaaataattttgtttaactttaagaaggagatataccatgggcagcagccatcatcatcatcatcacagcagcggcctggtgccgcgcggcagccatatgaacttacatgaatatcaggcaaaacaactttttgcccgctatggcttaccagcaccggtgggttatgcctgtactactccgcgcgaagcagaagaagccgcttcaaaaatcggtgccggtccgtgggtagtgaaatgtcaggttcacgctggtggccgcggtaaagcgggcggtgtgaaagttgtaaacagcaaagaagacatccgtgcttttgcagaaaactggctgggcaagcgtctggtaacgtatcaaacagatgccaatggccaaccggttaaccagattctggttgaagcagcgaccgatatcgctaaagagctgtatctcggtgccgttgttgaccgtagttcccgtcgtgtggtctttatggcctccaccgaaggcggcgtggaaatcgaaaaagtggcggaagaaactccgcacctgatccataaagttgcgcttgatccgctgactggcccgatgccgtatcagggacgcgagctggcgttcaaactgggtctggaaggtaaactggttcagcagttcaccaaaatcttcatgggcctggcgaccattttcctggagcgcgacctggcgttgatcgaaatcaacccgctggtcatcaccaaacagggcgatctgatttgcctcgacggcaaactgggcgctgacggcaacgcactgttccgccagcctgatctgcgcgaaatgcgtgaccagtcgcaggaagatccgcgtgaagcacaggctgcacagtgggaactgaactacgttgcgctggacggtaacatcggttgtatggttaacggcgcaggtctggcgatgggtacgatggacatcgttaaactgcacggcggcgaaccggctaacttccttgacgttggcggcggcgcaaccaaagaacgtgtaaccgaagcgttcaaaatcatcctctctgacgacaaagtgaaagccgttctggttaacatcttcggcggtatcgttcgttgcgacctgatcgctgacggtatcatcggcgcggtagcagaagtgggtgttaacgtaccggtcgtggtacgtctggaaggtaacaacgccgaactcggcgcgaagaaactggctgacagcggcctgaatattattgcagcaaaaggtctgacggatgcagctcagcaggttgttgccgcagtggaggggaaataatgtccattttaatcgataaaaacaccaaggttatctgccagggctttaccggtagccaggggactttccactcagaacaggccattgcatacggcactaaaatggttggcggcgtaaccccaggtaaaggcggcaccacccacctcggcctgccggtgttcaacaccgtgcgtgaagccgttgctgccactggcgctaccgcttctgttatctacgtaccagcaccgttctgcaaagactccattctggaagccatcgacgcaggcatcaaactgattatcaccatcactgaaggcatcccgacgctggatatgctgaccgtgaaagtgaagctggatgaagcaggcgttcgtatgatcggcccgaactgcccaggcgttatcactccgggtgaatgcaaaatcggtatccagcctggtcacattcacaaaccgggtaaagtgggtatcgtttcccgttccggtacactgacctatgaagcggttaaacagaccacggattacggtttcggtcagtcgacctgtgtcggtatcggcggtgacccgatcccgggctctaactttatcgacattctcgaaatgttcgaaaaagatccgcagaccgaagcgatcgtgatgatcggtgagatcggcggtagcgctgaagaagaagcagctgcgtacatcaaagagcacgttaccaagccagttgtgggttacatcgctggtgtgactgcgccgaaaggcaaacgtatgggccacgcgggtgccatcattgccggtgggaaagggactgcggatgagaaattcgctgctctggaagccgcaggcgtgaaaaccgttcgcagcctggcggatatcggtgaagcactgaaaactgttctgaaataatcgatgccgggagaacatcccggcattgttgtattgtttgccagagacgctgcaggcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagagccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggcagaatttcagataaaaaaaatccttagctttcgctaaggatgatttctggaattcgcggccgcttctagaa

SEQ ID NO: 65 - DNA, 3647 bp, synthetic construct
  source 1..3647: mol_type = other DNA
cgtctcatgccctatcccgcgaaattaatacgactcactataggggaattgtgagcggataacaattcccctctagaaataattttgtttaactttaagaaggagatataccatgggcagcagccatcatcatcatcatcacagcagcggcctggtgccgcgcggcagccatatgagtaatgaagtatctataaaagaattaattgaaaaggcaaaggtggcacaaaaaaaattggaagcctatagtcaagaacaagttgatgtactagtaaaagcactaggaaaagtggtttatgataatgcagaaatgtttgcaaaagaagcagttgaagaaacagaaatgggtgtttatgaagataaagtagctaaatgtcatttgaaatcaggagctatttggaatcatataaaagacaagaaaactgtaggcataataaaagaagaacctgaaagggcacttgtttatgttgctaagccaaagggagttgtggcagctactacgcctataactaatccagtggtaactcctatgtgtaatgcaatggctgctataaagggcagaaatacaataatagtagcaccacatcctaaagcaaagaaagtttcagctcatactgtagaacttatgaatgctgagcttaaaaaattgggagcaccagaaaatatcatacagatagtagaagcaccatcaagagaagctgctaaggaacttatggaaagtgctgatgtagttattgctacaggcggtgctggaagagttaaagctgcttactccagtggaagaccagcttatggcgttggacctggaaattcacaggtaatagttgataagggatacgattataacaaagctgcacaggatataataacaggaagaaaatatgacaatggaattatatgttcttcagagcaatcagttatagctcctgctgaagattatgataaggtaatagcagcttttgtagaaaatggggcattctatgtagaagatgaggaaacagtagaaaagtttagatcaactttatttaaagatggaaaaataaacagcaagattataggtaaatccgtccaaattattgcggatcttgcaggagtaaaagtaccagaaggtactaaggttatagtacttaagggtaaaggtgcaggagaaaaagatgtactttgtaaagaaaaaatgtgtccagttttagtagcattgaaatatgatacttttgaagaagcagttgaaatagctatggctaattatatgtatgaaggagctggtcatacagcaggcatacattctgacaatgacgagaacataagatatgcaggaactgtattacctataagcagattagttgtaaatcagcctgcaactactgctggaggaagtttcaataatggatttaaccctactactacactaggctgcggatcatggggcagaaacagtatttcagaaaatcttacttacgagcatcttataaatgtttcaagaatagggtatttcaataaagaagcaaaagttcctagctatgaggaaatatggggataatcgatgccgggagaacatcccggcattgttgtattgttccggagagacgctgcaggcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagagccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggcagaatttcagataaaaaaaatccttagctttcgctaaggatgatttctggaattcgcggccgcttctagaa

SEQ ID NO: 66 - DNA, 3278 bp, synthetic construct
  source 1..3278: mol_type = other DNA
cgtctcaccggctatcccgcgaaattaatacgactcactataggggaattgtgagcggataacaattcccctctagaaataattttgtttaactttaagaaggagatataccatgggcagcagccatcatcatcatcatcacagcagcggcctggtgccgcgcggcagccatatgagccgtccgcctcccccgcgtgtagcctctgttcttggcacaatggagatggggcgtagaatggatgccccggcatcagccgccgccgttcgggccttcctcgaaaggggtcataccgagttggatacggctttcatgtactccgacggacagagtgaaactattttggggggtctgggattgggccttggtggcggagattgtcgtgtgaagattgctaccaaggcgaacccttgggacgggaaaagtttaaagcccgattcggtccgcagccagttggaaacaagtctgaagcgcttacaatgcccacaggtcgatctgttttacttgcacgctcccgatcatggcacccctgttgaagaaactcttcatgcttgtcaacggttacatcaggaaggtaaatttgttgaactaggcctgtctaattatgctagttgggaagtagctgaaatctgtaccctgtgtaaatccaatggctggatcttaccaaccgtatatcagggcatgtataatgcaacaactcgccaagtggagacagaattattcccgtgtcttcgtcatttcggtttacggttttacgcatacaacccactggcaggcggtttactaacaggcaaatacaagtatgaggataaagacggaaaacagccggtcggccgcttttttggcaacagctgggcagaaacttatcgtaatcgtttttggaaagagcaccattttgaagccattgctctggtggagaaagcccttcaggctgcctatggagccagtgcgccgtctgtcaccagtgccgccttgcggtggatgtatcaccactcacagttacagggagcccatggtgatgcggttatacttggaatgtcttcccttgaacagctcgagcagaacttggcagccacagaggaaggtcctcttgagcccgctgttgtggacgcttttaatcaggcgtggcatttagtggcccatgaatgcccaaattacttccggtaatcgatagcgtgcgaacagcacgctattgttgtattgttgaagagagacgctgcaggcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagagccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggcagaatttcagataaaaaaaatccttagctttcgctaaggatgatttctggaattcgcggccgcttctagaa